SEQUENCE LISTING (55 sequences)

SEQ ID NO: 1 - DNA, 26 bp, Tomato mosaic virus
ctcgcaaagtttcgaaccaaatcctc

SEQ ID NO: 2 - DNA, 35 bp, Tomato mosaic virus
cggggtacctgggccccaaccgggggttccggggg

SEQ ID NO: 3 - DNA, 41 bp, Tomato mosaic virus
tcctcgagcctaggctcgcaaagtttcgaaccaaatcctca

SEQ ID NO: 4 - DNA, 35 bp, Tomato mosaic virus; identical to SEQ ID NO: 2

SEQ ID NO: 5 - DNA, 30 bp, Tomato phytoene
tgctcgagtgtgttcttcagttttctgtca

SEQ ID NO: 6 - DNA, 30 bp, Tomato phytoene
aactcgagcgctttgatttctccgaagctt

SEQ ID NO: 7 - DNA, 31 bp, Ribgrass mosaic virus
tactcgaggttcataagaccgcggtaggcgg

SEQ ID NO: 8 - DNA, 36 bp, Ribgrass mosaic virus
cggggtacctgggcccctacccggggtttagggagg

SEQ ID NO: 9 - DNA, 1543 bp, Ribgrass mosaic virus
ctcgaggttcataagaccgcggtaggcggagcgtttgtttactgtagtataattaaatatttgtcagataaaaggttgtttaaagatttgttttttgtttgactgagtcgataatgtcttacgagcctaaagttagtgacttccttgctcttacgaaaaaggaggaaattttacccaaggctttgacgagattaaagactgtctctattagtactaaggatgttatatctgttaaggagtctgagtccctgtgtgatattgatttgttagtgaatgtgccattagataagtataggtatgtgggtgttttgggtgttgttttcaccggtgaatggctggtaccggatttcgttaaaggtggggtaacagtgagcgtgattgacaaacggcttgaaaattccagagagtgcataattggtacgtaccgagctgctgtaaaggacagaaggttccagttcaagctggttccaaattacttcgtatccattgcggatgccaagcgaaaaccgtggcaggttcatgtgcgaattcaaaatctgaagatcgaagctggatggcaacctctagctctagaggtggtttctgttgccatggttactaataacgtggttgttaaaggtttgagggaaaaggtcatcgcagtgaatgatccgaacgtcgaaggtttcgaaggtgtggttgacgatttcgtcgattcggttgctgcattcaaggcgattgacagtttccgaaagaaaaagaaaaagattggaggaagggatgtaaataataataagtatagatatagaccggagagatacgccggtcctgattcgttacaatataaagaagaaaatggtttacaacatcacgagctcgaatcagtaccagtatttcgcagcgatgtgggcagagcccacagcgatgcttaaccagtgcgtgtctgcgttgtcgcaatcgtatcaaactcaggcggcaagagatactgttagacagcagttctctaaccttctgagtgcgattgtgacaccgaaccagcggtttccagaaacaggataccgggtgtatattaattcagcagttctaaaaccgttgtacgagtctctcatgaagtcctttgatactagaaataggatcattgaaactgaagaagagtcgcgtccatcggcttccgaagtatctaatgcaacacaacgtgttgatgatgcgaccgtggccatcaggagtcaaattcagcttttgctgaacgagctctccaacggacatggtctgatgaacagggcagagttcgaggttttattaccttgggctactgcgccagctacataggcgtggtgcacacgatagtgcatagtgtttttctctccacttaaatcgaagagatatacttacggtgtaattccgcaagggtggcgtaaaccaaattacgcaatgttttaggttccatttaaatcgaaacctgttatttcctggatcacctgttaacgtacgcgtggcgtatattacagtgggaataactaaaagtgagaggttcgaatcctccctaaccccgggtaggggccca

SEQ ID NO: 10 - DNA, 54 bp, Ribgrass mosaic virus
gatggcgccttaatacgactcactatagttttatttttgttgcaacaacaacaa

SEQ ID NO: 11 - DNA, 30 bp, Ribgrass mosaic virus
cttgtgcccttcatgacgagctatatcacg

SEQ ID NO: 12 - DNA, 496 bp, Ribgrass mosaic virus
ccttaatacgactcactatagttttatttttgttgcaacaacaacaacaaattacaataacaacaaaacaaatacaaacaacaacaacatggcacaatttcaacaaacagtaaacatgcaaacattgcaggctgccgcagggcgcaacagcctggtgaatgatttagcctcacgacgtgtttatgacaatgctgtcgaggagctaaatgcacgctcgagacgccctaaggttcattactccaaatcagtgtctacggaacagacgctgttagcttcaaacgcttatccggagtttgagatttcctttactcatacccaacatgccgtacactcccttgcgggtggcctaaggactcttgagttagagtatctcatgatgcaagttccgttcggttctctgacgtacgacatcggtggtaactttgcagcgcaccttttcaaaggacgcgactacgttcactgctgtatgccaaacttggatgtacgtgatatagct

SEQ ID NO: 13 - DNA, 55 bp, Ribgrass mosaic virus
gatggcgccttaatacgactcactatagttttatttttgttgcaacaacaacaac

SEQ ID NO: 14 - DNA, 37 bp, Ribgrass mosaic virus
atcgtttaaactgggcccctacccggggttagggagg

SEQ ID NO: 15 - DNA, 497 bp, Ribgrass mosaic virus
ccttaatacgactcactatagttttatttttgttgcaacaacaacaacaaattacaataacaacaaaacaaatacaaacaacaacaacatggcacaatttcaacaaacagtaaacatgcaaacattccaggctgccgcagggcgcaacagcctggtgaatgatttagcctcacgacgtgtttatgacaatgctgtcgaggagctaaatgcacgctcgagacgccctaaggttcattactccaaatcagtgtctacggaacagacgctgttagcttcaaacgcttatccggagtttgagatttcctttactcatacccaaacatgccgtacactcccttgcgggtggcctaaggactcttgagttagagtatctcatgatgcaagttccgttcggttctctgacgtacgacatcggtggtaactttgcagcgcaccttttcaaaggacgcgactacgttcactgctgtatgccaaacttggatgtacgtgatatagct

SEQ ID NO: 16 - DNA, 54 bp, Ribgrass mosaic virus; identical to SEQ ID NO: 10

SEQ ID NO: 17 - DNA, 36 bp, Ribgrass mosaic virus
atcgtttaaactgggcccctacccggggttagggag

SEQ ID NO: 18 - DNA, 14 bp, Arabidopsis thaliana
tcgagcggccgcat

SEQ ID NO: 19 - DNA, 8 bp, Arabidopsis thaliana
gcggccgc

SEQ ID NO: 20 - DNA, 773 bp, Arabidopsis thaliana
ccgaaacattcttcgtagtgaagcaaaatggggttgagtttcgccaagctgtttagcaggctttttgccaagaaggagatgcgaattctgatggttggtcttgatgctgctggtaagaccacaatcttgtacaagctcaagctcggagagattgtcaccaccatccctactattggtttcaatgtggaaactgtggaatacaagaacattagtttcaccgtgtgggatgtcgggggtcaggacaagatccgtcccttgtgagacactacttccagaacactcaaggtctaatctttgttgttgatagcaatgacagagacagagttgttgaggctcgagatgaactccacaggatgctgaatgaggacgagctgcgtgatgctgtgttgcttgtgtttgccaacaagcaagatcttccaaatgctatgaacgctgctgaaatcacagataagcttggccttcactccctccgtcagcgtcattggtatatccagagcacatgtgccacttcaggtgaagggctttatgaaggtctggactggctctccaacaacatcgctggcaaggcatgatgagggagaaattgcgttgcatcgagatgattctgtctgctgtgttgggatctctctctgtcttgatgcaagagagattataaatattatctgaacctttttgcttttttgggtatgtgaatgtttcttattgtgcaagtagatggtcttgtacctaaaaatttactagaagaacccttttaaatagctttcgtgtattgt

SEQ ID NO: 21 - DNA, 404 bp, Arabidopsis thaliana
tccgaaacattcttcgtactgaagcaaaatggggttgagtttcgccaagctgtttagcaggctttttgccaagaaggagatgcgaattctgatggttggtcttgatgctgctggtaagaccacaatcttgtacaagctcaagctcggagagattgtcaccaccatcccttactattggtttcaatgtggaaactgtggaatacaagaacattagtttcaccgtgtggatgtcgggggtcaggacaagatccgtccgtcccttgtggagacactacttccagaacactcaaggtctaatctttgttgttgatagcaatgacagagacagagttgttgaggctcgagatgaactccacaggatgctgaatgaggacgagctgcgtgatgctgtgttgcttgtgttt

SEQ ID NO: 22 - DNA, 400 bp, Arabidopsis thaliana
  misc_feature (1)...(400): n = a, t, c or g
tccgaaacattcttcgtagtgaagcaaaatggggttgagtttcgccaagctgtttagcaggctttttgccaagaaggagatgcgaattctgatggttggtcttgatgctgctggtaagaccacaatgttgtacaagctcaagctcggagagattgtcaccaccatccctactattggtttcaatgtggaaactgtggaatacaagaacattagtttcaccgtgtgggatgtcgggggtcaggacaagatccgtcccttgtggagacactacttccagaacactcaaggtctaatctttgttgttgatagcaatgacagagacagagttgttgaggctcgagatgaactccacaggatgctgnatgagnacgagctgcgtgatgctgtgttgcttgtgttt

SEQ ID NO: 23 - DNA, 550 bp, Arabidopsis thaliana
aaatggggttgagtttcgccaagctgtttagcaggctttttgccaagaaggagatgcgaattctgatggttggtcttgatgctgctggtaagaccacaatcttgtacaagctcaagctcggagagattgtcaccaccatccctactattggtttcaatgtggaaactgtggaatacaagaacattagtttcaccgtgtgggatgtcgggggtcaggacaagatccgtcccttgtggagacactacttccagaacactcaaggtctaatctttgttgttgatagcaatgacagagacagagttgttgaggctcgagatgaactccacaggatgctgaatgaggacgagctgcgtgatgctgtgttgcttgtgtttgccaacaagcaagatcttccaaatgctatgaacgctgctgaaatcacagataagcttggccttcactccctccgtcagcgtcattggtatatccagagcacatgtgccacttcaggtgaagggctttatgaaggtctggactggctctccaacaacatcgctggcaaggcatgatg

SEQ ID NO: 24 - DNA, 550 bp, Oryza sativa
agatggggctcacgttcacgaagctgttcagccgcctcttcgccaagaaggagatgaggatcctcatggtcggtctcgatgcggccggtaaaaccaccatcctctacaagctcaagctcggcgagatcgtcaccactatccccaccatcggttttaatgtcgaaactgttgagtacaagaacattagcttcaccgtttgggatgttggtggtcaggacaagatcaggcccctgtggaggcactatttccagaacacccagggcctcatttttgttgtggacagcaatgacagagagcgtgttgttgaggccagggatgagctccaccgtatgctgaatgaggatgagctacgtgatgctgtgctgctggtgtttgcaaacaaacaagatcttcctaatgccatgaacgctgctgagatcaccgacaagcttggtctgcactccttgcgccagcggcactggtacatccagagcacatgtgctacctctggtgaggggttgtatgaggggcttgactggctttccaacaacattgccaacaaggcttgaag

SEQ ID NO: 25 - DNA, 546 bp, Arabidopsis thaliana
atgggcaatattttcggcaacctgcttaagacccttattggcaagaaggagatgcgaattctgatggttgctcttgatgctgctgctaagaccacaatcttgtacaagctcaagctcggagagattctcaccaccatccctactattggtttcaatgtcgaaactgtggaatacaagaacattactttcaccgtgtgggatgtcgggggtcaggacaagatccgtcccttgttggagacactacttccagaacactcaaggtctaatctttgttgttgatagcaatcacagagagagagttaatgaggctcgagaagaactcatgaggatgctggctgaggacgagctgcgtgatgctgtgttgcttgtgtttgccaacaagcaagatcttccaaatgctatgaacgctgctgaaatcacagataagcttggccttcactccctccctcaccgtaattggtatatccaggccacatgtgccacttcaggtgacgggctttatgaaggtctggactggctcgccaaccagctcaaaacaagaagtga

SEQ ID NO: 26 - DNA, 546 bp, Homo sapiens
  CDS (1)...(546)
atgggcaatatctttggaaaccttctcaagagcctgattgggaacaaggagatgcgcatcctgatggtgggcctggatgccgcaggaaagaccaccatcctatacaagctgaaactgggggagatcgtcaccaccatccctaccattgggttcaatgtggagacagtggagtataagaacatcagctttacagtgtgggatgtgggtggccaggacaagattcgacccctctggagacactacttccagaacacccaagggttgatatttgtggtcgacagcaatgatcggcagcgagtaaatgaggcccgggttgacctgatgagaatgctggcggaggacgagctccgggatgctgtactccttgtctttgcaaacaaacagcatctgcctaatgctatgaacgctgctgagatcacagacaagctgcgcctgcattcccttcgtcaccgtaactggtacattcaggccacctgtcccaccagcggcgacgggctgtacgaaggcctcgactggctggccaatcagctcaaaaacaacaagtga

SEQ ID NO: 27 - protein, 181 aa, Homo sapiens
MGNIFGNLLKSLIGNKEMRILMVGLDAAGKTTILYKLKLGEIVTTIPTIGFNVETVEYKNISFTVWDVGGQDKIRPLWRHYFQNTQGLIFVVDSNDRQRVNEARVDLMRMLAEDELRDAVLLVFANKQHLPNAMNAAEITDKLRLHSLRHRNWYIQATCPTSGDGLYEGLDWLANQLKNNK

SEQ ID NO: 28 - protein, 180 aa, Arabidopsis thaliana
MGLSFAKLFSRLFAKKEMRIMVGLDAAGKTTILYKLKLGEIVTTIPTIGFNVETVEYKNISFTVWDVGGQDKIRPLEWREYFQNTQGLIFVVDSNDRDRVVEARDELERMLNEDELRDAVLLVFANKQDLPNAMNAAEITDKLGLHSLRQRHWYIQSTCATSGEGLYEGLDWLSNNIAGK

SEQ ID NO: 29 - protein, 179 aa, Homo sapiens
MGNIFGNLLKSLIGKKEMRILMVGLDAAGKTTILYKLKLGEIVTTIPTIGFNVETVEKYNISFTVWDVGGQDKIRPLWRHYFQNTQGLIFVVDSNDRERVNEARELMRMLAEDELRDAVLLVFANKQDLPNAMNAAEITDKLGLHSLRHRNWYIQATCATSGDGLYEGLDWLANQLKNK

SEQ ID NO: 30 - DNA, 389 bp, Arabidopsis thaliana
tggtcttgatgctgctggtaagaccacaatcttgtacaagctcaagctcggagagattgtcaccaccatccctactattggtttcaatgtggaaactgtggaatacaagaacattagtttcaccgtgggatgtcgggggtcaggacaagatccgtcccttgtggagacactacttccagaacactcaaggtctaatctttgttgttgatagcaatgacagagacagagttgttgaggctcgagatgaactccacaggatgctgaatgaggacgagctgcgtgatgctgtgttgcttgtgtttgccaacaagcaagatcttccaaatgctatgaacgctgctgaaatcacagataagcttggccttcactccctccgtcagcgtcattgg

SEQ ID NO: 31 - DNA, 391 bp, N. benthamiana
cggtcttgatgcagctggtaaaaccaccatattgtacaagctcaagctgggagagatagttaccactattcctaccattggattcaatgtggagactgttgaatacaagaacataagcttcacggtctgggatgttggtggtcaggacaagatccgaccattgtggaggcattacttccaaaacacacaaggacttatctttgtggtcgatagtaatgatcgtgatcgtgttgttgaggctagagatgagctgcaccggatgttgaatgaggatgaactgagggatgctgtgctgcttgtgtttgctaacaagcaagatcttccaaatgctatgaatgctgctgagattactgacaagcttggtcttcattctctccgtcaacgtcactgg

SEQ ID NO: 32 - DNA, 585 bp, Arabidopsis thaliana
  CDS (1)...(312)
tttcgatctaaggttcgtgatctccttcttctctacgaagtttacactttttcttcaaaggaaacaatgagccagtacaatcaacctcccgttggtgttcctcctcctcaaggttatccaccggagggatatccaaaagatgcttatccaccacaaggatatcctcctcagggatatcctcagcaaggctatccacctcagggatatcctcaacaaggttatcctcagcaaggatatcctccaccgtacgcgcctcaatatcctccaccaccgcaagcatcagcaacaacagagcaagtcctggctttctagaaggatgtcttgctgctctgtgttgttgctgtctcttggatgcttgcttctgattggagtctctctctctctgcataaagcttagggatttatttgtaagagggtttttgggttaaacaaaaaccttaattgatttgtggggcattaaaaatgaatctctcgatgattctcttcgtttatgtggtaatgttcttcggttataacatttaacattgctatcgacgttctgcctagttggatttgattattgggaatgtaaattggttgggaaga

SEQ ID NO: 33 - protein, 103 aa, Arabidopsis thaliana
FRSKVRDLLLLYEVYTFSSKETMSQYNQPPVGVPPPQGYPPEGYPKDAYPPQGYPPQGYPQQGYPPQGYPQQGYPQQGYPPPYAPQYPPPPQASATTEQVLAF

SEQ ID NO: 34 - DNA, 95 bp, Arabidopsis thaliana
aacaatgagccagtacaatcaacctcccgttggtgttcctcctcctcaaggttatccaccggagggatatccaaaagatgcttatccaccacaag

SEQ ID NO: 35 - DNA, 95 bp, Arabidopsis thaliana
aacaatgagccagtacaatcaacctcccgtcggcgttcctcctcctcaaggttatccaccggagggatacccgaaggatgcgtatccaccgcagg

SEQ ID NO: 36 - DNA, 100 bp, Arabidopsis thaliana
tatccaccacaaggatatcctcctcagggatatcctcagcaaggctatccacctcagggatatcctcaacaaggttatcctcagcaaggatatcctccac

SEQ ID NO: 37 - DNA, 100 bp, octopus rhodopsin
tacccaccacaaggctacccaccacaaggctacccacctcaaggctacccaccccagggagcaccaccccaagtagaggcaccccaaggagcaccacccc

SEQ ID NO: 38 - protein, 51 aa, Arabidopsis thaliana
PPVGVPPPQGYPPEGYPKDAYPPQGYPPQGYPQQGYPPQGYPQQGYPQQGY

SEQ ID NO: 39 - protein, 47 aa, octopus rhodopsin
PPQGAYPPPQGYPPQGYPPQGYPPQGAPPQVEAPQGAPPQGVDNQAY

SEQ ID NO: 40 - DNA, 543 bp, Arabidopsis thaliana
cttaaaagcaatatgacagtagagaagatctctcacaaaagacccaaaatcgagtcgtgcaaaattgtacgaacaacaaaatttaaaattcagtccttatcaaagatccaatccagctgcaactagcaacattggcttaacgcttcttagacacaccaacagtctttcctctgcgaccagttgtcttggtgtgttgtccacgaacacggagaccccagtaatgtctcagaccacgatggtttctgattttcttgagacgctcaagatcatccctgagcttcatgtcaagggcattggagacaacttgagagtacttcccatccttgtaatctttctgtctgttcaaaaaccagtctggaatcttgaactgtcttgggtttgcaacaatagtcatgaggttgtcaatctcagctgcagataactcaccagccctcttgttcatgtcgacatcggctttcttgcagacaatgttggccaatctccttccaatacctttgatagaggtaagggcaaacataatcttttgcttaccatcaacggtag

SEQ ID NO: 41 - DNA, 757 bp, Arabidopsis thaliana
ctgacataagttatgttctttgcgaaaataaaagttattccacaaacgcattcgataaaacattcaaaaccttcttcagagtctaatccgtgaactgatgatcgatatagcttcacactatatatcctcttcacttcttagacttcttcttcggtacagctgcagttggagcaggtgtagcagcaggtgctggagcagctacaggcgcaacatctccaccgggacccttagctaaacgctcctctctcctagcatgcttcctttctcggctagccttgttcttcgccctcttagcctcaaactgatcaagacagagtcttctccctagccttctcaagcctttgacttgtggatactctccatcaagacacgcttgttcttgaacacatttacccttaacacgcatgtacatggtcatggtacatgtgcttgtcaatcttctttcgtctctctggatttcttcaaacagacgcctaagaaacacgccttcctacgcattccacagtacctttggttgggaacctaacttacgggtaccccttccttttaaccgattccagagtggcgaccctttatcttggcaatcttcattttgcgagcttggaacaagagtgaatcttgggtggcttctgatgatgaaacctctttaaactttctgaggttttggcggaaatggctgaaacggatttgtgggaccaaccaaattgcctttcggcttaatactgatgcgaccgtttgagtaaaaaccgccttcagg

SEQ ID NO: 42 - DNA, 26 bp, Nicotiana benthamiana
aagaaggagatgcgaattctgatggt

SEQ ID NO: 43 - DNA, 26 bp, Nicotiana benthamiana
atgttgttggagagccagtccagacc

SEQ ID NO: 44 - DNA, 34 bp, Homo sapiens
tacctagggcaatatctttggaaaccttctcaag

SEQ ID NO: 45 - DNA, 38 bp, Homo sapiens
cgctcgagtcacttcttgtttttgagctgattggccag

SEQ ID NO: 46 - DNA, 11 bp, Tobacco mosaic virus
tatagtatttt

SEQ ID NO: 47 - DNA, 11 bp, Tobacco mosaic virus
tataggtattt

SEQ ID NO: 48 - DNA, 11 bp, Tobacco mosaic virus
  misc_feature (1)...(11): n = a, t, c or g
tatagntattt

SEQ ID NO: 49 - DNA, 13 bp, Tobacco mosaic virus
  misc_feature (1)...(13): n=a,t,c or g
tatagtngtattt

SEQ ID NO: 50 - DNA, 12 bp, Nicotiana benthamiana
tataggtatttt

SEQ ID NO: 51 - DNA, 17 bp, Nicotiana benthamiana
gtctatagtcgtatttt

SEQ ID NO: 52 - DNA, 15 bp, Nicotiana benthamiana
  misc_feature (1)...(15): n=a,t,c or g
tatagtngtngtatt

SEQ ID NO: 53 - DNA, 24 bp, Nicotiana benthamiana
  misc_feature (1)...(24): n = a,t,c or g
tatagtngtngtngtngtngtatt

SEQ ID NO: 54 - DNA, 16 bp, Nicotiana benthamiana
tatagtatttgtattt

SEQ ID NO: 55 - DNA, 12 bp, Tobacco mosaic virus
tatagtcgtatt

(19) United States
(12) Patent Application Publication
(10) Pub. No.: US 2003/0077619 A1
(43) Pub. Date: Apr. 24, 2003

(54) Method of isolating human cDNAs by transfecting a nucleic acid sequence of a non-plant donor into a host plant in an anti-sense orientation

(76) Inventors: Monto H. Kumagai, Davis, CA (US); Guy R. della-Cioppa, Vacaville, CA (US); Robert L. Erwin, Vacaville, CA (US); David R. McGee, Vacaville, CA (US)

Correspondence Address: HOWREY SIMON ARNOLD & WHITE, LLP, BOX 34, 301 RAVENSWOOD AVE., MENLO PARK, CA, 94025, US

(21) Appl. No.: 10/142,077

(22) Filed: May 8, 2002

Related U.S. Application Data
(62) Division of application No. 09/359,297, filed on Jul. 21, 1999.
Application No. 09/359,297 is a continuation-in-part of application No. 09/232,170, filed on Jan. 15, 1999, now abandoned.
Application No. 09/232,170 is a continuation-in-part of application No. 09/008,186, filed on Jan. 16, 1998.

(51) Int. Cl. C12Q 1/68; A01H 1/00

(52) U.S. Cl. 435/6; 800/288

(57) ABSTRACT

The present invention provides a method of compiling a functional gene profile of an organism, a method of changing the phenotype or biochemistry of a plant, a method of determining a change in phenotype or biochemistry of a plant, a method of determining the presence of a trait in plant, and a method of isolating human cDNA. The methods comprise expressing transiently a nucleic acid sequence of a non-plant donor organism into a host plant by a viral vector to affect phenotypic or biochemical changes in the host plant. The present invention provides a method for discovering the presence of a new gene and determining its function and sequence in a donor organism such as human by transfecting a nucleic acid sequence of the donor organism into a host plant to knock out the endogenous gene expression.

This application is a divisional application of U.S. patent application Ser. No. 09/359,297, filed Jul. 21, 1999; which is a continuation-in-part application of U.S. patent application Ser. No. 09/232,170, filed on Jan. 15, 1999; which is a continuation-in-part application of U.S. patent application Ser. No. 09/008,186; filed on Jan. 16, 1998. All the above applications are incorporated herein by reference.

### FIELD OF THE INVENTION

The present invention relates generally to the field of molecular biology and plant genetics. Specifically, the present invention relates to a method for determining the presence of a trait in a plant host, and a method of changing the phenotype or biochemistry of a plant host by a transient expression of a nucleic acid sequence from Monera, Protista, Fungi, or Animalia, in an antisense orientation in a host. This invention also relates to a method for identifying a human nucleic acid sequence that silences an endogenous gene of a host plant.

### BACKGROUND OF THE INVENTION

Great interest exists in launching genome projects in plants comparable to the human genome project. Valuable and basic agricultural plants, including corn, soybeans and rice are targets for such projects because the information obtained thereby may prove very beneficial for increasing world food production and improving the quality and value of agricultural products. The United States Congress is considering launching a corn genome project. By helping to unravel the genetics hidden in the corn genome, the project could aid in understanding and combating common diseases of grain crops. It could also provide a big boost for efforts to engineer plants to improve grain yields and resist drought, pests, salt, and other extreme environmental conditions. Such advances are critical for a world population expected to double by 2050. Currently, there are four species which provide 60% of all human food: wheat, rice, corn, and potatoes, and the strategies for increasing the productivity of these plants is dependent on rapid discovery of the presence of a trait in these plants, and the function of unknown gene sequences in these plants. Moreover, such information could identify genes and products encoded by genes useful for human and animal health care such as pharmaceuticals.

One strategy that has been proposed to assist in such efforts is to create a database of expressed sequence tags (ESTs) that can be used to identify expressed genes. Accumulation and analysis of expressed sequence tags (ESTs) have become an important component of genome research. EST data may be used to identify gene products and thereby accelerate gene cloning. Various sequence databases have been established in an effort to store and relate the tremendous amount of sequence information being generated by the ongoing sequencing efforts. Some have suggested sequencing 500,000 ESTs for corn and 100,000 ESTs each for rice, wheat, oats, barley, and sorghum. Efforts at sequencing the genomes of plant species will undoubtedly rely upon these computer databases to share the sequence data as it is generated. Arabidopsis thaliana may be an attractive target discovery of a trait and for gene function discovery because a very large set of ESTs have already been produced in this organism, and these sequences tag more than 50% of the expected Arabidopsis genes.

Potential use of the sequence information so generated is enormous if gene function can be determined. It may become possible to engineer commercial seeds for agricultural use to convey any number of desirable traits to food and fiber crops and thereby increase agricultural production and the world food supply. Research and development of commercial seeds has so far focused primarily on traditional plant breeding, however there has been increased interest in biotechnology as it relates to plant characteristics. Knowledge of the genomes involved and the function of genes contained therein for both monocotyledonous and dicotyledonous plants is essential to realize positive effects from such technology.

The impact of genomic research in seeds is potentially far reaching. For example, gene profiling in cotton can lead to an understanding of the types of genes being expressed primarily in fiber cells. The genes or promoters derived from these genes may be important in genetic engineering of cotton fiber for increased strength or for “built-in” fiber color. In plant breeding, gene profiling coupled to physiological trait analysis can lead to the identification of predictive markers that will be increasingly important in marker assisted breeding programs. Mining the DNA sequence of a particular crop for genes important for yield, quality, health, appearance, color, taste, etc., are applications of obvious importance for crop improvement.

Work has been conducted in the area of developing suitable vectors for expressing foreign DNA and RNA in plant hosts. Ahlquist, U.S. Pat. Nos. 4,885,248 and 5,173,410 describes preliminary work done in devising transfer vectors which might be useful in transferring foreign genetic material into a plant host for the purpose of expression therein. All patent references cited herein are hereby incorporated by reference. Additional aspects of hybrid RNA viruses and RNA transformation vectors are described by Ahlquist et al. in U.S. Pat. Nos. 5,466,788, 5,602,242, 5,627,060 and 5,500,360, all of which are incorporated herein by reference. Donson et al., U.S. Pat. Nos. 5,316,931, 5,589,367 and 5,866,785, incorporated herein by reference, demonstrate for the first time plant viral vectors suitable for the systemic expression of foreign genetic material in plants. Donson et al. describe plant viral vectors having heterologous subgenomic promoters for the systemic expression of foreign genes. Carrington et al., U.S. Pat. No. 5,491,076, describe particular potyvirus vectors also useful for expressing foreign genes in plants. The expression vectors described by Carrington et al. are characterized by utilizing the unique ability of viral polyprotein proteases to cleave heterologous proteins from viral polyproteins. These include Potyviruses such as Tobacco Etch Virus. Additional suitable vectors are described in U.S. Pat. No. 5,811,653 and U.S. patent application Ser. No. 08/324,003, both of which are incorporated herein by reference.

Construction of plant RNA viruses for the introduction and expression of non-viral foreign genes in plants has also been demonstrated by Brisson et al., Methods in Enzymology 118:659 (1986), Guzman et al., Communications in Molecular Biology: Viral Vectors, Cold Spring Harbor Laboratory, pp. 172-189 (1988), Dawson et al., Virology 172:285-292 (1989), Takamatsu et al., EMBO J. 6:307-311 (1987), French et al., Science 231:1294-1297 (1986), and Takamatsu et al., FEBS Letters 269:73-76 (1990). However, these viral vectors have not been shown capable of systemic spread in the plant and expression of the non-viral foreign genes in the majority of plant cells in the whole plant. Moreover, many of these viral vectors have not proven stable for the maintenance of non-viral foreign genes. However, the viral vectors described by Donson et al., in U.S. Pat. Nos. 5,316,931, 5,589,367, and 5,866,785, Turpen in U.S. Pat. No. 5,811,653, Carrington et al. in U.S. Pat. No. 5,491,076, and in co-pending U.S. patent application Ser. No. 08/324,003, have proven capable of infecting plant cells with foreign genetic material and systemically spreading in the plant and expressing the non-viral foreign genes contained therein in plant cells locally or systemically. All patents, patent applications, and references cited in the instant application are hereby incorporated by reference.

With the recent advent of technology for cloning, genes can be selectively turned off. One method is to create antisense RNA or DNA molecules that bind specifically with a targeted gene's RNA message, thereby interrupting the precise molecular mechanism that expresses a gene as a protein. The antisense technology which deactivates specific genes provides a different approach from a classical genetics approach. Classical genetics usually studies the random mutations of all genes in an organism and selects the mutations responsible for specific characteristics. Antisense approach starts with a cloned gene of interest and manipulates it to elicit information about its function.

Post-transcriptional gene silencing (PTGS) in transgenic plants is the manifestation of a mechanism that suppresses RNA accumulation in a sequence-specific manner. There are three models to account for the mechanism of PTGS: direct transcription of an antisense RNA from the transgene, an antisense RNA produced in response to over expression of the transgene, or an antisense RNA produced in response to the production of an aberrant sense RNA product of the transgene (Baulcombe, Plant Mol. Biol. 32:79-88 (1996)). The PTGS mechanism is typified by the highly specific degradation of both the transgene mRNA and the target RNA, which contains either the same or complementary nucleotide sequences (Waterhouse et al Proc. Natl. Acad. Sci. USA 10: 13959-64 (1998)). Antisense RNA has been used to down regulate gene expression in transgenic and transfected plants. The effectiveness of antisense on the inhibition of eukaryotic gene expression was first demonstrated by Izant et al. (Cell 36(4): 1007-1015 (1984)). Since then, the down-regulation of different genes from transgenic plants has been reported. Kumagai et al (Proc. Natl. Acad. Sci. USA 92:1679 (1995)) report that gene expression in transfected Nicotiana benthamiana was cytoplasmic inhibited by viral delivery of a RNA of a known sequence derived from cDNA encoding tomato phytoene desaturase in a positive sense or an antisense orientation. The host plant, Nicotiana benthamiana, and the donor plant, tomato (Lycopersicon esculentum), belong to the same family. There is also evidence that inhibition of endogenous genes occurs in transgenic plants containing sense RNA (Van der Krol et al., Plant Cell 2(4):291-299 (1990), Napoli et al., Plant Cell 2:279-289 (1990) and Fray et al., Plant Mol. Biol. 22:589-602 (1993)).

The antisense technology can be used to develop a functional genomic screening of a donor organism such as Monera, Protisca, Fungi, or Animalia. The antisense technology is applied in this invention to provide a method of discovering the presence of a trait in a plant, a method of determining the function and sequence of a nucleic acid of a donor organism, and a method of isolating a cDNA of a donor organism by expressing the nucleic acid sequence that has not been identified in a host plant. GTP-binding proteins exemplify this invention. In eukaryotic cells, GTP-binding proteins function in a variety of cellular processes, including signal transduction, cytoskeletal organization, and protein transport. The heterotrimeric and monomeric GTP-binding proteins that may be involved in secretion and intracellular transport are divided into two structural classes: the rab and the ARF families. The ARFs are highly conserved and found in all eukaryotic cells including human, yeast, plants, and slime mold. The cDNAs encoding GTP binding proteins have been isolated from a variety of plants including rice, barley, corn, tobacco, and A. thaliana. For example, Verwoert et al. (Plant Molecular Biol. 27:629-633 (1995)) report the isolation of a Zea mays cDNA clone encoding a GTP-binding protein of the ARF family by direct genetic selection in an E. coli fabD mutant with a maize cDNA expression library. Regad et al. (FEBS 2:133-136 (1993)) isolated a cDNA clone encoding the ARF from a cDNA library of Arabidopsis thaliana cultured cells by randomly selecting and sequencing cDNA clones. Dallmann et al. (Plant Molecular Biol. 19:847-857 (1992)) isolated two cDNAs encoding small GTP-binding proteins from leaf cDNA libraries using a PCR approach. Dallmann et al. prepared leaf cDNAs and use them as templates in PCR amplifications with degenerated oligonucleotides corresponding to the highly conserved motifs, found in members of the ras superfamily, as primers. Haizel et al., (Plant J., 11:93-103 (1997)) isolated cDNA and genomic clones encoding Ran-like small GTP binding proteins from Arabidopsis cDNA and genomic libraries using a full-length tobacco Nt Ran1 cDNA as a probe. The present invention provides advantages over the above isolation methods in that it only sequences clones that have a function and does not randomly sequence clones. The nucleic acid inserts in clones that have a function are labeled and used as probes to isolate cDNAs that hybridize to them.

For the production of some products, including products for the human health industry, plants provide an optimal system because of reduced capital costs and the greater potential for large-scale production compared with microbial or animal systems. Foreign genes can be expressed in plants either by permanent insertion into the genome or by transient expression using virus-based vectors. Each approach has its own distinct advantages. Transformation for permanent expression needs to be done only once, whereas each generation of plants needs to be inoculated with the transient expression vector. However, virus-based expression systems, in which the foreign mRNA is greatly amplified by virus replication, can produce very high levels of certain proteins in leaves and other tissues. Similar levels of foreign protein production in transgenic plants often are unattainable, in some cases because of gene silencing. Viral vector-produced protein can be directed to specific subcellular locations, such as endomembrane, cytosol, or organelles, or it can be attached to macromolecules, such as virions, which aids purification of the protein.

The present invention provides a method for discovering the presence of a trait in a plant by expressing a nucleic acid sequence of a donor organism in an antisense orientation in a host plant. Once the presence of a trait is identified by phenotypic changes, the nucleic acid insert in the cDNA clone or in the vector is then sequenced. The present method provides a rapid method for determining the presence of a trait in a host plant and a method for identifying a nucleic acid sequence and its function of a donor organism by screening a host plant transfected by the nucleic acid sequence of the donor organism for phenotypic or biochemical change in the host plant.

### SUMMARY OF THE INVENTION

The present invention is directed to a method of changing the phenotype or biochemistry of a host organism, a method of determining a change in phenotype or biochemistry in a host organism, and a method of determining the presence of a trait in a host organism. The method comprises the steps of expressing transiently a nucleic acid sequence of a donor organism in an antisense orientation in a host organism, identifying changes in the host organism, and correlating the expression with the phenotypic changes. The nucleic acid sequence does not need to be isolated, identified, or characterized prior to transfection into the host plant. The donor organism and the host plant belong to different kingdoms. The present invention is also directed to a method of making a functional gene profile in a host organism by transiently expressing a nucleic acid sequence library in a host plant, determining the phenotypic or biochemical changes in the host organism, identifying a trait associated with the change, identifying the donor gene associated with the trait, and identifying the homologous host gene, if any. The present invention is also directed to a method of determining the function of a nucleic acid sequence, including a gene, in a donor organism, by transfecting the nucleic acid sequence into a host plant in a manner so as to affect phenotypic changes in the host plant. In one embodiment, recombinant viral nucleic acids are prepared to include the nucleic acid insert of a donor. The recombinant viral nucleic acids infect a host plant and produce antisense RNAs in the cytoplasm which result in a reduced expression of endogenous cellular genes in the host plant. Once the presence of a trait is identified by phenotypic changes, the function of the nucleic acid is determined. The nucleic acid insert in a cDNA clone or in a vector is then sequenced. The nucleic acid sequence is determined by a standard sequence analysis.

The present invention is also directed to a method of increasing yield of a grain crop. The method comprises expressing transiently a nucleic acid sequence of a donor organism in an antisense orientation in a grain crop, for example, in the cytoplasm of the grain crop, wherein said expressing results in stunted growth and increased seed production of the grain crop. A preferred method comprises the steps of cloning the nucleic acid sequence into a plant viral vector and infecting the grain crop with a recombinant viral nucleic acid comprising said nucleic acid sequence.

One aspect of the invention is a method of identifying and determining a nucleic acid sequence in a donor organism, whose function is to silence endogenous genes in a host plant, by introducing the nucleic acid into the host plant by way of a viral nucleic acid such as a plant viral nucleic acid suitable to produce expression of the nucleic acid in the transfected host. This method utilizes the principle of post-transcription gene silencing of the endogenous host gene homolog, for example, antisense RNAs. Particularly, this silencing function is useful for silencing a multigene family frequently found in plants.

Another aspect of the invention is to discover genes in a non-plant organism having the same function as that in a plant. The method starts with building a cDNA library, or a genomic DNA library, or a pool of RNA of a non-plant organism, for example, a human. Then, a recombinant viral nucleic acid comprising a nucleic acid insert derived from the library is prepared and is used to infect a host plant. The infected host plant is inspected for phenotypic changes. The recombinant viral nucleic acid that results in phenotypic changes in the host plant is identified and the sequence of the nucleic acid insert is determined by a standard method. Such nucleic acid sequence in the donor organism has substantial sequence homology as that in the host plant: the nucleic acid sequences are conserved between the non-plant organism and the plant. Once the nucleic acid is sequenced, it can be labeled and used as a probe to isolate full-length cDNAs from the donor organism or the host plant. After the amino acid sequences derived from the cDNAs of the donor organism and the plant are compared, the plant cDNA sequence can be changed so that it encodes the same amino acid sequence as the cDNA of the donor organism encodes. This invention provides a rapid means for elucidating the function and sequence of nucleic acids of a donor organism; such rapidly expanding information can be subsequently utilized in the field of genomics.

In one embodiment, a nucleic acid is introduced into a plant host wherein the plant host may be a monocotyledonous or dicotyledonous plant, plant tissue or plant cell. Preferably, the nucleic acid is introduced by way of a recombinant plant viral nucleic acid. Preferred recombinant plant viral nucleic acids useful in the present invention comprise a native plant viral subgenomic promoter, a plant viral coat protein coding sequence, and at least one non-native nucleic acid sequence. Some viral vectors used in accordance with the present invention may be encapsidated by the coat proteins encoded by the recombinant plant virus. Recombinant plant viral nucleic acids or recombinant plant viruses are used to infect a plant host. The recombinant plant viral nucleic acid is capable of replication in the host, localized or systemic spread in the host, and transcription or expression of the non-native nucleic acid in the host to produce a phenotypic or biochemical change. Any suitable vector constructs useful to produce localized or systemic expression of nucleic acids in host plants are within the scope of the present invention.

### BRIEF DESCRIPTION OF THE FIGURES

FIG. 1 depicts the Vector TT01 PDS-.

FIG. 2 depicts plasmid pBS712.

FIG. 3 depicts the plasmid KS+TVCVK #23.

FIG. 4 depicts the plasmid pBS 735.

FIG. 5 depicts the plasmid pBS 740.

FIG. 6 depicts the plasmid pBS 740 AT #120. (ATCC No: PTA-325, deposited Jul. 12, 1999, American Type Culture Collection, 10801 University Blvd., Manassas, Va. 20110).

FIG. 7 shows the nucleotide sequence comparison of A. thaliana 740 AT #120 (SEQ ID NO: 21) and A. thaliana est AA042085 (SEQ ID NO: 22).

FIG. 8 shows the nucleotide sequence comparison of 740 AT #120 (SEQ ID NO; 23) and rice Oryza sativa D17760 (SEQ ID NO: 24).

FIG. 9 shows the nucleotide sequence alignment of 740 AT #120H (SEQ ID NO: 25) to human ADP-ribosylation factor (ARF3) M33384 (SEQ ID NO: 26) the amino acid sequence shown is the amino acid sequence of M33384 (SEQ ID NO: 27).

FIG. 10 shows the amino acid sequence alignment of 740 AT #120 (SEQ ID NO: 28) to human ADP-ribosylation factor (ARF3) P16587 (SEQ ID NO: 29).

FIG. 11 shows the KS+Nb ARF #3 (ATCC No: PTA-324, deposited Jul. 12, 1999, American Type Culture Collection, 10801 University Blvd., Manassas, Va. 20110) plasmid map.

FIG. 12 shows the nucleotide sequence comparison of A. thaliana 740 AT #120 (SEQ ID NO: 30) and N. benthamniana KS+Nb ARF#3 (SEQ ID NO: 31).

FIG. 13 depicts the plasmid pBS 740 AT #88 (ATCC No: PTA-331, deposited Jul. 12, 1999, American Type Culture Collection, 10801 University Blvd., Manassas, Va. 20110).

FIG. 14 shows partial nucleotide and amino acid sequences of 740 AT #88 (SEQ ID NOs: 32 and 33).

FIG. 15 shows the nucleotide alignment of 740 AT #88 to Brassica rapa cDNA L35812 (SEQ ID NOs. 34 and 35).

FIG. 16 shows the nucleotide alignment of 740 AT #88 to octopus rhodopsin cDNA X07797 (SEQ ID NOs. 36 and 37).

FIG. 17 shows the amino acid comparison of 740 AT #88 to octopus rhodopsin P31356 (SEQ ID NOs. 38 and 39).

FIG. 18 depicts the plasmid 740 AT #377 (ATCC No: PTA-334, deposited Jul. 12, 1999, American Type Culture Collection, 10801 University Blvd., Manassas, Va. 20110).

FIG. 19 shows the nucleotide sequence of 740 AT #377 (SEQ ID NO: 40).

FIG. 20 depicts the plasmid 740 #2483 (ATCC No: PTA-329, deposited Jul. 12, 1999, American Type Culture Collection, 10801 University Blvd., Manassas, Va. 20110).

FIG. 21 shows the nucleotide sequence of 740 #2483 (SEQ ID NO: 41.).

### DETAILED DESCRIPTION OF THE INVENTION

The present invention is directed to a method of changing the phenotype or biochemistry of a donor organism, a method of determining a change in phenotype or biochemistry in an organism, a method of determining the presence of a trait in an organism, and a method of determining the function and sequence of a nucleic acid in a non-plant organism. The methods comprise the steps of a transient expression of a nucleic acid sequence of a donor organism in an antisense orientation in a host organism; identifying changes in the host organism; and correlating the expression and the changes. The presence of a trait is identified either in the infected host organism or in an uninfected host organism. A preferred host organism includes a plant, a plant tissue or a plant cell. In one preferred embodiment, the method comprising the steps of (a) preparing a library of cDNA, genomic DNA, or a mRNA pool of a donor organism, (b) constructing recombinant viral nucleic acids comprising a nucleic acid insert derived from said library, (c) infecting each host plant with one of recombinant viral nucleic acids, (d) growing infected host plant, and (e) determining changes in the host plant.

The invention is directed to a method of compiling an antisense functional gene profile of an organism. The method comprises (a) preparing a vector library of DNA or RNA sequences from a donor organism, each sequence being in an antisense orientation; (b) infecting a plant host with a vector; (c) transiently expressing the donor DNA or RNA sequence in the growing plant host; (d) determining one or more phenotypic or biochemical changes in the plant host, if any; (e) identifying an associated trait where a phenotypic or biochemical change occurs; (f) identifying a donor gene associated with the trait; (g) identifying a plant host gene, if any, associated with the trait; and (h) repeating steps (b)-(g) until an antisense functional gene profile of the plant host and/or of the donor organism is compiled.

The invention is also directed to a method of compiling a functional gene profile of an organism. The method comprises (a) preparing a vector library of DNA or RNA sequences from a donor organism, each sequence being in either an antisense or a positive orientation; (b) infecting a plant host with a vector; (c) transiently expressing the donor DNA or RNA sequence in the growing plant host; (d) determining one or more phenotypic or biochemical changes in the plant host, if any; (e) identifying an associated trait where a phenotypic or biochemical change occurs; (f) identifying a donor gene associated with the trait; (g) identifying a plant host gene, if any, associated with the trait; and (h) repeating steps (b)-(g) until a functional gene profile of the plant host and/or of the donor organism is compiled. A detailed discussion of positive sense expression of nucleic acids is presented in a co-pending and co-owned U.S. patent application Ser. No. 09/359,300 (Kumagai et al., Attorney Docket No. 08010137US07, filed Jul. 21, 1999), the entire disclosure of which is enclosed herein by reference.

The present method has the advantages that the nucleic acid sequence does not need to be identified, known, or characterized prior to infecting a host plant with a recombinant viral nucleic acid comprising the nucleic acid sequence. Once changes in the host plant is observed, the nucleic acid sequence can be determined by further identifying the recombinant viral nucleic acid that results in changes in the host, and analyzing the sequence of the nucleic acid insert in the recombinant viral nucleic acid that results in changes in the host.

The present invention provides a method of infecting a host plant by a recombinant viral nucleic acid which contains one or more non-native nucleic acid sequences, or by a recombinant virus containing a recombinant viral nucleic acid. The non-native nucleic acids are subsequently transcribed or expressed in the infected host plant. The products of the non-native nucleic acid sequences result in changing phenotypic traits in the host plant, affecting biochemical pathways within the host plant, or affecting endogenous gene expression within the host plant.

In one embodiment, a nucleic acid is introduced into a plant host by way of a recombinant viral nucleic acid. Such recombinant viral nucleic acids are stable for the maintenance and transcription or expression of non-native nucleic acid sequences and are capable of systemically transcribing or expressing such non-native sequences in the plant host. Preferred recombinant plant viral nucleic acids useful in the present invention comprise a native plant viral subgenomic promoter, a plant viral coat protein coding sequence, and at least one non-native nucleic acid sequence.

In a second embodiment, plant viral nucleic acid sequences are characterized by the deletion of a native coat protein coding sequence. The plant viral nucleic acid sequence comprises a non-native plant viral coat protein coding sequence and a non-native promoter, preferably the subgenomic promoter of the non-native coat protein coding sequence. Such plant viral nucleic acid sequence is capable of expressing in a plant host, packaging the recombinant plant viral nucleic acid, and ensuring a systemic infection of the host by the recombinant plant viral nucleic acid. The recombinant plant viral nucleic acid may contain one or more additional native or non-native subgenomic promoters. Each non-native subgenomic promoter is capable of transcribing or expressing adjacent genes or nucleic acid sequences in the plant host and incapable of recombination with each other and with native subgenomic promoters. One or more non-native nucleic acids may be inserted adjacent to the native plant viral subgenomic promoter or the native and non-native plant viral subgenomic promoters if more than one nucleic acid sequence is included. Moreover, two or more heterologous non-native subgenomic promoters may be used. The non-native nucleic acid sequences may be transcribed or expressed in the host plant under the control of the subgenomic promoter to produce the products of the nucleic acids of interest.

In a third embodiment, plant recombinant viral nucleic acids comprise a native coat protein coding sequence instead of a non-native coat protein coding sequence, placed adjacent one of the non-native coat protein subgenomic promoters.

In a fourth embodiment, plant recombinant viral nucleic acids comprise a native coat protein gene adjacent its native subgenomic promoter, one or more non-native subgenomic promoters, and at least one non-native nucleic acid sequence. The native plant viral subgenomic promoter initiates transcription of the plant viral coat protein sequence. The non-native subgenomic promoters are capable of transcribing or expressing adjacent genes in a plant host and are incapable of recombination with each other and with native subgenomic promoters. Non-native nucleic acid sequences may be inserted adjacent the non-native subgenomic plant viral promoters such that the sequences are transcribed or expressed in the host plant under control of the subgenomic promoters to produce a product of the non-native nucleic acid. Alternatively, the native coat protein coding sequence may be replaced by a non-native coat protein coding sequence.

The viral vectors used in accordance with the present invention may be encapsidated by the coat proteins encoded by the recombinant plant virus. The recombinant plant viral nucleic acid or recombinant plant virus is used to infect a host plant. The recombinant plant viral nucleic acid is capable of replication in the host, localized or systemic spread in the host, and transcription or expression of the non-native nucleic acid in the host to affect a phenotypic or biochemical change in the host.

In one embodiment, recombinant plant viruses are used which encode for the expression of a fusion between a plant viral coat protein and the amino acid product of the nucleic acid of interest. Such a recombinant plant virus provides for high level expression of a nucleic acid of interest. The location or locations where the viral coat protein is joined to the amino acid product of the nucleic acid of interest may be referred to as the fusion joint. A given product of such a construct may have one or more fusion joints. The fusion joint may be located at the carboxyl terminus of the viral coat protein or the fusion joint may be located at the amino terminus of the coat protein portion of the construct. In instances where the nucleic acid of interest is located internal with respect to the 5′ and 3′ residues of the nucleic acid sequence encoding for the viral coat protein, there are two fusion joints. That is, the nucleic acid of interest may be located 5′, 3′, upstream, downstream or within the coat protein. In some embodiments of such recombinant plant viruses, a “leaky” start or stop codon may occur at a fusion joint which sometimes does not result in translational termination. A more detailed description of some recombinant plant viruses according to this embodiment of the invention may be found in co-pending U.S. patent application Ser. No. 08/324,003 the disclosure of which is incorporated herein by reference.

The present invention is not intended to be limited to any particular viral constructs, but rather to include all operable constructs. Specifically, those skilled in the art may choose to transfer DNA or RNA of any size up to and including an entire genome in a donor organism into a host organism in order to determine the presence of a trait in the plant. Those skilled in the art will understand that the recited embodiments are representative only. All operable constructs useful to produce localized or systemic expression of nucleic acids in a plant are within the scope of the present invention.

The chimeric genes and vectors and recombinant plant viral nucleic acids used in this invention are constructed using techniques well known in the art. Suitable techniques have been described in Sambrook et al. (2nd ed.), Cold Spring Harbor Laboratory, Cold Spring Harbor (1982, 1989); Methods in Enzymol. (Vols. 68, 100, 101, 118, and 152-155) (1979, 1983, 1986 and 1987); and DNA Cloning, D. M. Clover, Ed., IRL Press, Oxford (1985). Medium compositions have been described by Miller, J., Experiments in Molecular Genetics, Cold Spring Harbor Laboratory, New York (1972), as well as the references previously identified, all of which are incorporated herein by reference. DNA manipulations and enzyme treatments are carried out in accordance with manufacturers' recommended procedures in making such constructs.

The first step in producing recombinant plant viral nucleic acids is to modify the nucleotide sequences of the plant viral nucleotide sequence by known conventional techniques such that one or more non-native subgenomic promoters are inserted into the plant viral nucleic acid without destroying the biological function of the plant viral nucleic acid. The subgenomic promoters are capable of transcribing or expressing adjacent nucleic acid sequences in a plant host infected by the recombination plant viral nucleic acid or recombinant plant virus. The native coat protein coding sequence may be deleted in some embodiments, placed under the control of a non-native subgenomic promoter in other embodiments, or retained in a further embodiment. If it is deleted or otherwise inactivated, a non-native coat protein gene is inserted under control of one of the non-native subgenomic promoters, or optionally under control of the native coat protein gene subgenomic promoter. The non-native coat protein is capable of encapsidating the recombinant plant viral nucleic acid to produce a recombinant plant virus. Thus, the recombinant plant viral nucleic acid contains a coat protein coding sequence, which may be native or a nonnative coat protein coding sequence, under control of one of the native or non-native subgenomic promoters. The coat protein is involved in the systemic infection of the plant host.

Viruses suitable for use according to the methods of the present invention include viruses from the tobamovirus group such as Tobacco Mosaic virus (TMV), Ribgrass Mosaic Virus (RGM), Cowpea Mosaic virus (CMV), Alfalfa Mosaic virus (AMV), Cucumber Green Mottle Mosaic virus watermelon strain (CGMMV-W) and Oat Mosaic virus (OMV) and viruses from the brome mosaic virus group such as Brome Mosaic virus (BMV), broad bean mottle virus and cowpea chlorotic mottle virus. Additional suitable viruses include Rice Necrosis virus (RNV), and geminiviruses such as Tomato Golden Mosaic virus (TGMV), Cassava Latent virus (CLV) and Maize Streak virus (MSV). Each of these groups of suitable viruses is characterized below. However, the invention should not be construed as limited to using these particular viruses, but rather the present invention is contemplated to include all plant viruses at a minimum.

### Tobamovirus Group

The tobacco mosaic virus (TMV) is of particular interest to the instant invention because of its ability to express genes at high levels in plants. TMV is a member of the tobamovirus group. The TMV virion is a tubular filament, and comprises coat protein sub-units arranged in a single right-handed helix with the single-stranded RNA intercalated between the turns of the helix. TMV infects tobacco as well as other plants. TMV virions are 300 nm×18 nm tubes with a 4 nm-diameter hollow canal, and consist of 2140 units of a single structural protein helically wound around a single RNA molecule. The genome is a 6395 base plus-sense RNA. The 5′-end is capped and the 3′-end contains a series of pseudoknots and a tRNA-like structure that will specifically accept histidine. The genomic RNA functions as mRNA for the production of proteins involved in viral replication: a 126-kDa protein that initiates 68 nucleotides from the 5′-terminus, and a 183-kDa protein synthesized by readthrough of an amber termination codon approximately 10% of the time. Only the 183-kDa and 126-kDa viral proteins are required for the TMV replication in trans. (Ogawa et al., Virology 185:580-584 (1991)). Additional proteins are translated from subgenomic size mRNA produced during replication (Dawson, Adv. Virus Res., 38:307-342 (1990)). The 30-kDa protein is required for cell-to-cell movement; the 17.5-kDa capsid protein is the single viral structural protein. The function of the predicted 54-kDa protein is unknown.

TMV assembly apparently occurs in plant cell cytoplasm, although it has been suggested that some TMV assembly may occur in chloroplasts since transcripts of ctDNA have been detected in purified TMV virions. Initiation of TMV assembly occurs by interaction between ring-shaped aggregates (“discs”) of coat protein (each disc consisting of two layers of 17 subunits) and a unique internal nucleation site in the RNA; a hairpin region about 900 nucleotides from the 3′-end in the common strain of TMV. Any RNA, including subgenomic RNAs containing this site, may be packaged into virions. The discs apparently assume a helical form on interaction with the RNA, and assembly (elongation) then proceeds in both directions (but much more rapidly in the 3′ - to 5′- direction from the nucleation site).

Another member of the Tobamoviruses, the Cucumber Green Mottle Mosaic virus watermelon strain (CGMMV-W) is related to the cucumber virus. Nozu et al., Virology 45:577 (1971). The coat protein of CGMMV-W interacts with RNA of both TMV and CGMMV to assemble viral particles in vitro. Kurisu et al., Virology 70:214 (1976).

Several strains of the tobamovirus group are divided into two subgroups, on the basis of the location of the assembly of origin. Subgroup I, which includes the vulgare, OM, and tomato strain, has an origin of assembly about 800-1000 nucleotides from the 3′-end of the RNA genome, and outside the coat protein cistron. Lebeurier et al., Proc. Natl. Acad. Sci. USA 74:149 (1977); and Fukuda et al., Virology 101:493 (1980). Subgroup II, which includes CGMMV-W and cornpea strain (Cc) has an origin of assembly about 300-500 nucleotides from the 3′-end of the RNA genome and within the coat-protein cistron. The coat protein cistron of CGMMV-W is located at nucleotides 176-661 from the 3′-end. The 3′ noncoding region is 175 nucleotides long. The origin of assembly is positioned within the coat protein cistron. Meshi et al., Virology 127:54 (1983).

### Brome Mosaic Virus Group

Brome Mosaic virus (BMV) is a member of a group of tripartite, single-stranded, RNA-containing plant viruses commonly referred to as the bromoviruses. Each member of the bromoviruses infects a narrow range of plants. Mechanical transmission of bromoviruses occurs readily, and some members are transmitted by beetles. In addition to BV, other bromoviruses include broad bean mottle virus and cowpea chlorotic mottle virus.

Typically, a bromovirus virion is icosahedral, with a diameter of about 26 μm, containing a single species of coat protein. The bromovirus genome has three molecules of linear, positive-sense, single-stranded RNA, and the coat protein mRNA is also encapsidated. The RNAs each have a capped 5′-end, and a tRNA-like structure (which accepts tyrosine) at the 3′-end. Virus assembly occurs in the cytoplasm. The complete nucleotide sequence of BMV has been identified and characterized as described by Ahlquist et al., J. Mol. Biol. 153:23 (1981).

### Rice Necrosis Virus

Rice Necrosis virus is a member of the Potato Virus Y Group or Potyviruses. The Rice Necrosis virion is a flexuous filament comprising one type of coat protein (molecular weight about 32,000 to about 36,000) and one molecule of linear positive-sense single-stranded RNA. The Rice Necrosis virus is transmitted by Polymyxa oraminis (a eukaryotic intracellular parasite found in plants, algae and fungi).

### Geminiviruses

Geminiviruses are a group of small, single-stranded DNA-containing plant viruses with virions of unique morphology. Each virion consists of a pair of isometric particles (incomplete icosahedral), composed of a single type of protein (with a molecular weight of about 2.7-3.4×10⁴). Each geminivirus virion contains one molecule of circular, positive-sense, single-stranded DNA. In some geminiviruses (i.e., Cassava latent virus and bean golden mosaic virus) the genome appears to be bipartite, containing two single-stranded DNA molecules.

### Potyviruses

Potyviruses are a group of plant viruses which produce polyprotein. A particularly preferred potyvirus is tobacco etch virus (TEV). TEV is a well characterized potyvirus and contains a positive-strand RNA genome of 9.5 kilobases encoding for a single, large polyprotein that is processed by three virus-specific proteinases. The nuclear inclusion protein “a” proteinase is involved in the maturation of several replication-associated proteins and capsid protein. The helper component-proteinase (HC-Pro) and 35-kDa proteinase both catalyze cleavage only at their respective C-termini. The proteolytic domain in each of these proteins is located near the C-terminus. The 35-kDa proteinase and HC-Pro derive from the N-terminal region of the TEV polyprotein.

The nucleic acid of any suitable virus can be utilized to prepare a recombinant viral nucleic acid for use in the present invention, and the foregoing are only exemplary of such suitable viruses. The nucleotide sequence of the virus can be modified, using conventional techniques, by insertion of one or more subgenomic promoters into the viral nucleic acid. The subgenomic promoters are capable of functioning in a specific host organism. For example, if the host is a tobacco plant, TMV, TEV, or other viruses containing suitable subgenomic promoter may be utilized. The inserted subgenomic promoters should be compatible with the viral nucleic acid and capable of directing transcription or expression of adjacent nucleic acid sequences in tobacco.

The native or non-native coat protein gene is included in the recombinant plant viral nucleic acid. When non-native nucleic acid is utilized, it may be positioned adjacent its natural subgenomic promoter or adjacent one of the other available subgenomic promoters. The non-native coat protein, as is the case for the native coat protein, is capable of encapsidating the recombinant plant viral nucleic acid and providing for systemic spread of the recombinant plant viral nucleic acid in a host plant. The coat protein is selected to provide a systemic infection in the plant host of interest. For example, the TMV-O coat protein provides systemic infection in N. benthamiana, whereas TMV-U1 coat protein provides systemic infection in N. tabacum.

The recombinant viral nucleic acid is prepared by cloning a viral nucleic acid. If the viral nucleic acid is DNA, it can be cloned directly into a suitable vector using conventional techniques. One technique is to attach an origin of replication to the viral DNA which is compatible with the cell to be transfected. If the viral nucleic acid is RNA, a full-length DNA copy of the viral genome is first prepared by well-known procedures. For example, the viral RNA is transcribed into DNA using reverse transcriptase to produce subgenomic DNA pieces, and a double-stranded DNA made using DNA polymerases. The cDNA is then cloned into appropriate vectors and cloned into a cell to be transfected. Alternatively, the cDNA is ligated into the vector and is directly transcribed into infectious RNA in vitro, the infectious RNA is then inoculated onto the host. The cDNA pieces are mapped and combined in a proper sequence to produce a full-length DNA copy of the viral RNA genome, if necessary. DNA sequences for the subgenomic promoters, with or without a coat protein gene, are then inserted into the nucleic acid at non-essential sites, according to the particular embodiment of the invention utilized. Non-essential sites are those that do not affect the biological properties of the viral nucleic acids. Since the RNA genome is the infective agent, the cDNA is positioned adjacent a suitable promoter so that the RNA is produced in the production cell. The RNA can be capped by the addition of a nucleotide using conventional techniques (Dawson et al., Proc. Natl. Acad. Sci. USA, 83:1832 (1986)), between the transcription start site of the promoter and the start of the cDNA of a viral nucleic acid. One or more nucleotides may be added. In a preferred embodiment of the present invention, the inserted nucleotide sequence contains a G at the 5′-end. In one embodiment, the inserted nucleotide sequence is GNN, GTN, or their multiples, (GNN)ₓ or (GTN)ₓ. The capped RNA can be packaged in vitro with added coat protein from TMV to make assembled virions. These assembled virions can then be used to inoculate plants or plant tissues.

Alternatively, an uncapped RNA may be-employed in the embodiments of the present invention. Contrary to the practiced art in scientific literature and in an issued patent (Ahlquist et al., U.S. Pat. No. 5,466,788), uncapped transcripts for virus expression vectors are infective on both plants and in plant cells. Capping is not a prerequisite for establishing an infection of a virus expression vector in plants, although capping increases the efficiency of infection.

One feature of the recombinant plant viral nucleic acids useful in the present invention is that they further comprise one or more non-native nucleic acid sequences capable of being transcribed in a host plant. These nucleic acid sequences may be native nucleic acid sequences that occur in a host plant. Preferably, these nucleic acid sequences are non-native nucleic acid sequences that do not nornmally occur in a host plant. These nucleic acid sequences are derived from a donor organism, which preferably belongs to a non-plant kingdom. Non-plant kingdoms include kingdom Monera, Kingdom Protista, Kingdom Fungi and Kingdom Animalia. Kingdom Monera includes subkingdom Archaebacteriobionta (archaebacteria): division Archaebacteriophyta (methane, salt and sulfolobus bacteria); subkingdom Eubacteriobionta (true bacteria): division Eubacteriophyta; subkingdom Viroids; and subkingdom Viruses. Kingdom Protista includes subkingdom Phycobionta: division Xanthophyta 275 (yellow-green algae), division Chrysophyta 400 (golden-brown algae), division Dinophyta (Pyrrhophyta) 1,000 (dinoflagellates), division Bacillariophyta 5,500 (diatoms), division Cryptophyta 74 (cryptophytes), division Haptophyta 250 (haptonema organisms), division Euglenophyta 550 (euglenoids), division Chlorophyta, class Chlorophyceae 10,000 (green algae), class Charophyceae 200 (stoneworts), division Phaeophyta 900 (brown algae), and division Rhodophyta 2,500 (red algae); subkingdom Mastigobionta 960: division Chytridiomycota 750 (chytrids), and division Oomycota (water molds) 475; subkingdom Myxobionta 320: division Acrasiomycota (cellular slime molds) 21, and division Myxomycota 500 (true slime molds). Kingdom Fungi includes division Zygomycota 570 (coenocytic fungi): subdivision Zygomycotina; and division Eumycota 350 (septate fungi): subdivision Ascomycotina 56,000 (cup fingi), subdivision Basidiomycotina 25,000 (club fungi), subdivision Deuteromycotina 22,000 (imperfect fungi), and subdivision Lichenes 13,500. A preferred donor organism is human. Host plants are those capable of being infected by an infectious RNA or a virus containing a recombinant viral nucleic acid. Host plants include plants of commercial interest, such as food crops, seed crops, oil crops, ornamental crops and forestry crops. For example, wheat, rice, corn, potatoes, barley, tobaccos, soybean canola, maize, oilseed rape, Arabidopsis, and Nicotiana, can be selected as a host plant. Preferred host plants include Nicotiana, preferably, Nicotiana benthamiana, or Nicotiana cleavlandii. Plant are grown from seed in a mixture of “Peat-Lite Mix®(Speedling, Inc. Sun City, Fla.) and Nutricote® controlled release fertilizer 14-14-14 (Chiss-Asahi Fertilizer Co., Tokyo, Japan). Plants are grown in a controlled environment provided 16 hours of light and 8 hours of darkness. Sylvania “Gro-Lux/Aquarium” wide spectrum 40 watt flourescent grow lights (Osram Sylvania Products, Inc. Danvers, Mass.) are used. Temperatures are kept at around 80° F. during light hours and 70° F. during dark hours. Humidity is between 60 and 85%.

To prepare a DNA insert comprising a nucleic acid sequence of a donor organism, the first step is to construct a cDNA library, a genomic DNA library, or a pool of RNA of the donor organism. Full-length cDNAs or genomic DNA can be obtained from public or private repositories. For example, cDNA and genomic libraries from bovine, chicken, dog, drosophila, fish, frog, human, mouse, porcine, rabbit, rat, and yeast; and retroviral libraries can be obtained from Clontech (Palo Alto, Calif.). Alternatively, cDNA library can be prepared from a field sample by methods known to a person of ordinary skill, for example, isolating mRNAs and transcribing mRNAs into cDNAs by reverse transcriptase (see, e.g., Sambrook et al., Molecular Cloning: A Laboratory Manual (2nd ed.), Vols. 1-3, Cold Spring Harbor Laboratory, (1989), or Current Protocols in Molecular Biology, F. Ausubel et al., ed. Greene Publishing and Wiley-Interscience, New York (1987)). Genomic DNAs represented in BAC (bacterial artificial chromosome), YAC (yeast artificial chromosome), or TAC (transformation-competent artificial chromosome, Lin et al., Proc. Natl. Acad. Sci. USA, 96:6535-6540 (1999)) libraries can be obtained from public or private repositories. Alternatively, a pool of genes, which are overexpressed in a tumor cell line compared with a normal cell line, can be prepared or obtained from public or private repositories. Zhang et al (Science, 276: 1268-1272 (1997)) report that using a method of serial analysis of gene expression (SAGE) (Velculescu et al, Cell, 88:243 (1997)), 500 transcripts that were expressed at significantly different levels in normal and neoplastic cells were identified. The expression of DNAs that overexpresses in a tumor cell line in a host plant may cause changes in the host plant, thus a pool of such DNAs is another source for DNA inserts for this invention. The BAC/YAC/TAC DNAs, DNAs or cDNAs can be mechanically size-fractionated or digested by an enzyme to smaller fragments. The fragments are ligated to adapters with cohesive ends, and shotgun-cloned into recombinant viral nucleic acid vectors. Alternatively, the fragments can be blunt-end ligated into recombinant viral nucleic acid vectors. Recombinant plant viral nucleic acids containing a nucleic acid sequence derived from the cDNA library or genomic DNA library is then constructed using conventional techniques. The recombinant viral nucleic acid vectors produced comprise the nucleic acid insert derived from the donor organism. The nucleic acid sequence of the recombinant viral nucleic acid is transcribed as RNA in a host plant; the RNA is capable of regulating the expression of a phenotypic trait by an antisense mechanism. The nucleic acid sequence may also regulate the expression of more than one phenotypic trait. Nucleic acid sequences from Monera, Protista, Fungi, and Animalia may be used to assemble the DNA libraries. This method may thus be used to discover useful dominant gene phenotypes from DNA libraries through the gene expression in a host plant.

An alternative when creating the recombinant plant viral nucleic acid is to prepare more than one nucleic acid (i.e., to prepare the nucleic acids necessary for a multipartite viral vector construct). In this case, each nucleic acid would require its own origin of assembly. Each nucleic acid could be prepared to contain a subgenomic promoter and a non-native nucleic acid.

In some embodiments of the instant invention, methods to increase the representation of gene sequences in virus expression libraries may also be achieved by bypassing the genetic bottleneck of propagation in bacterial cells. For example, cell-free methods may be used to assemble sequence libraries or individual arrayed sequences into virus expression vectors and reconstruct an infectious virus, such that the final ligation product can be transcribed and the resulting RNA can be used for plant, plant tissue or plant cell inoculation/infection. A more detailed discussion is presented in a co-pending/co-owned U.S. patent application Ser. No. 09/359,303 (Padgett et al., Attorney Docket No. 08010137US03, filed Jul. 21, 1999), enclosed herein by reference.

The host plant can be infected with a recombinant viral nucleic acid or a recombinant plant virus by conventional techniques. Suitable techniques include, but are not limited to, leaf abrasion, abrasion in solution, high velocity water spray, and other injury of a host as well as imbibing host seeds with water containing the recombinant viral RNA or recombinant plant virus. More specifically, suitable techniques include:

(a) Hand Inoculations. Hand inoculations are performed using a neutral pH, low molarity phosphate buffer, with the addition of celite or carborundum (usually about 1%). One to four drops of the preparation is put onto the upper surface of a leaf and gently rubbed.

(b) Mechanized Inoculations of Plant Beds. Plant bed inoculations are performed by spraying (gas-propelled) the vector solution into a tractor-driven mower while cutting the leaves. Alternatively, the plant bed is mowed and the vector solution sprayed immediately onto the cut leaves.

(c) Vacuum Infiltration. Inoculations may be accomplished by subjecting a host organism to a substantially vacuum pressure environment in order to facilitate infection.

(d) High Speed Robotics Inoculation. Especially applicable when the organism is a plant, individual organisms may be grown in mass array such as in microtiter plates. Machinery such as robotics may then be used to transfer the nucleic acid of interest.

(e) High Pressure Spray of Single Leaves. Single plant inoculations can also be performed by spraying the leaves with a narrow, directed spray (50 psi, 6-12 inches from the leaf) containing approximately 1% carborundum in the buffered vector solution.

(f) Ballistics Inoculation. Single plant inoculations can also be performed by particle bombardment. A ballistics particle delivery system (BioRad Laboratories, Hercules, (A) can be used to transfect plants such as N. benthamiana as described previously (Nagar et al., Plant Cell, 7:705-719 (1995)).

An alternative method for introducing a recombinant plant viral nucleic acid into a plant host is a technique known as agroinfection or Agrobacterium-mediated transformation (sometimes called Agro-infection) as described by Grimsley et al., Nature 325:177 (1987), and Turpen et al (J. Virol. Methods, 42:227-240 (1993)). This technique makes use of a common feature of Agrobacterium which colonizes plants by transferring a portion of their DNA (the T-DNA) into a host cell, where it becomes integrated into nuclear DNA. The T-DNA is defined by border sequences which are 25 base pairs long, and any DNA between these border sequences is transferred to the plant cells as well. The insertion of a recombinant plant viral nucleic acid between the T-DNA border sequences results in transfer of the recombinant plant viral nucleic acid to the plant cells, where the recombinant plant viral nucleic acid is replicated, and then spreads systemically through the plant. Agro-infection has been accomplished with potato spindle tuber viroid (PSTV) (Gardner et al., Plant Mol. Biol. 6:221 (1986); CaV (Grimsley et al., Proc. Natl. Acad. Sci. USA 83:3282 (1986)); MSV (Grimsley et al., Nature 325:177 (1987)), and Lazarowitz, S., Nuc. Acids Res. 16:229 (1988)) digitaria streak virus (Donson et al., Virology 162:248 (1988)), wheat dwarf virus (Hayes et al., J. Gen. Virol. 69:891 (1988)) and tomato golden mosaic virus (TGMV) (Elmer et al., Plant Mol. Biol. 10:225 (1988) and Gardiner et al., EMBO J. 7:899 (1988)). Therefore, agro-infection of a susceptible plant could be accomplished with a virion containing a recombinant plant viral nucleic acid based on the nucleotide sequence of any of the above viruses. Particle bombardment or electrosporation or any other methods known in the art may also be used.

Infection may also be attained by placing a selected nucleic acid sequence into an organism such as E. coli, or yeast, either integrated into the genome of such organism or not, and then applying the organism to the surface of the host organism. Such a mechanism may thereby produce secondary transfer of the selected nucleic acid sequence into a host organism. This is a particularly practical embodiment when the host organism is a plant. Likewise, infection may be attained by first packaging a selected nucleic acid sequence in a pseudovirus. Such a method is described in WO 94/10329, the teachings of which are incorporated herein by reference. Though the teachings of this reference may be specific for bacteria, those of skill in the art will readily appreciate that the same procedures could easily be adapted to other organisms.

After a host is infected with a recombinant viral nucleic acid comprising a nucleic acid insert derived from a cDNA library or a genomic library, one or more biochemical or phenotypic changes in a host plant is determined. The biochemical or phenotypic changes in the infected host plant is correlated to the biochemistry or phenotype of a host plant that is uninfected. Optionally, the biochemical or phenotypic changes in the infected host plant is further correlated to a host plant that is infected with a viral vector that contains a control nucleic acid of a known sequence in an antisense orientation; the control nucleic acid has similar size but is different in sequence from the nucleic acid insert derived from the library. For example, if the nucleic acid insert derived from the library is identified as encoding a GTP binding protein in an antisense orientation, a nucleic acid derived from a gene encoding green fluorescent protein can be used as a control nucleic acid. Green fluorescent protein is known not be have the same effect as the GTP binding protein when expressed in plants.

Those of skill in the art will readily understand that there are many methods to determine phenotypic or biochemical change in a plant and to determine the function of a nucleic acid, once the nucleic acid is localized or systemic expressed in a host plant. In a preferred embodiment, the phenotypic or biochemical trait may be determined by observing phenotypic changes in a host by methods including visual, morphological, macroscopic or microscopic analysis. For example, growth change such as stunting, hyperbranching, and necrosis; structure change such as vein banding, ring spot, etching; color change such as bleaching, chlorosis, or other color; and other changes such as marginal, mottled, patterning, punctate, and reticulate are easily detected. In another embodiment, the phenotypic or biochemical trait may be determined by complementation analysis, that is, by observing the endogenous gene or genes whose function is replaced or augmented by introducing the nucleic acid of interest. A discussion of such phenomenon is provided by Napoli et al., The Plant Cell 2:279-289 (1990). In a third embodiment, the phenotypic or biochemical trait may be determined by analyzing the biochemical alterations in the accumulation of substrates or products from enzymatic reactions according to any means known by those skilled in the art. In a fourth embodiment, the phenotypic or biochemical trait may be determined by observing any changes in biochemical pathways which may be modified in a host plant as a result of expression of the nucleic acid. In a fifth embodiment, the phenotypic or biochemical trait may be determined utilizing techniques known by those skilled in the art to observe inhibition of endogenous gene expression in the cytoplasm of cells as a result of expression of the nucleic acid. In a sixth embodiment, the phenotypic or biochemical trait may be determined utilizing techniques known by those skilled in the art to observe changes in the RNA or protein profile as a result of expression of the nucleic acid. In a seventh embodiment, the phenotypic or biochemical trait may be determined by selection of organisms such as plants capable of growing or maintaining viability in the presence of noxious or toxic substances, such as, for example herbicides and pharmaceutical ingredients.

Phenotypic traits in plant cells, which may be observed microscopically, macroscopically or by other methods, include improved tolerance to herbicides, improved tolerance to extremes of heat or cold, drought, salinity or osmotic stress; improved resistance to pests (insects, nematodes or arachnids) or diseases (fungal, bacterial or viral), production of enzymes or secondary metabolites; male or female-sterility; dwarfness; early maturity; improved yield, vigor, heterosis, nutritional qualities, flavor or processing properties, and the like. Other examples include the production of important proteins or other products for commercial use, such as lipase, melanin, pigments, alkaloids, antibodies, hormones, pharmaceuticals, antibiotics and the like. Another useful phenotypic trait is the production of degradative or inhibitory enzymes, for example, enzymes preventing or inhibiting the root development in malting barley, or enzymes determining response or non-response to a systemically administered drug in a human. The phenotypic trait may also be a secondary metabolite whose production is desired in a bioreactor.

Biochemical changes can also be determined by analytical methods, for example, in a high-throughput, fully automated fashion using robotics. Suitable biochemical analysis may include MALDI-TOF, LC/MS, GC/MS, two-dimensional IEF/SDS-PAGE, ELISA or other methods of analyses. The clones in the plant viral vector library may then be functionally classified based on metabolic pathway affected or visual/selectable phenotype produced in the plant. This process enables a rapid determination of gene function for unknown nucleic acid sequences of a donor organism as well as a plant origin. Furthermore, this process can be used to rapidly confirm function of full-length DNA's of unknown function. Functional identification of unknown nucleic acid sequences in a library of one organism may then rapidly lead to identification of similar unknown sequences in expression libraries for other organisms based on sequence homology. Such information is useful in many aspects including human medicine.

One useful means to determine the function of nucleic acids transfected into a host is to observe the effects of gene silencing. Traditionally, functional gene knockout has been achieved following inactivation due to insertion of transposable elements or random integration of T-DNA into the chromosome, followed by characterization of conditional, homozygous-recessive mutants obtained upon backcrossing. Some teachings in these regards are provided by WO 97/42210 which is herein incorporated by reference. As an alternative to traditional knockout analysis, an EST/DNA library from a donor organism, may be assembled into a plant viral transcription plasmid. The nucleic acid sequences in the transcription plasmid library may then be introduced into plant cells as part of a functional RNA virus which post-transcriptionally silences the homologous target gene. The EST/DNA sequences may be introduced into a plant viral vector in either the plus or minus sense orientation, and the orientation can be either directed or random based on the cloning strategy. A high-throughput, automated cloning scheme based on robotics may be used to assemble and characterize the library. Alternatively, the EST/cDNA sequences can be inserted into the genomic RNA of a plant viral vector such that they are represented as genomic RNA during the viral replication in plant cells. The library of EST clones is then transcribed into infectious RNAs and inoculated onto a host plant susceptible to viral infection. The viral RNAs containing the EST/cDNA sequences contributed from the original library are now present in a sufficiently high concentration in the cytoplasm of host plant cells such that they cause post-transcriptional gene silencing of the endogenous gene in a host plant. Since the replication mechanism of the virus produces both sense and antisense RNA sequences, the orientation of the EST/cDNA insert is normally irrelevant in terms of producing the desired phenotype in the host plant.

The present invention provides a method to express transiently viral-derived antisense RNAs in transfected plants. Such method is much faster than the time required to obtain genetically engineered antisense transgenic plants. Systemic infection and expression of viral antisense RNA occurs as short as four days post inoculation, whereas it takes several months or longer to create a single transgenic plant. The invention provides a method to identify genes involved in the regulation of plant growth by inhibiting the expression of specific endogenous genes using viral vectors. This invention provides a method to characterize specific genes and biochemical pathways in donor organisms or in host plants using an RNA viral vector.

One problem with gene silencing in a plant host is that many plant genes exist in multigene families. Therefore, effective silencing of a gene function may be especially problematic. According to the present invention, however, nucleic acids may be inserted into the viral genome to effectively silence a particular gene function or to silence the function of a multigene family. It is presently believed that about 20% of plant genes exist in multigene families.

A detailed discussion of some aspects of the “gene silencing” effect is provided in the co-pending patent application, U.S. patent application Ser. No. 08/260,546 (WO 95/34668 published Dec. 21, 1995) the disclosure of which is incorporated herein by reference. RNA can reduce the expression of a target gene through inhibitory RNA interactions with target mRNA that occur in the cytoplasm and/or the nucleus of a cell.

It is known that silencing of endogenous genes can be achieved with homologous sequences from the same family. For example, Kumagai et al., (Proc. Natl. Acad. Sci. USA 92:1679 (1995)) report that the Nicotiana benthamiana gene for phytoene desaturase (PDS) was silenced by transfection with a viral RNA derived from a clone containing a partial tomato (Lycopersicon esculentum) cDNA encoding PDS being in an antisense orientation. This paper is incorporated here by reference. Kumagai et al. demonstrate that gene encoding PDS from one plant can be silenced by transfecting a host plant with a nucleic acid of a known sequence, namely, a PDS gene, from a donor plant of the same family. The present invention provides a method of silencing a gene in a host plant by transfecting the host plant with a viral nucleic acid comprising a nucleic acid insert derived from a cDNA library or a genomic DNA library or a pool of RNA from a non-plant organism. Different from Kumagai et al, the sequence of the nucleic acid insert in the present invention does not need to be identified prior to the transfection. Another feature of the present invention is that it provides a method to silence a conserved gene of a different kingdom; the antisense transcript of a non-plant organism results in reducing expression of the endogenous gene or multigene family of a plant. The invention is exemplified by GTP binding proteins. In eukaryotic cells, GTP-binding proteins function in a variety of cellular processes, including signal transduction, cytoskeletal organization, and protein transport. Low molecular weight (20-25 K Daltons) of GTP-binding proteins include ras and its close relatives (for example, Ran), rho and its close close relatives, the rab family, and the ADP-ribosylation factor (ARF) family. The heterotrimeric and monomeric GTP-bifiding proteins that may be involved in secretion and intracellular transport are divided into two structural classes: the rab and the ARF families. The ARFs from many organisms have been isolated and characterized. The ARFs share structural features with both the ras and trimeric GTP-binding protein families. The present invention demonstrates that genes of one plant, such as Nicotiana, which encode GTP binding proteins, can be silenced by transfection with infectious RNAs from a clone containing GTP binding protein open reading frame in an antisense orientation, derived from a plant of a different family, such as Arabidopsis. The present invention also demonstrates that a plant GTP binding protein is highly homologous to the GTP binding proteins from a non-plant organism such as a human, a frog, a mouse, a bovine, a fly and a yeast, not only at the amino acid level, but also at the nucleic acid level. The present invention thus provides a method to 'silence a conserved gene in a host plant, by transfecting the plant with infectious RNAs derived from a homologous gene of a non-plant organism.

The invention is also directed to a method of determining a nucleic acid sequence in a donor organism from Monera, Protisca, Fungi and Animalia, which has the same function as that in a host organism, by transfecting a nucleic acid sequence derived from a donor organism into a host. In one preferred embodiment, the method comprising the steps of (a) preparing a library of cDNAs, or a genomic DNAs or a pool of RNAs of the donor organism, (b) constructing recombinant viral nucleic acids comprising a nucleic acid insert derived from the library, (c) infecting each host with one of the recombinant viral nucleic acids, (d) growing the infected host, (e) detecting one or more changes in the host, (f) identifying the recombinant viral nucleic acid that results in changes in the host, (g) determining the sequence of the nucleic acid insert in the recombinant viral nucleic acid that results in changes in the host, and (h) determining the sequence of an entire open reading frame of the donor from which the nucleic acid insert is derived.

The invention is further directed to a method of determining a nucleic acid sequence in a host plant, which has the same function or has homology as that in a donor organism from Monera, Protisca, Fungi and Animalia, by transfecting a nucleic acid sequence derived from a donor organism into a host. In one preferred embodiment, the method comprising the steps of (a) preparing a cDNA library, a genomic DNA library, or a mRNA pool of the donor organism, (b) constructing recombinant viral nucleic acids comprising a nucleic acid insert derived from the library, (c) infecting each host with one of the recombinant viral nucleic acids, (d) growing the infected host, (e) detecting one or more changes in the host, (f) identifying the recombinant viral nucleic acid that results in changes in the host, (g) determining the sequence of the nucleic acid insert in the recombinant viral nucleic acid that results in changes in the host, and (h) determining the sequence of an entire open reading frame of a gene in the host plant, the expression of which is affected by the insert. The sequence of the nucleic acid insert in the cDNA clone or in the viral vector can be determined by a standard method, for example, by dideoxy termination using double stranded templates (Sanger et al., Proc., Natl. Acad. Sci. USA 74:5463-5467 (1977)). Once the sequence of the nucleic acid insert is obtained, the sequence of an entire open reading frame of a gene can be determined by probing filters containing full-length cDNAs from the cDNA library with the nucleic acid insert labeled with radioactive, fluorescent, or enzyme molecules. The sequence of an entire open reading frame of a gene can also be determined by RT-PCR (Methods Mol. Biol. 89:333-358 (1998)).

The present invention also provides a method of isolating a conserved gene from a donor organism such as Monera, Protisca, Fungi or Animalia. Libraries containing full-length cDNAs from fungi, and animals can be obtained from public and private sources or can be prepared from mRNAs. The cDNAs are inserted in viral vectors or in small subeloning vectors such as pBluescript (Strategene), pUC18, M13, or pBR322. Transformed bacteria are then plated and individual clones selected by a standard method. The bacteria transformants or DNAs are rearrayed at high density onto membrane filters or glass slides. Full-length cDNAs can be identified by probing filters or slides with labeled nucleic acid inserts which result in changes in a host plant. Useful labels include radioactive, fluorescent, or chemiluminecent molecules, enzymes, etc. For example, the present invention is directed to a method of isolating human cDNA, comprising the steps of: (a) obtaining a cDNA library from a human organism, (b) constructing recombinant viral nucleic acids comprising a nucleic acid insert derived from said library, (c) infecting a host plant with said recombinant viral nucleic acids, and expressing transiently said nucleic acid in an antisense orientation in said host plant, (d) growing said infected host plant, (e) detecting one or more changes in said host plant, (f) identifying said recombinant viral nucleic acid that results in changes in said host plant, (g) sequencing and labeling said nucleic acid insert in said recombinant viral nucleic acid of (f), (h) probing filters or slides containing full-length human cDNAs with said labeled nucleic acid insert, and (i) isolating said full-length human cDNA that hybridizes to said labeled nucleic acid insert.

Alternatively, genomic libraries containing sequences from fungi, animals and libraries from retroviruses can be obtained from public and private sources, or be prepared from plant genomic DNAs. BAC clones containing entire plant genomes have been constructed and organized in a minimal overlapping order. Individual BACs are sheared to fragments and directly cloned into viral vectors. Clones that completely cover an entire BAC form a BAC viral vector sublibrary. Genomic clones can be identified by probing filters containing BACs with labeled nucleic acid inserts which result in changes in a host plant. Useful labels include radioactive, fluorescent, or chemiluminecent molecules, enzymes, etc. BACs that hybridize to the probe are selected and their corresponding BAC viral vectors are used to produce infectious RNAs. Plants that are transfected with the BAC sublibrary are screened for change of function, for example, change of growth rate or change of color. Once the change of function is observed, the inserts from these clones or their corresponding plasmid DNAs are characterized by dideoxy sequencing. This provides a rapid method to obtain the genomic sequence of a donor organism. Using this method, once the DNA sequence in one organism is identified, it can be used to identify conserved sequences of similar function that exist in other libraries. This method speeds up the rate of discovering new genes.

The present invention provides a method to produce a non-plant protein in a plant. After DNAs of similar functions from a plant and a non-plant organism are isolated and identified, the amino acid sequences derived from the DNAs are compared. The plant DNA sequence is changed so that it encodes the same amino acid sequence as the DNA of the non-plant organism encodes. The DNA sequence can be changed according to methods known to an ordinary skilled person, for example, site directed mutagenesis or DNA synthesis. One aspect of the invention is to provide a method of humanizing a plant cDNA. The method comprises selecting a plant cDNA that is homologous to human cDNA and making changes of the plant DNA, so that the modified plant cDNA expresses a human protein in a plant host. The production of such human protein may be used in human medicine.

Nucleic acid sequences that may result in changing a plant phenotype include those involved in cell growth, proliferation, differentiation and development; cell communication; and the apoptotic pathway. Genes regulating growth of cells or organisms include, for example, genes encoding a GTP binding protein, a ribosomal protein L19 protein, an SI18 ribosomal protein, etc. Henry et al. (Cancer Res., 53:1403-1408 (1993)) report that erb B-2 (or HER-2 or neu) gene was amplied and overexpressed in one-third of cancers of the breast, stomach, and overy; and the mRNA encoding the ribosomal protein L19 was more abundant in breast cancer samples that express high levels of erbB-2. Lijsebettens et al. (EMBO J, 13:3378-3388 (1994)) report that in Arabidopsis, mutation at PFL caused pointed first leaves, reduced fresh weight and growth retardation. PFL codes for ribosomal protein S18, which has a high homology with the rat S18 protein. Genes involved in development of cells or organisms include, for example, homeobox-containing genes and genes encoding G-protein-coupled receptor proteins such as the rhodopsin family. Homeobox genes are a family of regulatory genes containing a common 183-nucleotide sequence (homeobox) and coding for specific nuclear proteins (homeoproteins) that act as transcription factors. The homeobox sequence itself encodes a 61-amino-acid domain, the homeodomain, responsible for recognition and binding of sequence-specific DNA motifs. The specificity of this binding allows homeoproteins to activate or repress the expression of batteries of down-stream target genes. Initially identified in genes controlling Drosophila development, the homeobox has subsequently been isolated in evolutionarily distant animal species, plants, and fungi. Several indications suggest the involvement of homeobox genes in the control of cell growth and, when dysregulated, in oncogenesis (Cillo et al., Exp. Cell Res., 248:1-9 (1999). Other nucleic acid sequences that may result in changes of a plant include genes encoding receptor proteins such as hormone receptors, cAMP receptors, serotonin receptors, and calcitonin family of receptors; and light-regulated DNA encoding a leucine (Leu) zipper motif (Zheng et al., Plant Physiol., 116:27-35 (1998)). Deregulation or alteration of the process of cell growth, proliferation, differentiation and development; cell communication; and the apoptotic pathways may result in cancer. Therefore, identifying the nucleic acid sequences involved in those processes and determining their functions are beneficial to the human medicine; it also provides a tool for cancer research.

Large amounts of DNA sequence information are being generated in the public domain, which may be entered into a relational database. Links may be made between sequences from various species predicted to carry out similar biochemical or regulatory functions. Links may also be generated between predicted enzymatic activities and visually displayed biochemical and regulatory pathways. Likewise, links may be generated between predicted enzymatic or regulatory activity and known small molecule inhibitors, activators, substrates or substrate analogs. Phenotypic data from expression libraries expressed in transfected hosts may be automatically linked within such a relational database. Genes with similar predicted roles of interest in other organisms may be rapidly discovered.

A complete classification scheme of gene functionality for a fully sequenced eukaryotic organism has been established for yeast. This classification scheme may be modified for other organisms and divided into the appropriate categories. Such organizational structure may be utilized to rapidly identify herbicide target loci which may confer dominant lethal phenotypes, and thereby is useful in helping to design rational herbicide programs.

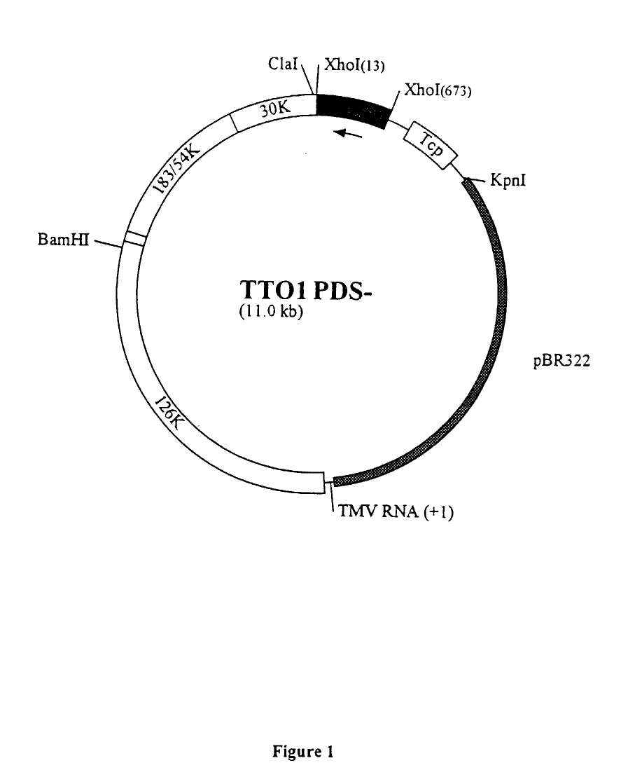

This invention is exemplified by setting up a functional genomics screen using a Tobacco Mosaic Virus having a TMV-0 coat protein capsid for infection of Nicotiana benthamiana, a plant related to the common tobacco plant. A human cDNA library is obtained from Clontech laboratories (Palo Alto, Calif.) on a “bacteria artificial chromosomes:” (BAC). The BACs are further subdivided into viral vector clones by inserting a section of cDNA at the 3′ end of a subgenomic promoter of the viral vector. The inserts are made in the antisense orientation as in FIG. 1 until all of the cDNA from the BAC human cDNA library is represented on viral vectors. Each viral vector is sprayed onto the leaf of a 2 week old N. benthamiana plant with sufficient force to cause tissue injury and localized infection. Each infected plant is grown side by side with an uninfected plant and a plant infected with a null insert vector as control. All plants are grown in an artificial environment having 16 hours of light and 8 hours of dark. Lumens are approximately equal on each plant. At intervals of 2 days, a visual and photographic observation of phenotype is made and recorded for each infected plant and each of its controls and a comparison is made. Data is entered into a Laboratory Information Management System database. At the end of the observation period severly stunted plants, for example, are grouped for analysis. The nucleic acid insert contained in the viral vector clone 740AT #2483 is responsible for severe stunting of one of the plants. Clone 740AT #2483 is sequenced. The homolog from the plant host is also sequenced. The 740AT-#2483 clone is found to have 71% homology to the plant host nucleic acid sequence. The protein sequence homology is 83%. The entire human cDNA sequence from which the insert was derived is obtained by sequencing and found to code for human ribosomal protein L19 S56985. The host plant homolog is selected and sequenced. It also codes for a ribosomal protein. We conclude that this ribosomal coding sequence is highly conserved in nature. This information is useful in pharmaceutical development as well as in toxicology studies.

The present invention is also directed to a method of increasing yield of a grain crop. In Rice Biotechnology Quarterly (37:4, (1999)), it is reported that a transgenic rice plant transformed with a rgpI gene, which encodes a small GTP binding protein from rice, was shorter than a control plant, but it produced more seeds than the control plant. To increase the yield of a grain crop, the present method comprises expressing a nucleic acid sequence of a non-plant organism in an antisense orientation in the cytoplasm of the grain crop, wherein said expressing results in stunted growth and increased seed production of said grain crop. A preferred method comprises the steps of cloning the nucleic acid sequence into a plant viral vector and infecting the grain crop with a recombinant viral nucleic acid comprising said nucleic acid sequence. Preferred plant viral vector is derived from a Brome Mosaic virus, a Rice Necrosis virus, or a geminivirus. Preferred grain crops include rice, wheat, and barley. The nucleic acid expressed in the host plant, for example, comprises a GTP binding protein open reading frame having an antisense orientation. The present method provides a transiently expression of a gene to obtain a stunted plant. Because less energy is put into plant growth, more energy is available for production of seed, which results in increase yield of a grain crop. The present method has an advantage over other method using a trangenic plant, because it does not have an effect on the genome of a host plant.

In order to provide an even clearer and more consistent understanding of the specification and the claims, including the scope given herein to such terms, the following definitions are provided:

Adjacent: A position in a nucleotide sequence proximate to and 5′ or 3′ to a defined sequence. Generally, adjacent means within 2 or 3 nucleotides of the site of reference.

Anti-Sense Inhibition: A type of gene regulation based on cytoplasmic, nuclear or organelle inhibition of gene expression due to the presence in a cell of an RNA molecule complementary to at least a portion of the mRNA being translated. It is specifically contemplated that RNA molecules may be from either an RNA virus or mRNA from the host cells genome or from a DNA virus.

Cell Culture: A proliferating group of cells which may be in either an undifferentiated or differentiated state, growing contiguously or non-contiguously.

Chimeric Sequence or Gene: A nucleotide sequence derived from at least two heterologous parts. The sequence may comprise DNA or RNA.

Coding Sequence: A deoxyribonucleotide or ribonucleotide sequence which, when either transcribed and translated or simply translated, results in the formation of a cellular polypeptide or a ribonucleotide sequence which, when translated, results in the formation of a cellular polypeptide.

Compatible: The capability of operating with other components of a system. A vector or plant or animal viral nucleic acid which is compatible with a host is one which is capable of replicating in that host. A coat protein which is compatible with a viral nucleotide sequence is one capable of encapsidating that viral sequence.

Complementation Analysis: As used herein, this term refers to observing the changes produced in an organism when a nucleic acid sequence is introduced into that organism after a selected gene has been deleted or mutated so that it no longer functions fully in its normal role. A complementary gene to the deleted or mutated gene can restore the genetic phenotype of the selected gene.

Dual Heterologous Subgenomic Promoter Expression System (DHSPES): a plus stranded RNA vector having a dual heterologous subgenomic promoter expression system to increase, decrease, or change the expression of proteins, peptides or RNAs, preferably those described in U.S. Pat. Nos. 5,316,931, 5,811,653, 5,589,367, and 5,866,785, the disclosure of which is incorporated herein by reference.

Expressed sequence tags (ESTs): Relatively short single-pass DNA sequences obtained from one or more ends of cDNA clones and RNA derived therefrom. They may be present in either the 5′ or the 3′ orientation. ESTs have been shown useful for identifying particular genes.

Expression: The term as used herein is meant to incorporate one or more of transcription, reverse transcription and translation.

A functional Gene Profile: The collection of genes of an organism which code for a biochemical or phenotypic trait. The functional gene profile of an organism is found by screening nucleic acid sequences from a donor organism by over expression or suppression of a gene in a host organism. A functional gene profile requires a collection or library of nucleic acid sequences from a donor organism. A functional gene profile will depend on the ability of the collection or library of donor nucleic acids to cause over-expression or suppression in the host organism. Therefore, a functional gene profile will depend upon the quantity of donor genes capable of causing over-expression or suppression of host genes or of being expressed in the host organism in the absence of a homologous host gene.

Gene: A discrete nucleic acid sequence responsible for producing one or more cellular products and/or performing one or more intercellular or intracellular functions.

Gene silencing: A reduction in gene expression. A viral vector expressing gene sequences from a host may induce gene silencing of homologous gene sequences.

Homology: A degree of nucleic acid similarity in all or some portions of a gene sequence sufficient to result in gene suppression when the nucleic acid sequence is delivered in the antisense orientation.

Host: A cell, tissue or organism capable of replicating a nucleic acid such as a vector or plant viral nucleic acid and which is capable of being infected by a virus containing the viral vector or viral nucleic acid. This term is intended to include prokaryotic and eukaryotic cells, organs, tissues or organisms, where appropriate. Bacteria, fungi, yeast, animal (cell, tissues, or organisms), and plant (cell, tissues, or organisms) are examples of a host.

Infection: The ability of a virus to transfer its nucleic acid to a host or introduce a viral nucleic acid into a host, wherein the viral nucleic acid is replicated, viral proteins are synthesized, and new viral particles assembled. In this context, the terms “transmissible” and “infective” are used interchangeably herein. The term is also meant to include the ability of a selected nucleic acid sequence to integrate into a genome, chromosome or gene of a target organism.

Multigene family: A set of genes descended by duplication and variation from some ancestral gene. Such genes may be clustered together on the same chromosome or dispersed on different chromosomes. Examples of multigene families include those which encode the histones, hemoglobins, immunoglobulins, histocompatibility antigens, actins, tubulins, keratins, collagens, heat shock proteins, salivary glue proteins, chorion proteins, cuticle proteins, yolk proteins, and phaseolins.

Non-Native: Any RNA or DNA sequence that does not normally occur in the cell or organism in which it is placed. Examples include recombinant plant viral nucleic acids and genes or ESTs contained therein. That is, an RNA or DNA sequence may be non-native with respect to a viral nucleic acid. Such an RNA or DNA sequence would not naturally occur in the viral nucleic acid. Also, an RNA or DNA sequence may be non-native with respect to a host organism. That is, such a RNA or DNA sequence would not naturally occur in the host organism.

Nucleic acid: As used herein the term is meant to include any DNA or RNA sequence from the size of one or more nucleotides up to and including a complete gene sequence. The term is intended to encompass all nucleic acids whether naturally occurring in a particular cell or organism or non-naturally occurring in a particular cell or organism. Nucleic acid of interest: The term is intended to refer to the nucleic acid sequence whose function is to be determined. The sequence will normally be non-native to a viral vector but may be native or non-native to a host organism.

Phenotypic Trait: An observable, measurable or detectable property resulting from the expression or suppression of a gene or genes.

Plant Cell: The structural and physiological unit of plants, consisting of a protoplast and the cell wall.

Plant Organ: A distinct and visibly differentiated part of a plant, such as root, stem, leaf or embryo.

Plant Tissue: Any tissue of a plant in planta or in culture. This term is intended to include a whole plant, plant cell, plant organ, protoplast, cell culture, or any group of plant cells organized into a structural and functional unit.

Positive-sense inhibition: A type of gene regulation based on cytoplasmic inhibition of gene expression due to the presence in a cell of an RNA molecule substantially homologous to at least a portion of the mRNA being translated.

Promoter: The 5′-flanking, non-coding sequence substantially adjacent a coding sequence which is involved in the initiation of transcription of the coding sequence.

Protoplast: An isolated plant or bacterial cell without some or all of its cell wall.

Recombinant Plant Viral Nucleic Acid: Plant viral nucleic acid which has been modified to contain non-native nucleic acid sequences. These non-native nucleic acid sequences may be from any organism or purely synthetic, however, they may also include nucleic acid sequences naturally occurring in the organism into which the recombinant plant viral nucleic acid is to be introduced.

Recombinant Plant Virus: A plant virus containing the recombinant plant viral nucleic acid.

Subgenomic Promoter: A promoter of a subgenomic mRNA of a viral nucleic acid.

Substantial Sequence Homology: Denotes nucleotide sequences that are substantially functionally equivalent to one another. Nucleotide differences between such sequences having substantial sequence homology are insignificant in affecting function of the gene products or an RNA coded for by such sequence.

Systemic Infection: Denotes infection throughout a substantial part of an organism including mechanisms of spread other than mere direct cell inoculation but rather including transport from one infected cell to additional cells either nearby or distant.

Transposon: A nucleotide sequence such as a DNA or RNA sequence which is capable of transferring location or moving within a gene, a chromosome or a genome.

Transgenic plant: A plant which contains a foreign nucleotide sequence inserted into either its nuclear genome or organellar genome.

Transcription: Production of an RNA molecule by RNA polymerase as a complementary copy of a DNA sequence or subgenomic mRNA.

Transient expression: Expression of a nucleic acid sequence in a host without insertion of the nucleic acid sequence into the host genome, such as by way of a viral vector.

Vector: A self-replicating RNA or DNA molecule which transfers an RNA or DNA segment between cells, such as bacteria, yeast, plant, or animal cells.

Virus: An infectious agent composed of a nucleic acid which may or may not be encapsidated in a protein. A virus may be a mono-, di-, tri-, or multi-partite virus, as described above.

### EXAMPLES

The following examples further illustrate the present invention. These examples are intended merely to be illustrative of the present invention and are not to be construed as being limiting.

### Example 1

Gene silencing/co-supression of genes induced by delivering an RNA capable of base pairing with itself to form double stranded regions.

Gene silencing has been used to down regulate gene expression in transgenic plants. Recent experimental evidence suggests that double stranded RNA may be an effective stimulator of gene silencing/co-suppression phenomenon in transgenic plant. For example, Waterhouse et al. (Proc. NatL Acad. Sci. USA 95:13959-13964 (1998), incorporated herein by reference) described that virus resistance and gene silencing in plants could be induced by simultaneous expression of sense and antisense RNA. Gene silencing/co-suppression of plant genes may be induced by delivering an RNA capable of base pairing with itself to form double stranded regions.

This example shows: (1) a novel method for generating an RNA virus vector capable of producing an RNA capable of forming double stranded regions, and (2),a process to silence plant genes by using such a viral vector.

Step 1: Construction of a DNA sequence which after it is transcribed would generate an RNA molecule capable of base pairing with itself. Two identical, or nearly identical, ds DNA sequences are ligated together in an inverted orientation to each other (i.e., in either a head to tail or tail to head orientation) with or without a linking nucleotide sequence between the homologous sequences. The resulting DNA sequence is then be cloned into a cDNA copy of a plant viral vector genome.

Step 2: Cloning, screening, transcription of clones of interest using known methods in the art.

Step 3: Infect plant cells with transcripts from clones.

As virus expresses foreign gene sequence, RNA from foreign gene forms base pair upon itself, forming double-stranded RNA regions. This approach is used with any plant or non-plant gene and used to silence plant gene homologous to assist in identification of the function of a particular gene sequence.

### Example 2

Cytoplasmic inhibition of phvtoene desaturase in transfected plant confirms that the partial tomato PDS sequence encodes phvtoene desaturase.

Isolation of tomato mosaic virus cDNA. An 861 base pair fragment (5524-6384) from the tomato mosaic virus (fruit necrosis strain F; tom-F) containing the putative coat protein subgenomic promoter, coat protein gene, and the 3′-end was isolated by PCR using primers 5′-CTCGCAAAGTTTCGAACCAAATCCTC-3′ (upstream) (SEQ ID NO: 1) and 5′-CGGGGTACCTGGGCCCCAACCGGGGGTTCCGGGGG-3′ (downstream) (SEQ ID NO: 2) and subcloned into the HincII site of pBluescript KS-. A hybrid virus consisting of TMV-U1 and ToMV-F was constructed by swapping an 874-bp BamHI-KpnI ToMV fragment into pBGC152, creating plasmid TTO1. The inserted fragment was verified by dideoxynucleotide sequencing. A unique AvrII site was inserted downstream of the XhoI site in TTO1 by PCR mutagenesis, creating plasmid TTO1A, using the following oligonucleotides: 5′-TCCTCGAGCCTAGGCTCGCAAAGTTTCGAACCAAATCCTCA-3′ (upstream) (SEQ ID NO: 3), 5′-CGGGGTACCTGGGCCCCAACCGGGGGTTCCGGGGG-3′ (downstream) (SEQ ID NO: 4).

Isolation of a partial cDNA encoding tomato phytoene desaturase. Partial cDNAs were isolated from ripening tomato fruit RNA by polymerase chain reaction (PCR) using the following oligonucleotides: PDS, 5′-TGCTCGAGTGTGTTCTTCAGTTTTCTGTCA-3′ (SEQ ID NO: 5) (upstream), 5′-AACTCGAGCGCTTTGATTTCTCCGAAGCTT-3′(downstream) (SEQ ID NO: 6). Approximately 3×10⁴ colonies from a Lycopersicon esculentum cDNA library were screened by colony hybridization using a ³²P labeled tomato phytoene desaturase PCR product. Hybridization was carried out at 42° C. for 48 hours in 50% formamide, 5×SSC, 0.02 M phosphate buffer, 5×Denhart's solution, and 0.1 mg/ml sheared calf thymus DNA. Filters were washed at 65° C. in 0.1×SSC, 0.1% SDS prior to autoradiography. PCR products and the phytoene desaturase cDNA clones were verified by dideoxynucleotide sequencing.

DNA sequencing and computer analysis. A PstI, BamHI fragment containing the partial phytoene desaturase cDNA was subcloned into pBluescript® KS+(Stratagene, La Jolla, Calif.). The nucleotide sequencing of KS+/PDS #38 was carried out by dideoxy termination using single-stranded templates (Maniatis, Molecular Cloning, 1^{st} Ed.) Nucleotide sequence analysis and amino acid sequence comparisons were performed using PCGENE® and DNA Inspector® IIE programs.

Construction of a viral vector containing a partial tomato phytoene desaturase cDNA. A XhoI fragment containing the partial tomato phytoene desaturase cDNA was subcloned into TTO 1. The vector TTO1A/PDS+(FIG. 1) contains the phytoene desaturase cDNA in the positive orientation under the control of the TMV-U1 coat protein subgenomic promoter; while the vector TTO1A/PDS—contains the phytoene desaturase cDNA in the antisense orientation.

Analysis of N. benthamiana transfected by TTO1/PDS+, and TTO1/PDS−. Infectious RNAs from TTO1/PDS+, TTO1/PDS+ were prepared by in vitro transcription using SP6 DNA-dependent RNA polymerase as described previously (Dawson et al., Proc. Natl. Acad. Sci. USA 83:1832 (1986)) and were used to mechanically inoculate N. benthamiana. The hybrid viruses spread throughout all the non-inoculated upper leaves as verified by transmission electron microscopy, local lesion infectivity assay, and polymerase chain reaction (PCR) amplification. The viral symptoms resulting from the infection consisted of distortion of systemic leaves and plant stunting with mild chlorosis. The leaves from plants transfected with TTO1/PDS+ and TTO1/PDS− turned white. Agarose gel eletrophoresis of PCR cDNA isolated from virion RNA and Northern blot analysis of virion RNA indicate that the vectors are maintained in an extrachromosomal state and have not undergone any detectable intramolecular rearrangements.

Purification and analysis of carotenoids from transfected plants. The carotenoids were isolated from systemically infected tissue and analyzed by HPLC chromatography. Carotenoids were extracted in ethanol and identified by their peak retention time and absorption spectra on a 25-cm Spherisorb® ODS-15-m column using acetonitrile/methanol/2-propanol (85:10:5) as a developing solvent at a flow rate of 1 ml/min. They had identical retention time to a synthetic phytoene standard and β-carotene standards from carrot and tomato. The expression of sense and antisense RNA to a partial phytoene desaturase in transfected plants inhibited the synthesis of colored carotenoids and caused the systemically infected leaves to turn white. HPLC analysis of these plants revealed that they also accumulated phytoene. The white leaf phenotype was also observed in plants treated with the herbicide norflurazon which specifically inhibits phytoene desaturase.

Our results that phytoene accumulated in plants transfected with antisense phytoene desaturase suggests that viral vectors can be used as a potent tool to manipulate pathways in the production of secondary metabolites through cytoplasmic antisense inhibition. Leaves from systemically infected TTO1A/PDS+ plants also accumulated phytoene and developed a bleaching white phenotype; the actual mechanism of inhibition is not clear. These data are presented by Kumagai et al., Proc. Natl. Acad. Sci. USA 92:1679-1683 (1995).

### Example 3

Cytoplasmic inhibition of 5-enolpyruvylshikimate-3-phosphate synthase (EPSPS) genes in plants blocks aromatic amino acid biosynthesis.

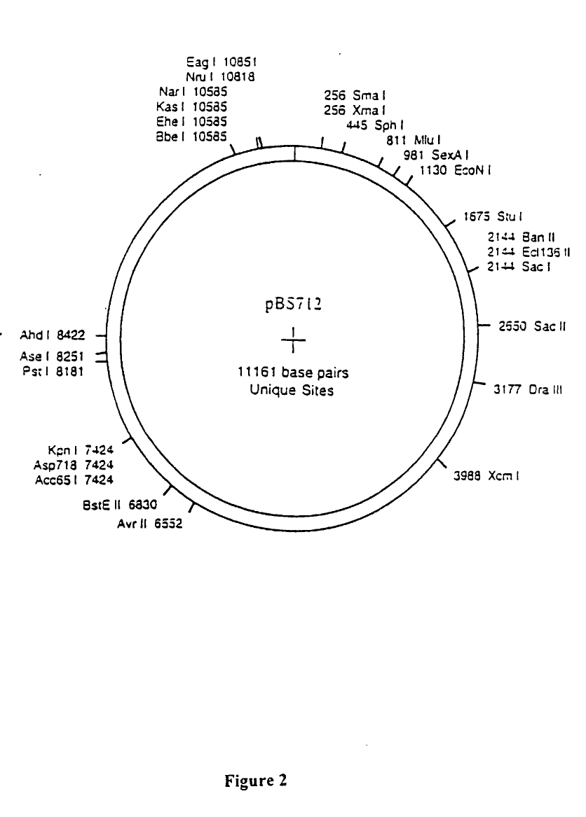

Cytoplasmic inhibition of 5-enolpyruvylshikimate-3-phosphate synthase (EPSPS) genes in plants blocks aromatic amino acid biosynthesis and causes a systemic bleaching phenotype similar to Roundup(® herbicide. See also della-Cioppa, et al., “Genetic Engineering of herbicide resistance in plants,” Frontiers of Chemistry: Biotechnology, Chemical Abstract Service, ACS, Columbus, Ohio, pp. 665-70 (1989). A dual subgenomic promoter vector encoding 1097 base pairs of an antisense EPSPS gene from Nicotiana tabacum (Class I EPSPS) is shown in plasmid pBS712. FIG. 2 shows plasmid pBS712. Systemic expression of the Nicotiana tabacum Class I EPSPS gene in the antisense orientation causes a systemic bleaching phenotype similar to Roundup® herbicide.

### Example 4

Exemplary complementation analysis.

A transgenic plant or naturally occurring plant mutant may have a non-functional gene such as the one which produces EPSPS. A plant deficient or lacking in the EPSPS gene could grow only in the presence of added aromatic amino acids. Transfection of plants with a viral vector containing a functional EPSPS gene or cDNA sequence encoding the same would cause the plant to produce a functional EPSPS gene product. A plant so transfected would then be able to grow normally without added aromatic amino acids to its environment. In this transfected plant, the EPSPS mutation in the plant would be complemented in trans by the viral nucleic acid sequence containing the native or foreign EPSPS cDNA sequence.

### Example 5

Construction of a tobamoviral vector for expression of heterologous genes in A. thaliana.

Virions that were prepared as a crude aqueous extract of tissue from turnip infected with Ribgrass mosaic virus (RMV) were used to inoculate N. benthamiana, N. tabacum, A. thaliana, and oilseed rape (canola). Two to three weeks after transfection, systemically infected plants were analyzed by immunoblotting, using purified RMV as a standard. Total soluble plant protein concentrations were determined using bovine serum albumin as a standard. The proteins were analyzed on a 0.1% SDS/12.5% polyacrylarnide gel and transferred by electroblotting for 1 hr to a nitrocellulose membrane. The blotted membrane was incubated for 1 hr with a 2000-fold dilution of anti-ribgrass mosaic virus coat antiserum. Using standard protocols, the antisera was raised in rabbits against purified RMV coat protein. The enhanced chemiluminescence horseradish peroxidase-linked, goat anti-rabbit IgG assay (Cappel Laboratories) was performed according to the manufacturer's (Amersham) specifications. The blotted membrane was subjected to film exposure times of up to 10 sec. No detectable cross-reacting protein was observed in the noninfected N. benthamiana control plant extracts. A 18 kDa protein cross-reacted to the anti-RMV coat antibody from systemically infected N. benthamiana, N. tabacum, A. thaliana, and oilseed rape (canola). This result demonstrates that RMV can systemically infect N. benthamiana, N. tabacum, A. thaliana, and oilseed rape (canola).

Plasmid constructions.

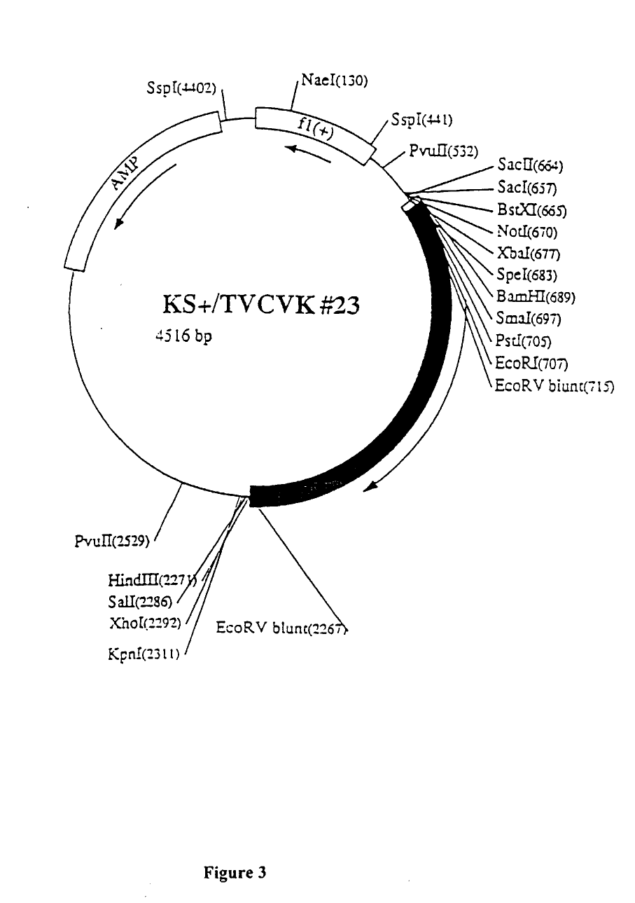

Ribgrass mosaic virus (RMV) is a member of the tobamovirus group that infects crucifers. A partial RMV cDNA containing the 30K subgenomic promoter, 30K ORF, coat subgenomic promoter, coat ORF, and 3′-end was isolated by RT-PCR using by using oligonucleotides TVCV183X, 5′-TAC TCG AGG TTC ATA AGA CCG CGG TAG GCG G-3′ (upstream) (SEQ ID NO: 7) and TVCV KpnI, 5′-CGG GGT ACC TGG GCC CCT ACC CGG GGT TTA GGG AGG-3′ (downstream) (SEQ ID NO: 8), and subcloned into the EcoRV site of KS+, creating plasmid KS+ TVCV #3 (FIG. 3). The RMV cDNA was characterized by restriction mapping and dideoxy nucleotide sequencing. The partial nucleotide sequence is as follows:

| | |
|---|---|
| 5′-CTCGAGGTTCATAAGACCGCGGTAGGCGGAGCGT | (SEQ ID NO:9) |
| TTGTTTACTGTAGTATAATTAAATATTTGTCAGATAA | |
| AAGGTTGTTTAAAGATTTGTTTTTTGTTTGACTGAGT | |
| CGATAATGTCTTACGAGCCTAAAGTTAGTGACTTCCT | |
| TGCTCTTACGAAAAAGGAGGAAATTTTACCCAAGGCT | |
| TTGACGAGATTAAAGACTGTCTCTATTAGTACTAAGG | |
| ATGTTATATCTGTTAAGGAGTCTGAGTCCCTGTGTGA | |
| TATTGATTTGTTAGTGAATGTGCCATTAGATAAGTAT | |
| AGGTATGTGGGTGTTTTGGGTGTTGTTTTCACCGGTG | |
| AATGGCTGGTACCGGATTTCGTTAAAGGTGGGGTAAC | |
| AGTGAGCGTGATTGACAAACGGCTTGAAAATTCCAGA | |
| GAGTGCATAATTGGTACGTACCGAGCTGCTGTAAAGG | |
| ACAGAAGGTTCCAGTTCAAGCTGGTTCCAAATTACTT | |
| CGTATCCATTGCGGATGCCAAGCGAAAACCGTGGCAG | |
| GTTCATGTGCGAATTCAAAATCTGAAGATCGAAGCTG | |
| GATGGCAACCTCTAGCTCTAGAGGTGGTTTCTGTTGC | |
| CATGGTTACTAATAACGTGGTTGTTAAAGGTTTGAGG | |
| GAAAAGGTCATCGCAGTGAATGATCCGAACGTCGAAG | |
| GTTTCGAAGGTGTGGTTGACGATTTCGTCGATTCGGT | |
| TGCTGCATTCAAGGCGATTGACAGTTTCCGAAAGAAA | |
| AAGAAAAAGATTGGAGGAAGGGATGTAAATAATAATA | |
| AGTATAGATATAGACCGGAGAGATACGCCGGTCCTGA | |
| TTCGTTACAATATAAAGAAGAAAATGGTTTACAACAT | |
| CACGAGCTCGAATCAGTACCAGTATTTCGCAGCGATG | |
| TGGGCAGAGCCCACAGCGATGCTTAACCAGTGCGTGT | |
| CTGCGTTGTCGCAATCGTATCAAACTCAGGCGGCAAG | |
| AGATACTGTTAGACAGCAGTTCTCTAACCTTCTGAGT | |
| GCGATTGTGACACCGAACCAGCGGTTTCCAGAAACAG | |
| GATACCGGGTGTATATTAATTCAGCAGTTCTAAAACC | |
| GTTGTACGAGTCTCTCATGAAGTCCTTTGATACTAGA | |
| AATAGGATCATTGAAACTGAAGAAGAGTCGCGTCCAT | |
| CGGCTTCCGAAGTATCTAATGCAACACAACGTGTTGA | |
| TGATGCGACCGTGGCCATCAGGAGTCAAATTCAGCTT | |
| TTGCTGAACGAGCTCTCCAACGGACATGGTCTGATGA | |
| ACAGGGCAGAGTTCGAGGTTTTATTACCTTGGGCTAC | |
| TGCGCCAGCTACATAGGCGTGGTGCACACGATAGTGC | |
| ATAGTGTTTTTCTCTCCACTTAAATCGAAGAGATATA | |
| CTTACGGTGTAATTCCGCAAGGGTGGCGTAAACCAAA | |
| TTACGCAATGTTTTAGGTTCCATTTAAATCGAAACCT | |
| GTTATTTCCTGGATCACCTGTTAACGTACGCGTGGCG | |
| TATATTACAGTGGGAATAACTAAAAGTGAGAGGTTCG | |
| AATCCTCCCTAACCCCGGGTAGGGGCCCA-3′. | |

The 1543 base pair from the partial RMV cDNA was compared (PCGENE) to oilseed rape mosaic virus (ORMV). The nucleotide sequence identity was 97.8%. The RMV 30K and coat ORF were compared to ORMV and the amino acid identity was 98.11% (30K) and 98.73% (coat), respectively. A partial RMV cDNA containing the 5′-end and part of the replicase was isolated by RT-PCR from RMV RNA using oligonucleotides RGMV1 5′-GAT GGC GCC TTA ATA CGA CTC ACT ATA GTT TTA TTT TTG TTG CAA CAA CAA CAA C-3′ (upstream) (SEQ ID NO: 10) and RGR 132 5′-CTT GTG CCC TTC ATG ACG AGC TAT ATC ACG-3′ (downstream) (SEQ ID NO: 11). The RMV cDNA was characterized by dideoxy nucleotide sequencing. The partial nucleotide sequence containing the T7 RNA polymerase promoter and part of the RMV cDNA is as follows:

5′-ccttaatacgactcactataGTTTTATTTTTGTTGCAACAACAACAACAAATTACAATAA CAACAAAACAAATACAAACAACAACAACATGGCACAATTTCAACAAACAG TAAACATGCAAACATTGCAGGCTGCCGCAGGGCGCAACAGCCTGGTGAATG ATTTAGCCTCACGACGTGTTTATGACAATGCTGTCGAGGAGCTAAATGCACG CTCGAGACGCCCTAAGGTTCATTACTCCAAATCAGTGTCTACGGAACAGAC GCTGTTAGCTTCAAACGCTTATCCGGAGTTTGAGATTTCCTTTACTCATACCC AACATGCCGTACACTCCCTTGCGGGTGGCCTAAGGACTCTTGAGTTAGAGTA TCTCATGATGCAAGTTCCGTTCGGTTCTCTGACGTACGACATCGGTGGTAAC TTTGCAGCGCACCTTTTCAAAGGACGCGACTACGTTCACTGCTGTATGCCAA ACTTGGATGTACGTGATATAGCT-3′ (SEQ ID NO: 12). The uppercase letters are nucleotide sequences from RMV cDNA. The lower case letters are nucleotide sequences from T7 RNA polymerase promoter. The nucleotide sequences from the 5′and 3′ oligonucleotides are underlined.

Full length infectious RMV cDNA clones were obtained by RT-PCR from RMV RNA using oligonucleotides RGMV1, 5′-GAT GGC GCC TTA ATA CGA CTC ACT ATA GTT TTA TTT TTG TTG CAA CAA CAA CAA C-3′ (upstream) (SEQ ID NO: 13) and RG1 APE, 5′-ATC GTT TAA ACT GGG CCC CTA CCC GGG GTT AGG GAG G-3′ (downstream) (SEQ ID NO: 14). The RMV cDNA was characterized by dideoxy nucleotide sequencing. The partial nucleotide sequence containing the T7 RNA polymerase promoter and part of the RMV cDNA is as follows:

5′-CCTTAATACGACTCACTATAGTTTTATTTTTGTTGCAACAACAACAACAAAT TACAATAACAACAAAACAAATACAAACAACAACAACATGGCACAATTTCAA CAAACAGTAAACATGCAAACATTCCAGGCTGCCGCAGGGCGCAACAGCCTG GTGAATGATTTAGCCTCACGACGTGTTTATGACAATGCTGTCGAGGAGCTAA ATGCACGCTCGAGACGCCCTAAGGTTCATTACTCCAAATCAGTGTCTACGGA ACAGACGCTGTTAGCTTCAAACGCTTATCCGGAGTTTGAGATTTCCTTTACT CATACCCAAACATGCCGTACACTCCCTTGCGGGTGGCCTAAGGACTCTTGAG TTAGAGTATCTCATGATGCAAGTTCCGTTCGGTTCTCTGACGTACGACATCG GTGGTAACTTTGCAGCGCACCTTTTCAAAGGACGCGACTACGTTCACTGCTG TATGCCAAACTTGGATGTACGTGATATAGCT-3′ (SEQ ID NO: 15). The uppercase letters are nucleotide sequences from RMV cDNA. The nucleotide sequences from the 5′ and 3′ oligonucleotides are underlined. Full length infectious RMV cDNA clones were obtained by RT-PCR from RMV RNA using oligonucleotides RGMV1, 5′-gat ggc gcc tta ata cga ctc act ata gtt tta ttt ttg ttg caa caa caa caa c-3′ (upstream) (SEQ ID NO: 16) and RG1 APE, 5′-ATC GTT TAA ACT GGG CCC CTA CCC GGG GTT AGG GAG G-3′ (downstream) (SEQ ID NO: 17).

### Example 6

Arabidopsis thaliana cDNA librarm construction in a dual subgenomic promoter vector.

Arabidopsis thaliana cDNA libraries obtained from the Arabidopsis Biological Resource Center (ABRC). The four libraries from ABRC were size-fractionated with inserts of 0.5-1 kb (CD4-13), 1-2 kb (CD4-14), 2-3 kb (CD4-15), and 3-6 kb (CD4-16). All libraries are of high quality and have been used by several dozen groups to isolate genes. The pBluescript® phagemids from the Lambda ZAP II vector were subjected to mass excision and the libraries were recovered as plasmids according to standard procedures.

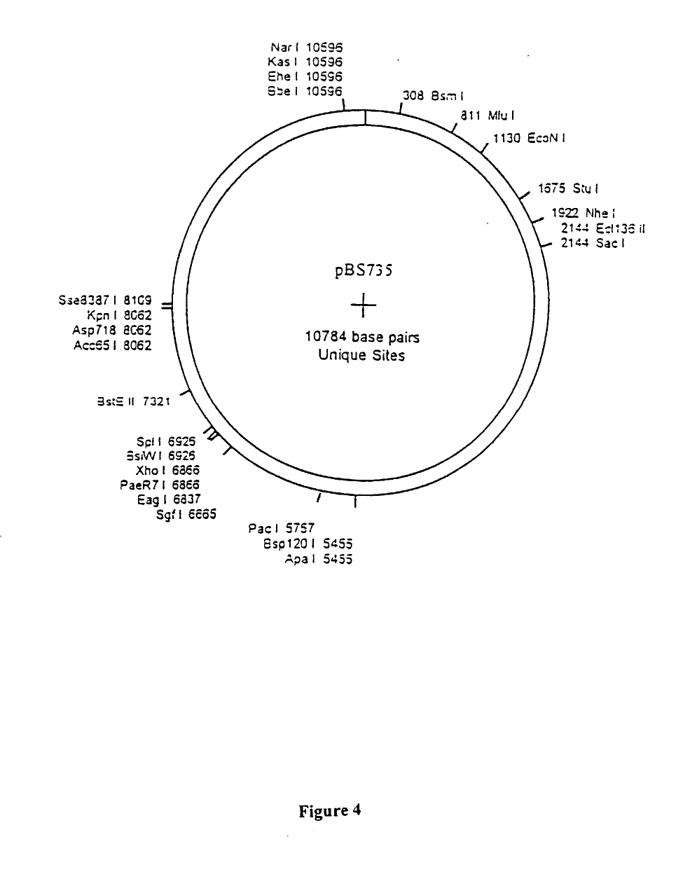

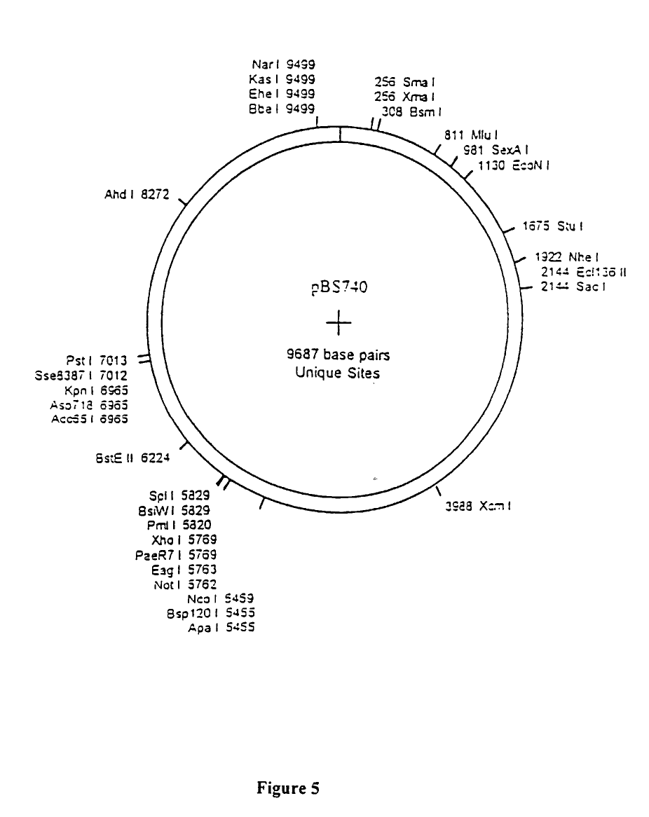

Alternatively, the cDNA inserts in the CD4-13 (Lambda ZAP II vector) were recovered by digestion with NotI. Digestion with NotI in most cases liberated the entire Arabidopsis thaliana cDNA insert because the original library was assembled with NotI adapters. NotI is an 8-base cutter that infrequently cleaves plant DNA. In order to insert the NotI fragments into a transcription plasmid, the pBS735 transcription plasmid (FIG. 4) was digested with PacI/XhoI and ligated to an adapter DNA sequence created from the oligonucleotides 5′-TCGAGCGGCCGCAT-3′ (SEQ ID NO: 18) and 5′-GCGGCCGC-3′ (SEQ ID NO: 19). The resulting plasmid pBS740 (FIG. 5) contains a unique NotI restriction site for bidirectional insertion of NotI fragments from the CD4-13 library. Recovered colonies were prepared from these for plasmid minipreps with a Qiagen BioRobot 9600®. The plasmid DNA preps performed on the BioRobot 9600® are done in 96-well format and yield transcription quality DNA. An Arabidopsis cDNA library was transformed into the plasmid and analyzed by agarose gel electrophoresis to identify clones with inserts. Clones with inserts are transcribed in vitro and inoculated onto N. benthamiana or Arabidopsis thaliana. Selected leaf disks from transfected plants are then taken for biochemical analysis.

### Example 7

High throughput robotics.

The efficiency of inoculation of subject organisms such as plants is improved by using means of high throughput robotics. For example, host plants such as Arabidopsis thaliana were grown in microtiter plates such as the standard 96-well and 384-well microtiter plates. A robotic handling arm then moved the plates containing the organism to a colony picker or other robot that delivered inoculations to each plant in the well. By this procedure, inoculation was performed in a very high speed and high throughput manner. It is preferable that the plant is a germinating seed or at least in the development cycle to enable access to the cells to be transfected. Equipments used for automated robotic production line include, but not be limited to, robots of these types: electronic multichannel pipetmen, Qiagen BioRobot 9600®, Robbins Hydra liquid handler, Flexys Colony Picker, New Brunswick automated plate pourer, GeneMachines HiGro shaker incubator, New Brunswick floor shaker, three Qiagen BioRobots, MJ Research PCR machines (PTC-200, Tetrad), ABI 377 sequencer and Tecan Genesis RSP200 liquid handler.

### Example 8

Genomic DNA library construction in a recombinant viral nucleic acid vector.

Genomic DNAs represented in BAC (bacterial artificial chromosome) or YAC (yeast artificial chromosome) libraries are obtained from the Arabidopsis Biological Resource Center (ABRC). The BAC/YAC DNAs are mechanically size-fractionated, ligated to adapters with cohesive ends, and shotgun-cloned into recombinant viral nucleic acid vectors. Alternatively, mechanically size-fractionated genomic DNAs are blunt-end ligated into a recombinant viral nucleic acid vector. Recovered colonies are prepared for plasmid minipreps with a Qiagen BioRobot 9600®. The plasmid DNA preps done on the BioRobot 9600® are assembled in 96-well format and yield transcription quality DNA. The recombinant viral nucleic acid/Arabidopsis genomic DNA library is analyzed by agarose gel electrophoresis (template quality control step) to identify clones with inserts. Clones with inserts are then transcribed in vitro and inoculated onto N. benthamiana and/or Arabidopsis thaliana. Selected leaf disks from transfected plants are then be taken for biochemical analysis.

Genomic DNA from Arabidopsis typically contains a gene every 2.5 kb (kilobases) on average. Genomic DNA fragments of 0.5 to 2.5 kb obtained by random shearing of DNA were shotgun assembled in a recombinant viral nucleic acid expression/knockout vector library. Given a genome size of Arabidopsis of approximately 120,000 kb, a random recombinant viral nucleic acid genomic DNA library would need to contain minimally 48,000 independent inserts of 2.5 kb in size to achieve 1× coverage of the Arabidopsis genome. Alternatively, a random recombinant viral nucleic acid genomic DNA library would need to contain minimally 240,000 independent inserts of 0.5 kb in size to achieve 1× coverage of the Arabidopsis genome. Assembling recombinant viral nucleic acid expression/knockout vector libraries from genomic DNA rather than cDNA has the potential to overcome known difficulties encountered when attempting to clone rare, low-abundance mRNA's in a cDNA library. A recombinant viral nucleic acid expression/knockout vector library made with genomic DNA would be especially useful as a gene silencing knockout library. In addition, the Dual Heterologous Subgenomic Promoter Expression System (DHSPES) expression/knockout vector library made with genomic DNA would be especially useful for expression of genes lacking introns. Furthermore, other plant species with moderate to small genomes (e.g. rose, approximately 80,000 kb) would be especially useful for recombinant viral nucleic acid expression/knockout vector libraries made with genomic DNA. A recombinant viral nucleic acid expression/knockout vector library can be made from existing BAC/YAC genomic DNA or from newly-prepared genomic DNAs for any plant species.

### Example 9

Genomic DNA or cDNA library construction in a DHSPES vector, and transfection of individual clones from said vector library onto T-DNA tagged or transposon tagged or mutated plants.

Genomic DNA or cDNA library construction in a recombinant viral nucleic acid vector, and transfection of individual clones from the vector library onto T-DNA tagged or transposon tagged or mutated plants may be performed according to the procedure set forth in Example 7. Such a protocol may be easily designed to complement mutations introduced by random insertional mutagenesis of T-DNA sequences or transposon sequences.

### Example 10

Identification of nucleotide sequences involved in the regulation of plant growth by cytoplasmic inhibition of gene expression using viral derived RNA (GTP binding proteins).

In the following examples, we show: (1) a method for producing antisense RNA using an RNA viral vector, (2) a method to produce viral-derived antisense RNA in the cytoplasm, (3) a method to inhibit the expression of endogenous plant proteins in the cytoplasm by viral antisense RNA, and (4) a method to produce transfected plants containing viral antisense RNA, such method is much faster than the time required to obtain genetically engineered antisense transgenic plants. Systemic infection and expression of viral antisense RNA occurs as short as four days post inoculation, whereas it takes several months or longer to create a single transgenic plant. These examples demonstrates that novel positive strand viral vectors, which replicate solely in the cytoplasm, can be used to identify genes involved in the regulation of plant growth by inhibiting the expression of specific endogenous genes. These examples enable one to characterize specific genes and biochemical pathways in transfected plants using an RNA viral vector.

Tobamoviral vectors have been developed for the heterologous expression of uncharacterized nucleotide sequences in transfected plants. A partial Arabidopsis thaliana cDNA library was placed under the transcriptional control of a tobamovirus subgenomic promoter in a RNA viral vector. Colonies from transformed E. coli were automatically picked using a Flexys robot and transferred to a 96 well flat bottom block containing terrific broth (TB) Amp 50 ug/ml. Approximately 2000 plasmid DNAs were isolated from overnight cultures using a BioRobot and infectious RNAs from 430 independent clones were directly applied to plants. One to two weeks after inoculation, transfected Nicotiana benthamiana plants were visually monitored for changes in growth rates, morphology, and color. One set of plants transfected with 740 AT #120 were severely stunted. DNA sequence analysis revealed that this clone contained an Arabidopsis GTP binding protein open reading frame (ORF) in the antisense orientation. This demonstrates that an episomal RNA viral vector can be used to deliberately alter the metabolic pathway and cause plant stunting. In addition, our results suggest that the Arabidopsis antisense transcript can turn off the expression of the N. benthamiana gene.

Construction of an Arabidopsis thaliana cDNA library in an RNA viral vector.

An Arabidopsis thaliana CD4-13 cDNA library was digested with NotI. DNA fragments between 500 and 1000 bp were isolated by trough elution and subcloned into the NotI site of pBS740. E. coli C600 competent cells were transformed with the pBS740 AT library and colonies containing Arabidopsis cDNA sequences were selected on LB Amp 50 ug/ml. Recombinant C600 cells were automatically picked using a Flexys robot and then transferred to a 96 well flat bottom block containing terrific-broth (TB) Amp 50 ug/ml. Approximately 2000 plasmid DNAs were isolated from overnight cultures using a BioRobot (Qiagen) and infectious RNAs from 430 independent clones were directly applied to plants.

Isolation of a gene encoding a GTP binding protein.

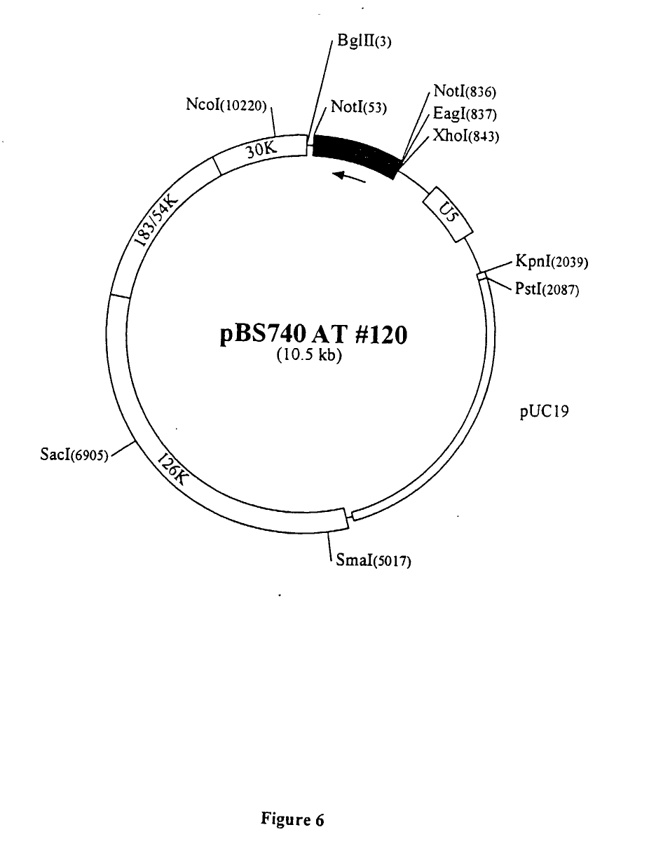

One to two weeks after inoculation, transfected Nicotiana benthamiana plants were visually monitored for changes in growth rates, morphology, and color. Plants transfected with 740 AT #120 (FIG. 6) were severely stunted. Plasmid 740 AT #120 contains the TMV-U1 126-, 183-, and 30-kDa ORFs, the TMV-U5 coat protein gene (U5 cp), the T7 promoter, an Arabidopsis thaliana CD4-13 NotI fragment, and part of the pUC19 plasmid. The TMV-U1 subgenomic promoter located within the minus strand of the 30-kDa ORF controls the synthesis of the CD4-13 antisense subgenomic RNA.

DNA sequencing and computer analysis.

A 782 bp NotI fragment of 740 AT #120 containing the ADP-ribosylation factor (ARF) cDNA was characterized. DNA sequence of NotI fragment of 740 AT #120 (774 base pairs) is as follows: 5′-

| | |
|---|---|
| 5′-CCGAAACATTCTTCGTAGTGAAGCAAAATGGGG | (SEQ ID NO:20) |
| TTGAGTTTCGCCAAGCTGTTTAGCAGGCTTTTTGCC | |
| AAGAAGGAGATGCGAATTCTGATGGTTGGTCTTGAT | |
| GCTGCTGGTAAGACCACAATCTTGTACAAGCTCAAG | |
| CTCGGAGAGATTGTCACCACCATCCCTACTATTGGT | |
| TTCAATGTGGAAACTGTGGAATACAAGAACATTAGT | |
| TTCACCGTGTGGGATGTCGGGGGTCAGGACAAGATC | |
| CGTCCCTTGTGAGACACTACTTCCAGAACACTCAAG | |
| GTCTAATCTTTGTTGTTGATAGCAATGACAGAGACA | |
| GAGTTGTTGAGGCTCGAGATGAACTCCACAGGATGC | |
| TGAATGAGGACGAGCTGCGTGATGCTGTGTTGCTTG | |
| TGTTTGCCAACAAGCAAGATCTTCCAAATGCTATGA | |
| ACGCTGCTGAAATCACAGATAAGCTTGGCCTTCACT | |
| CCCTCCGTCAGCGTCATTGGTATATCCAGAGCACAT | |
| GTGCCACTTCAGGTGAAGGGCTTTATGAAGGTCTGG | |
| ACTGGCTCTCCAACAACATCGCTGGCAAGGCATGAT | |
| GAGGGAGAAATTGCGTTGCATCGAGATGATTCTGTC | |
| TGCTGTGTTGGGATCTCTCTCTGTCTTGATGCAAGA | |
| GAGATTATAAATATTATCTGAACCTTTTTGCTTTTT | |
| TGGGTATGTGAATGTTTCTTATTGTGCAAGTAGATG | |
| GTCTTGTACCTAAAAATTTACTAGAAGAACCCTTTT | |
| AAATAGCTTTCGTGTATTGT-3′. | |

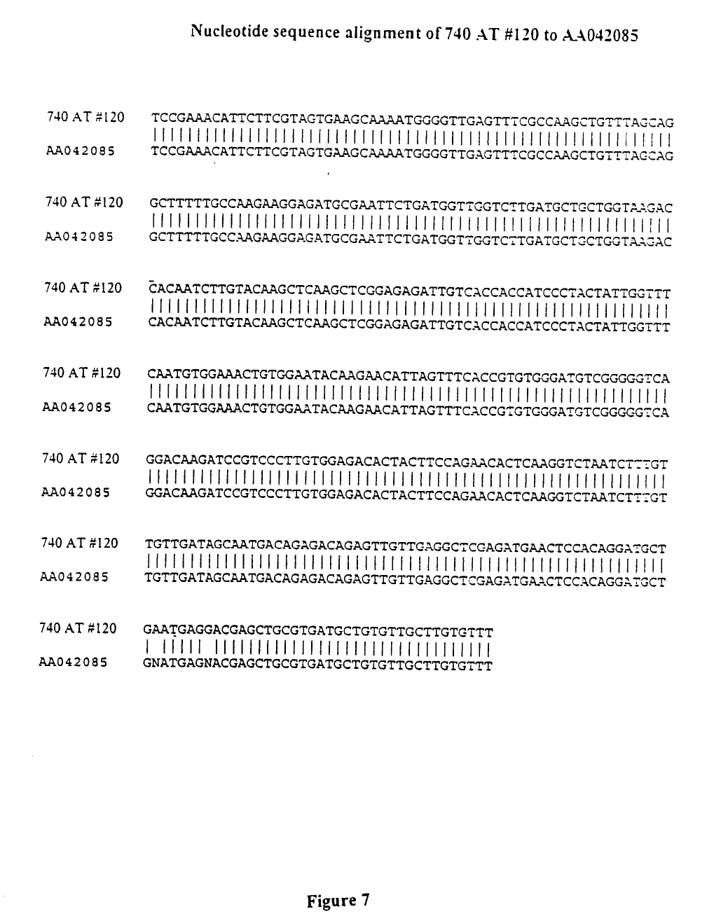

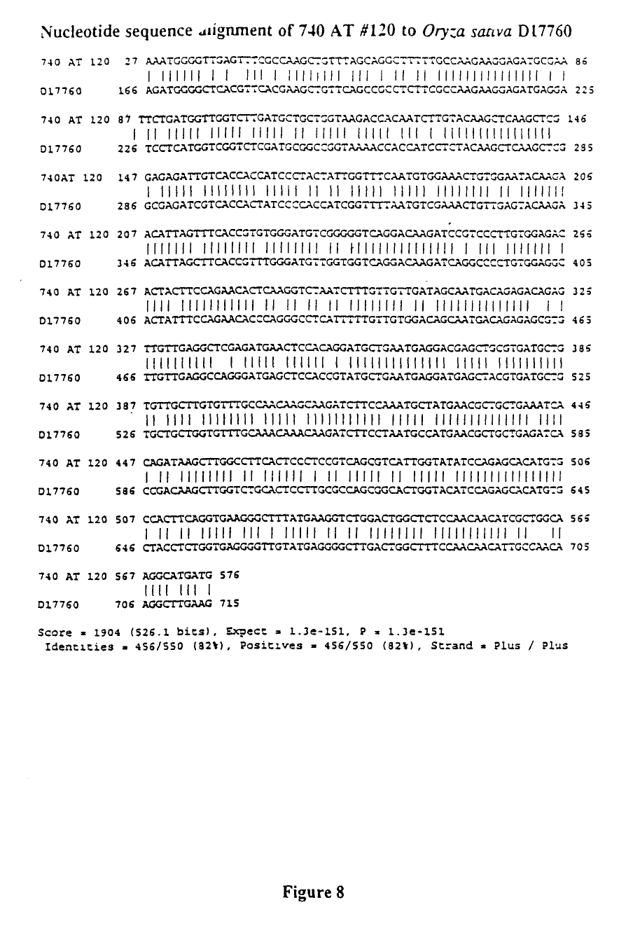

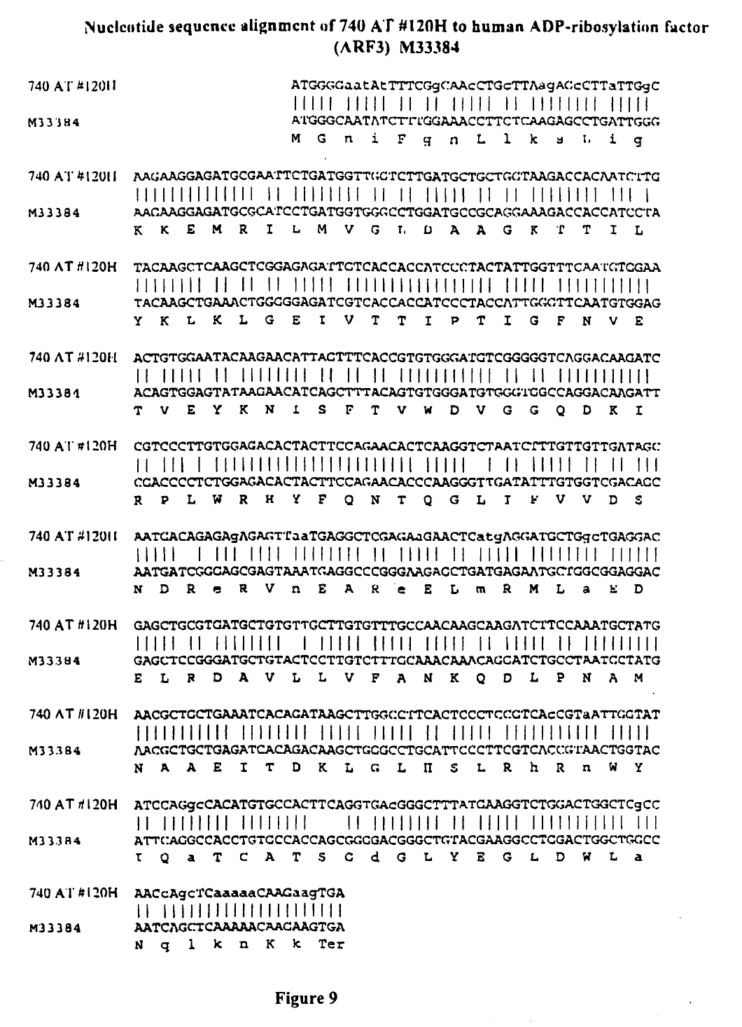

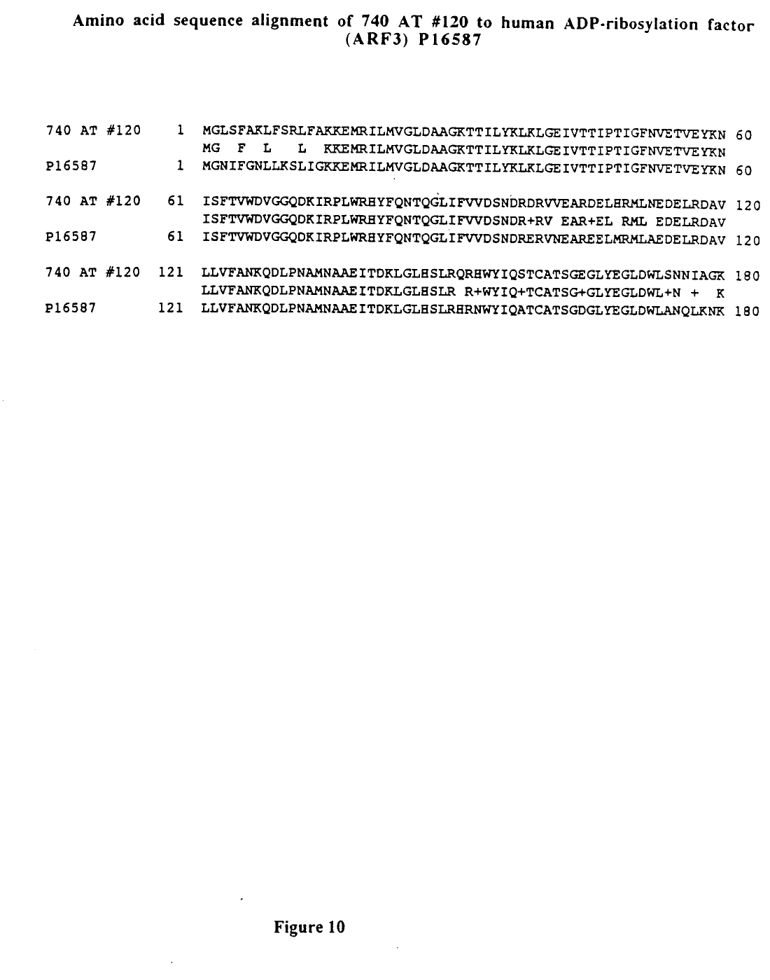

The nucleotide sequencing of 740 AT #120 was carried out by dideoxy termination using double stranded templates (Sanger et al. 1977). Nucleotide sequence analysis and amino acid sequence comparisons were performed using DNA Strider, PCGENE and NCBI Blast programs. 740 AT #120 contained an open reading frame (ORF) in the antisense orientation that encodes a protein of 181 amino acids with an apparent molecular weight of 20,579 Daltons. FIG. 7 shows the nucleotide sequence comparison of A. thalana 740 AT #120 and A. thaliana est AA042085. FIG. 8 shows the nucleotide sequence alignment of 740 AT #120 to rice Oryza sativa D17760 (82% identities and positives). The nucleotide sequence from 740 AT #120 is also compared with a human ADP ribosylation factor (ARF3) M33384, (FIG. 9), which shows a strong similarity (76% identity at the nucleotide level and 87% identity at the amino acid level). The amino acid sequence alignment of 740 AT #120 to human ADP-ribosylation factor (ARF3) P16587 is compared in FIG. 10, which shows 87% identity and 90% positive.

Humanizing DNA

The nucleotide sequence from 740 AT #120 is also compared with a human ADP ribosylation factor (ARF3) M33384 (FIG. 9), which shows a strong similarity (76% identity at the nucleotide level and 87% identity at the amino acid level). The high homology of the nucleic acid and amino acid sequence between the two makes humanizing 740 #AT120 practical. FIG. 9 shows the 740 AT#120 H nucleic acid sequence. The 740 AT#120 H nucleic acid sequence is prepared by changing the 740 AT#120 nucleic acid sequence so that it encodes the same amino acid sequence as the human M33384 encodes. The nucleic acid is changed by a standard method such as site directed mutagenisis or DNA synthesis.

The amino acid sequence alignment of 740 AT #120 to human ADP-ribosylation factor (ARF3) P16587 is again compared in FIG. 10, which shows 87% identity and 90% positive.

Comparison of nucleotide sequences from different organisms

The nucleotide sequence from 740 AT #120 exhibits a high degree of homology (71-84% identity and positive) to DNA encoding ARFs from yeast, plants, insects such as fly, amphibian such as frog, mammalian such as bovine, human, and mouse DNA encoding ARFs (Table 1). The amino acid sequence derived from 740 AT #120 exhibits an even higher degree of homology (61-98% identity and 78-98% positive) to ARFs from yeast, plants insects such as fly, mammalian such as bovine, human, and mouse(Table 2). The high homology of DNAs encoding GTP binding proteins from yeast, plants, insects, human, mice, and amphibians indicates that DNAs from one donor organism can be transfected into another host organism and silence the endogenous gene of the host organism.

The protein encoded by 740 AT #120, 120P, contained three conserved domains: the phosphate binding loop motif, GLDAAGKT (consensus GXXXXGKS/T); the G′ motif, DVGGQ , (consensus DXXGQ), a sequence which interacts with the gamma-phosphate of GTP; and the G motif NKQD, (consensus NKXD), which is specific for guanidinyl binding. The 120P contains a putative glycine-myristoylation site at position 2, a potential N-glycosylation site (NXS) at position 60, and several putative serine/threonine phosphorylations sites.

TABLE 1
740 AT #120 Nucleotide sequence comparison

| | Score | Expect | Identities | Positive |
|---|---|---|---|---|
| barley E10542 | 540.8 bits (1957) | 1.4e−157 | 461/548 (84%) | 461/548 (84%) |
| A. thaliana M95166 | 538.5 bits (1949) | 7.4e−157 | 461/550 (83%) | 461/550 (83%) |
| rice AF012896 | 537.7 bits (1946) | 1.3e−156 | 462/553 (83%) | 462/553 (83%) |
| carrot D45420 | 531.4 bits (1923) | 9.8e−155 | 471/579 (81%) | 471/579 (81%) |
| corn X80042 | 512.3 bits (1854) | 6.8e−149 | 450/549 (81%) | 450/549 (81%) |
| C. reinhardtii U27120 | 480.0 bits (1740) | 1.6e−139 | 436/546 (79%) | 436/546 (79%) |
| mouse brain ARF3 D87900 | 431.1 bits (1560) | 1.7e−124 | 416/546 (76%) | 416/546 (76%) |
| Bovine J03794 | 426.9 bits (1545) | 3.6e−123 | 409/534 (76%) | 409/534 (76%) |
| Human ARF3 M33384 | 433.5 bits (1569) | 4.9e−123 | 417/546 (76%) | 417/546 (76%) |
| S. pombe ARF1 L09551 | 430.2 bits (1557) | 1.1e−121 | 409/531 (77%) | 409/531 (77%) |
| Human ARF1 AF05502 | 428 bits (1549) | 5.8e−121 | 405/524 (77%) | 405/524 (77%) |
| frog U31350 | 414.5 bits (1500) | 1.7e−119 | 412/552 (74%) | 412/552 (74%) |
| Human ARF5 M57567 | 387.4 bits (1402) | 1.0e−107 | 390/527 (74%) | 390/527 (74%) |
| S. cerevisiae J03276 | 362.8 bits (1313) | 1.6e−99 | 381/529 (72%) | 381/529 (72%) |
| Human ARF4 M36341 | 358.4 bits (1297) | 4.3e−98 | 377/524 (71%) | 377/524 (71%) |
| C. elegans M36341 | 149.8 bits (542) | 2.0e−90 | 154/211 (72%) | 154/211 (72%) |
| N. tabacum NTGB1 U46927 | 285.7 bits (1034) | 4.8e−78 | 234/268 (87%) | 234/268 (87%) |
| Human cosmid AC000357 | 107.5 bits (389) | 9.7e−73 | 93/112 (83%) | 93/112 (83%) |
| fly S62079 | 211.9 bits (767) | 2.8e−72 | 195/247 (78%) | 195/247 (78%) |

TABLE 2
Amino acid sequence comparison of 740
AT #120 with ARFs from other organisms.

| | Score | Expect | Identities | Positives |
|---|---|---|---|---|
| A. thaliana | 365 bits | e−101 | 179/181 (98%) | 179/181 (98%) |
| ARF1 g543841 | (928) | | | |
| rice g1703380 | 363 bits | e−100 | 177/181 (97%) | 179/181 (98%) |
| | (921) | | | |
| corn g1351974 | 356 bits | 3e−98 | 174/181 (96%) | 179/181 (98%) |
| | (905) | | | |
| carrot | 362 bits | e−100 | 177/181 (97%) | 178/181 (97%) |
| g1703375 | (919) | | | |
| C. reinhardtii | 354 bits | 2e−97 | 172/180 (95%) | 174/180 (96%) |
| g1703374 | (898) | | | |
| Bovine | 327 bits | 2e−89 | 160/177 (90%) | 160/177 (93%) |
| | (829) | | | |
| Human ARF1 | 326 bits | 4e−89 | 160/177 (90%) | 160/177 (93%) |
| | (827) | | | |
| mouse | 326 bits | 4e−89 | 160/177 (90%) | 160/177 (93%) |
| | (827) | | | |
| fly | 325 bits | 7e−89 | 158/177 (89%) | 160/177 (93%) |
| | (825) | | | |
| Human ARF3 | 321 bits | 1e−87 | 157/180 (87%) | 164/180 (90%) |
| P16587 | (813) | | | |
| Human ARF3 | 305 bits | 7e−83 | 145/178 (81%) | 161/178 (89%) |
| g114127 | (774) | | | |
| Human ARF4 | 304 bits | 2e−82 | 145/178 (81%) | 160/178 (89%) |
| g114123 | (770) | | | |
| yeast ARF1 | 298 bits | 2e−80 | 139/177 (78%) | 161/177 (90%) |
| g171072 | (754) | | | |
| A. thaliana | 241 bits | 2e−63 | 109/177 (61%) | 140/177 (78%) |
| ARF3 | (608) | | | |

### Example 11

Isolation of an Arabidopsis thaliana ARF genomic clone

A genomic clone encoding A. thaliana ARF can be isolated by probing filters containing A. thaliana BAC clones using a ³²p labelled 740 AT #120 NotI insert. Other members of the A. thaliana ARF multigene family have been identified using programs of the University of Wisconsin Genetic Computer Group. The BAC clone T08113 located on chromosome II has a high degree of homology to 740 AT #120 (78% to 86% identity) at the nucleotide level.

### Example 12

Construction of a Nicotiana benthamiana cDNA library.

Vegetative N. benthamiana plants were harvested 3.3 weeks after sowing and sliced up into three groups of tissue: leaves, stems and roots. Each group of tissue was flash frozen in liquid nitrogen and total RNA was isolated from each group separately using the following hot borate method (Larry Smart and Thea Wilkins, 1995). Frozen tissue was ground to a fine powder with a pre-chilled mortar and pestle, and then further homogenized in pre-chilled glass tissue grinder. Immediately thereafter, 2.5 ml/g tissue hot (˜82° C.) XT Buffer (0.2 M borate decahydrate, 30 mM EGTA, 1% (w/v) SDS. Adjusted pH to 9.0 with 5 N NaOH, treated with 0.1% DEPC and autoclaved. Before use, added 1% deoxycholate (sodium salt), 10 mM dithiothreitol, 15 Nonidet P-40 (NP-40) and 2% (w/v) polyvinylpyrrilidone, MW 40,000 (PVP-40)) was added to the ground tissue. The tissue was homogenized 1-2 minutes and quickly decanted to a pre-chilled Oak Ridge centrifuge tube containing 105 μl of 20 mg/ml proteinase K in DEPC treated water. The tissue grinder was rinsed with an additional 1 ml hot XT Buffer per g tissue, which was then added to rest of the homogenate. The homogenate was incubated at 42° C. at 100 rpm for 1.5 h. 2 M KCl was added to the homogenate to a final concentration of 160 mM, and the mixture was incubated on ice for 1 h to precipitate out proteins. The homogenate was centrifuged at 12,000×g for 20 min at 4° C., and the supernatant was filtered through sterile miracloth into a clean 50 ml Oak Ridge centrifuge tube. 8 M LiCl was added to a final concentration of 2 M LiCl and incubated on ice overnight. Precipitated RNA was collected by centrifugation at 12,000×g for 20 min at 4° C. The pellet was washed three times in 3-5 ml 4° C. 2 M LiCl. Each time the pellet was resuspended with a glass rod and then spun at 12,000×g for 20 min at 4° C. The RNA pellet was suspended in 2 ml 10 mM Tris-HCl (pH 7.5), and purified from insoluble cellular components by spinning at 12,000×g for 20 min at 4° C. The RNA containing supernatant was transferred to a 15 ml Corex tube and precipitated overnight at −20° C. with 2.5 volumes of 100 % ethanol. The RNA was pelleted by centrifugation at 9,800×g for 30 min at 4° C. The RNA pellet was washed in 1-2 ml cold 70° C ethanol and centrifuged at 9,800×g for 5 min at 4° C. Residual ethanol was removed from the RNA pellet under vacuum, and the RNA was resuspended in 200 μl DEPC treated dd-water and transferred to a 1.5 ml microfuge tube. The Corex tube was rinsed in 100 μl DEPC-treated dd-water, which was then added to the rest of the RNA. The RNA was then precipitated with 1/10 volume of 3 M sodium acetate, pH 6.0 and 2.5 volumes of cold 100% ethanol at −20° C. for 1-2 h. The tube was centrifuged for 20 min at 16,000×g, and the RNA pellet washed with cold 70% ethanol, and centrifuged for 5 min at 16,000×g. After drying the pellet under vacuum, the RNA was resuspended in DEPC-treated water. This is the total RNA.

Messenger RNA was purified from total RNA using an Poly(A)Pure kit (Ambion, Austin Tex.), following the manufacturer's instructions. A reverse transcription reaction was used to synthesize cDNA from the mRNA template, using either the Stratagene (La Jolla, Calif.) or Gibco BRL (Gaithersburg, Md.) cDNA cloning kits. For the Stratagene library, the cDNAs were subdloned into bacteriophage at EcoR1/XhoI site by ligating the arms using the Gigapack III Gold kit (Stratagene, La Jolla, Calif.), following the manufacturer's instructions. For the Gibco BRL library, the cDNAs were subcloned into a tobamoviral vector that contained a fusion of TMV-U1 and TMV-U5 at the NotI/XhoI sites.

### Example 13

Isolation and characterization of a cDNA encoding Nicotiana benthamiana ADP-ribosylation factor.

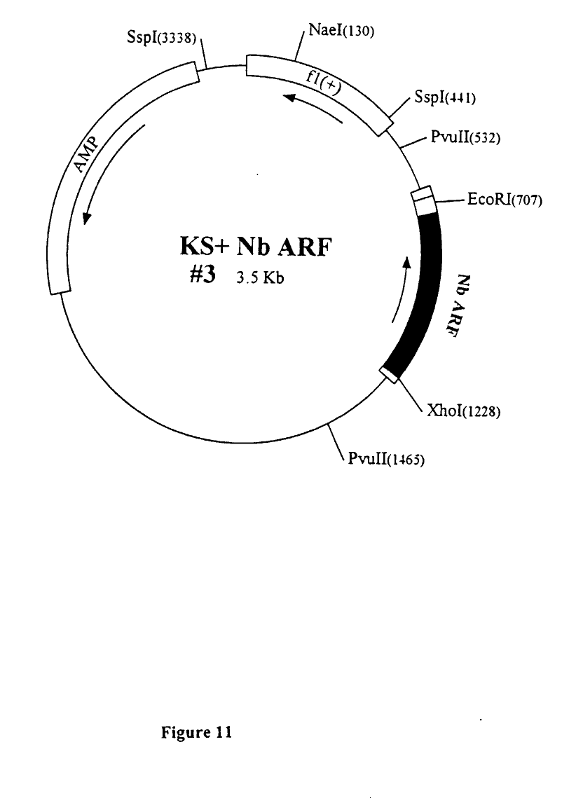

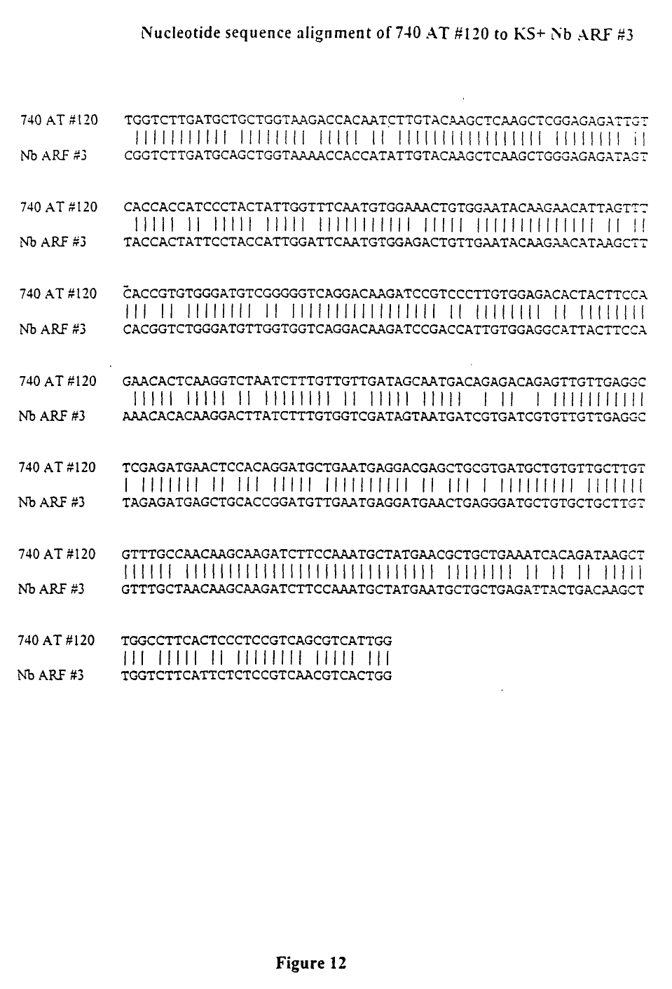

A 488 bp cDNA from N. benthamiana stem cDNA library was isolated by polymerase chain reaction (PCR) using the following oligonucleotides: ATARFK15, 5′ AAG AAG GAG ATG CGA ATT CTG ATG GT 3′ (upstream)(SEQ ID NO:42), ATARFN176, 5′ ATG TTG TTG GAG AGC CAG TCC AGA CC 3′ (downstream)(SEQ ID NO: 43). The vent polymerase in the reaction was inactivated using phenol/chloroform, and the PCR product was directly cloned into the HincII site in Bluescript KS+ (Strategene). The plasmid map of KS+ Nb ARF #3, which contains the N. benthamiaca ARF ORF in pBluescript KS+ is shown in FIG. 11. The nucleotide sequence of N. benthamiana KS+ Nb ARF#3, which contains partial ADP-ribosylation factor ORF, was determined by dideoxynucleotide sequencing. The nucleotide sequence from KS+ Nb ARF#3, had a strong similarity to other plant ADP-ribosylation factor sequences (82 to 87% identities at the nucleotide level). The nucleotide sequence comparison of N. benthamiana KS+ Nb AR-F#3 and A. thaliana 740 AT #120 shows a high homology between them (FIG. 12). The nucleotide sequence of KS+ NbARF #3 exhibits a high degree of homology (77-87% identities and positives) to plant, yeast and mammalian DNA encoding ARFs (Table 3). Again, the high homology of DNAs encoding GTP binding proteins from yeast, plants, human, bovine and mice indicates that DNAs from one donor organism can be transfected into another host organism and effectively silence the endogenous gene of the host organism.

A full-length cDNA encoding ARF is isolated by screening the N. benthamiana cDNA library by colony hybridization using a ³²P-labeled N. benthamiana KS+/Nb ARF #3 probe. Hybridization is carried out at 42° C. for 48 hours in 50% formamide, 5×SSC, 0.02 M phosphate buffer, 5×Denhart's solution, and 0.1 mg/ml sheared calf thymus DNA. Filters are washed at 65° C. in 0.1×SSC, 0.1% SDS prior to autoradiography.

TABLE 3
KS + Nb ARF #3 Nucleotide sequence comparison

| | Score | Expect | Identities | Positives |
|---|---|---|---|---|
| A thaliana M95166 | 448.2 bits (1622) | 1.2e−129 | 366/418 (87%) | 366/418 (87%) |
| C. roseus AF005238 | 446.0 bits (1614) | 5.3e−129 | 368/427 (86%) | 370/427 (86%) |
| S. bakko AB003377 | 444.9 bits (1610) | 1.1e−128 | 366/421 (86%) | 366/421 (86%) |
| rice AF012896 | 425.8 bits (1541) | 5.1e−121 | 357/418 (85%) | 357/418 (85%) |
| V. unguiculata AF022389 | 425.8 bits (1541) | 5.1e−121 | 857/418 (85%) | 357/418 (85%) |
| barley E10542 | 413.4 bits (1496) | 1.2e−115 | 356/427 (83%) | 356/427 (83%) |
| S. tuberosum X74461 | 405.9 bits (1469) | 3.5e−115 | 353/427 (82%) | 353/427 (82%) |
| carrot D45420 | 408.4.4 bits (1478) | 3.3e−114 | 354/427 (82%) | 354/427 (82%) |
| corn X80042 | 400.1 bits (1448) | 2.3e−113 | 348/421 (82%) | 348/421 (82%) |
| rice D17760 | 403.4 bits (1460) | 3.7e−112 | 352/427 (82%) | 352/427 (82%) |
| C reinhardtii U27120 | 373.6 bits (1352) | 5.0e−103 | 340/427 (79%) | 340/427 (79%) |
| Human ARF3 M33384 | 367.5.5 bits (1330) | 7.1e−101 | 334/419 (79%) | 334/419 (79%) |
| mouse brain ARF3 D87900 | 355.3 bits (1286) | 1.3e−97 | 330/421 (78%) | 330/421 (78%) |
| Bovine J03794 | 342.6 bits (1240) | 1.4e−95 | 324/419 (77%) | 324/419 (77%) |

### Example 14

Rapid isolation of cDNAs encoding human ADP-ribosylation factor

Libraries containing full-length human cDNAs from organisms such as brain, liver, breast, lung, etc. are obtained from public and private sources or prepared from human mRNAs. The cDNAs are inserted in viral vectors or in small subcloning vectors such as pBluescript (Strategene), pUC18, M13, or pBR322. Transformed bacteria (E. coli) are then plated on large petri plates or bioassay plates containing the appropriate media and antibiotic. Individual clones are selected using a robotic colony picker and arrayed into 96 well microtiter plates. The cultures are incubated at 37° C. until the transformed cells reach log phase. Aliquots are removed to prepare glycerol stocks for long term storage at −80° C. The remainder of the culture is used to inoculate an additional 96 well microtiter plate containing selective media and grown overnight. DNAs are isolated from the cultures and stored at −20° C. Using a robotic unit such as the Qbot (Genetix), the E. coli transformants or DNAs are rearrayed at high density on nylon or nitrocellulose filters or glass slides. Full-length cDNAs encoding ARFs from human brain, liver, breast, lung, etc. are isolated by screening the various filters or slides by hybridization with a ³²P-labeled or fluorescent N. benthamiana KS+/Nb ARF #3 probe, or ³²P-labeled Arabidopsis 740 AT #120 NotI insert.

### Example 15

Construction of a viral vector containing human cDNAs.

An ARF5 clone containing nucleic acid inserts from a human brain cDNA library (Bobak et al., Proc. NatL. Acad. Su. USA 86:6101-6105 (1989)) was isolated by polymerase chain reaction (PCR) using the following oligonucleotides: HARFMI, 5′ TAC CTA GGG CAA TAT CTT TGG AAA CCT TCT CAA G 3′ (upstream)(SEQ ID NO: 44), HARFK181X, 5′ CGC TCG AGT CAC TTC TTG TTT TTG AGC TGA TTG GCC AG 3′ (downstream)(SEQ ID NO: 45). The vent polymerase in the reaction was inactivated using phenol/chloroform. The PCR products are directly cloned into the XhoI, AvrII site TTO1A.

### Example 16

Identification of human nucleotide sequences involved in the regulation of plant growth by cytoplasmic inhibition of gene expression using viral derived RNA containing human nucleotide sequences.

A human brain cDNA library are obtained from public and private sources or prepared from human mRNAs. The cDNAs are inserted in viral rectors or in small subcloning vectors and the cDNA inserts are isolated from the cloning vectors with appropriate enzymes, modified, and NotI linkers are attached to the cDNA blunt ends. The human cDNA inserts are subcloned into the NotI site of pBS740. E. coli C600 competent cells are transformed with the pBS740 sublibrary and colonies containing human cDNA sequences are selected on LB Amp 50 ug/ml. DNAs containing the viral human brain cDNA library are purified from the transformed colonies and used to make infectious RNAs that are directly applied to plants. One to three weeks post transfection, the plants developing severe stunting phenotypes are identified and their corresponding viral vector inserts are characterized by nucleic acid sequencing.

Humanizing plant homolog for expression of plant derived human protein

In order to obtain the corresponding plant cDNAs, the human clones responsible for causing changes in the transfected plant phenotype (for example, stunting) are used as probes. Full-length plant cDNAs are isolated by hybridizing filters or slides containing N. benthamiana cDNAs with ³²P-labelled or fluorescent human cDNA insert probes. The positive plant clones are characterized by nucleic acid sequencing and compared with their human homologs. Plant codons that encode for different amino acids are changed by site directed mutagenesis to codons that encode for the same amino acids as their human homologs. The resulting “humanized” plant cDNAs encode an identical protein as the human clone. The “humanized” plant clones are used to produce human proteins in either transfected or transgenic plants by standard techniques. Because the “humanized” cDNA is from a plant origin, it is optimal for expression in plants.

### Example 17

Identification of Arabidopsis nucleotide sequences involved in the regulation of plant development and comparison with octopus rhodopsin cDNA.

This example again demonstrates that an episomal RNA viral vector can be used to deliberately manipulate a signal transduction pathway in plants, and identify nucleic acid sequences that involved the regulation of plant development.

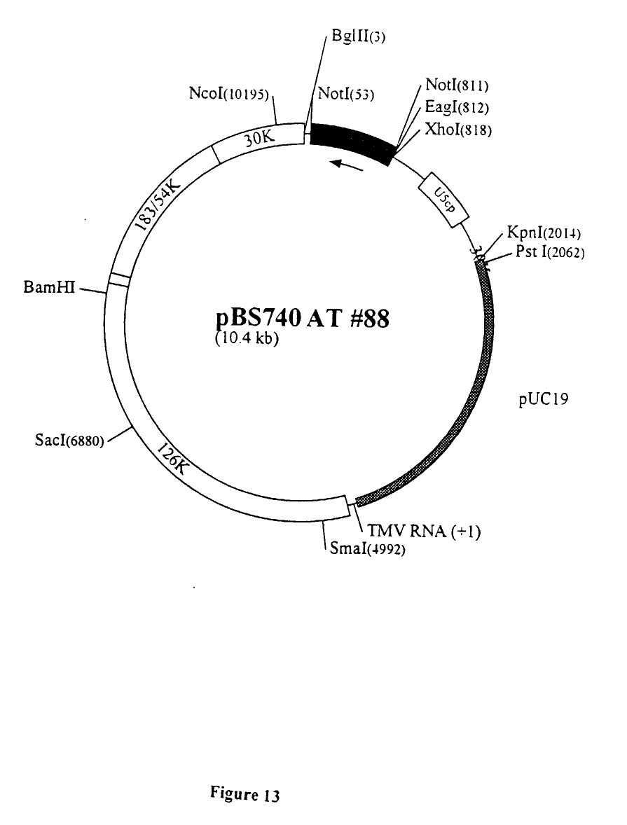

A partial Arabidopsis thaliana cDNA library was placed under the transcriptional control of a tobamovirus subgenomic promoter in a RNA viral vector. Colonies from transformed E. coli were automatically picked using a Flexys robot and transferred to a 96 well flat bottom block containing terrific broth (TB) Amp 50 ug/ml. Approximately 2000 plasmid DNAs were isolated from overnight cultures using a BioRobot and infectious RNAs from 430 independent clones were directly applied to plants. One to two weeks after inoculation, transfected Nicotiana benthamiana plants were visually monitored for changes in growth rates, morphology, and color. One set of plants transfected with 740 AT #88 (FIG. 13) developed a white phenotype on the infected leaf tissue. DNA sequence analysis revealed that this clone contained an Arabidopsis G-protein coupled receptor open reading frame (ORF) in the antisense orientation.

DNA sequencing and computer analysis.

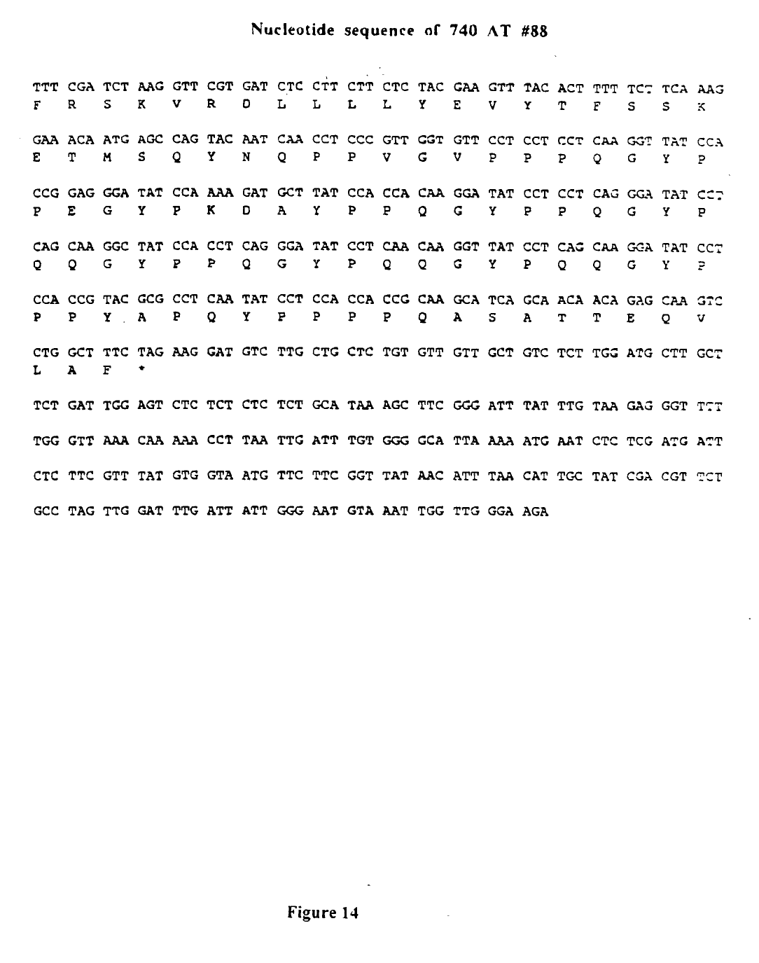

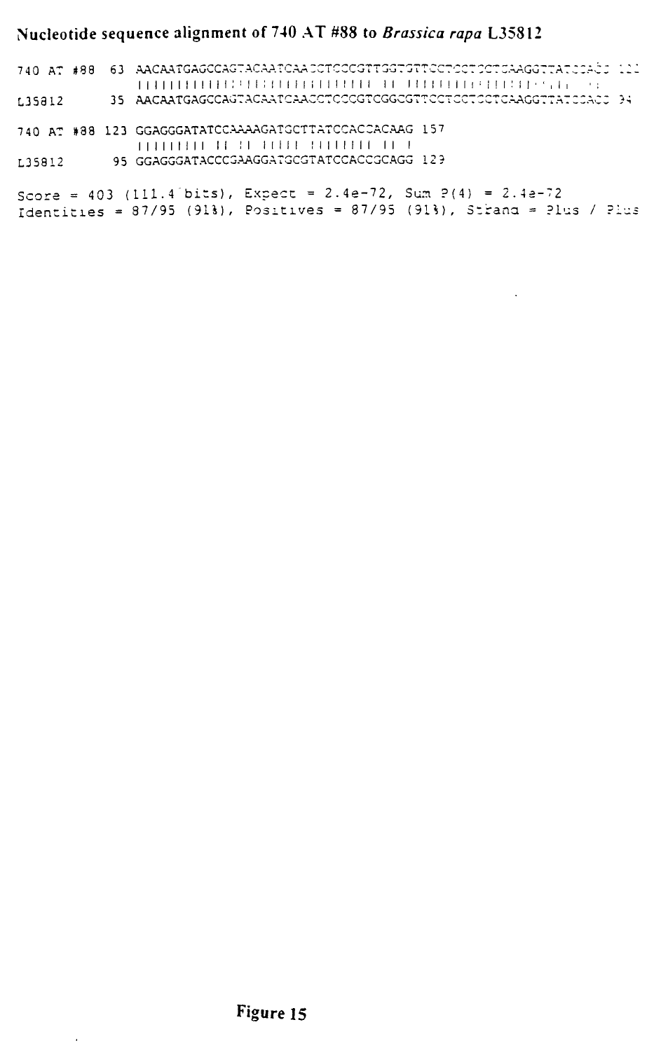

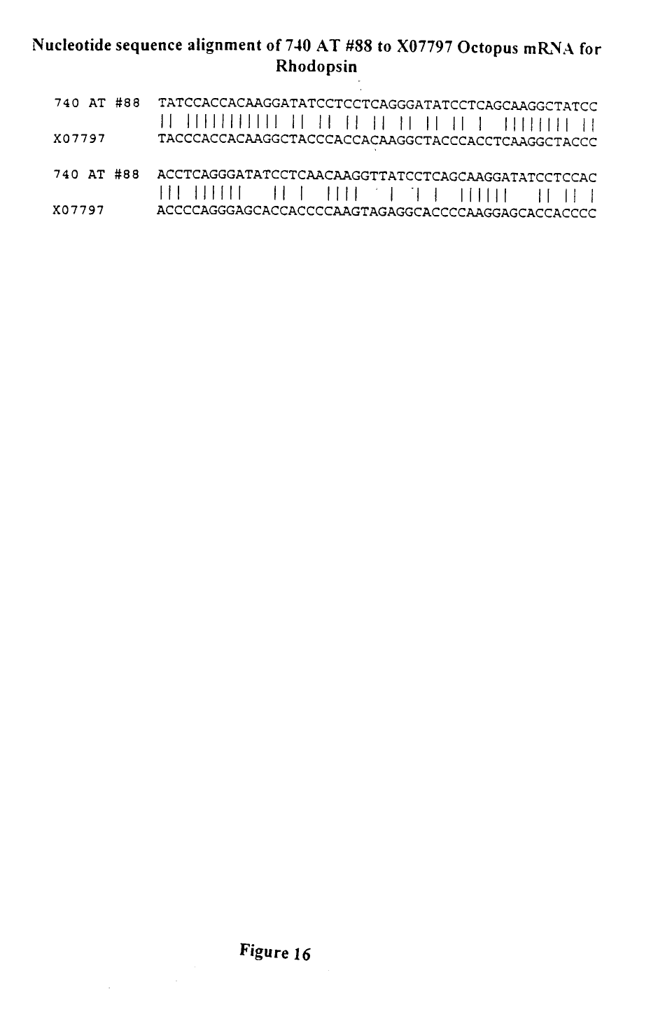

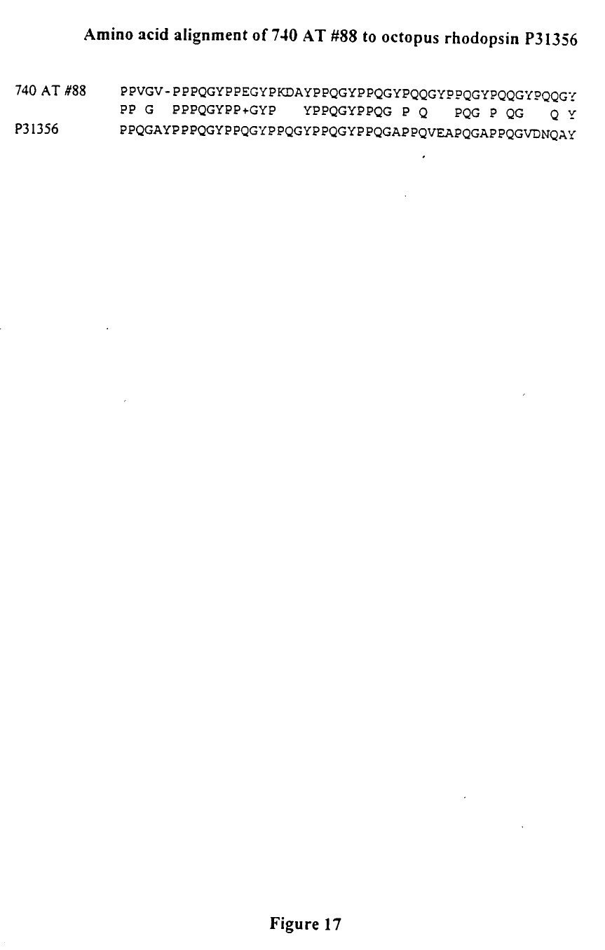

A 758 bp NotI fragment of 740 AT #88 containing the G-protein coupled receptor cDNA was characterized. The nucleotide sequencing of 740 AT #88 was carried out by dideoxy termination using double stranded templates (Sanger et al., 1977). Nucleotide sequence analysis and amino acid sequence comparisons were performed using DNA Strider, PCGENE and NCBI Blast programs. FIG. 14 shows the partial nucleotide sequence and amino acid sequence of 740 AT #88 insert. The nucleotide sequence from 740 AT #88 was compared with Brassica rapa cDNA L35812 (FIG. 15), 91% identities and positives; and the octopus rhodopsin cDNA X07797 (FIG. 16), 68% identities and positives. The homology of DNAs encoding rhodopsin from plant and animal rhodopsin indicates that genes from oen kingdom can inhibit the expression of gene of another kingdom. The amino acid sequence derived from 740 AT #88 was compared with octopus rhodopsin P31356 (FIG. 17), 65% identities and positives. Table 4 shows the amino acid sequence comparison of 740 AT #88 with D. discoideum and Octopus rhodopsin: 58-62% identities and 63-65% positives are shown.

### Example 18

Identification of nucleotide sequences containing an Arabidopsis S18 ribosomal protein open reading frame.

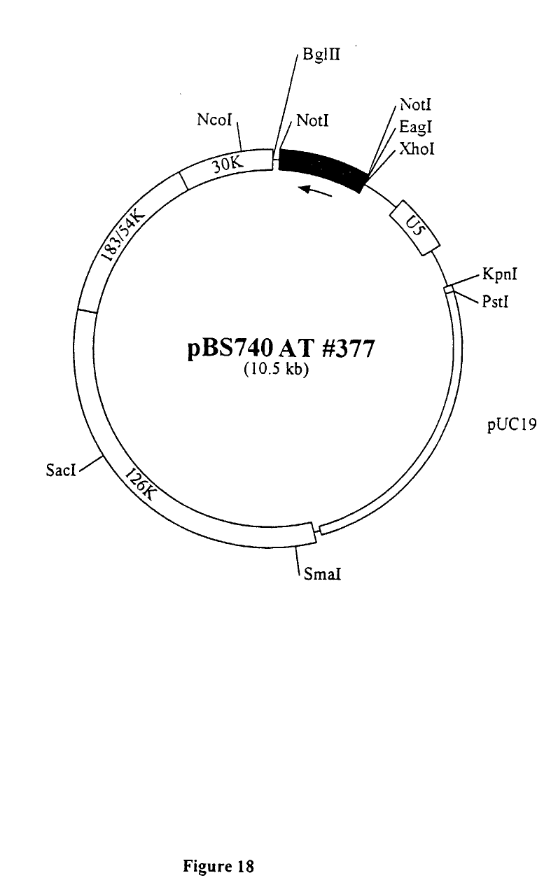

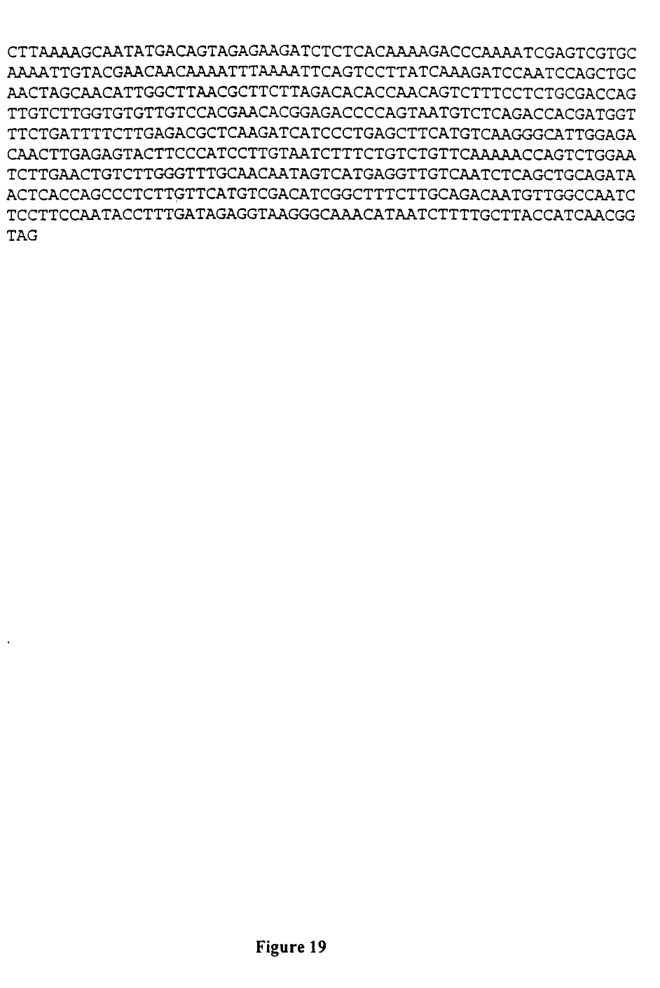

One to two weeks after inoculation, transfected Nicotiana benthamiana plants were visually monitored for changes in growth rates, morphology, and color. One set of plants transfected with 740 AT #377 (FIG. 18) were severely stunted. DNA sequence analysis (FIG. 19) revealed that this clone contained an Arabidopsis S18 ribosomal protein open reading frame (ORF) in the antisense orientation.

### Example 19

Identification of L19 ribosomal protein gene involved in the regulation of plant growth by cytoplasmic inhibition of gene expression using viral derived RNA.

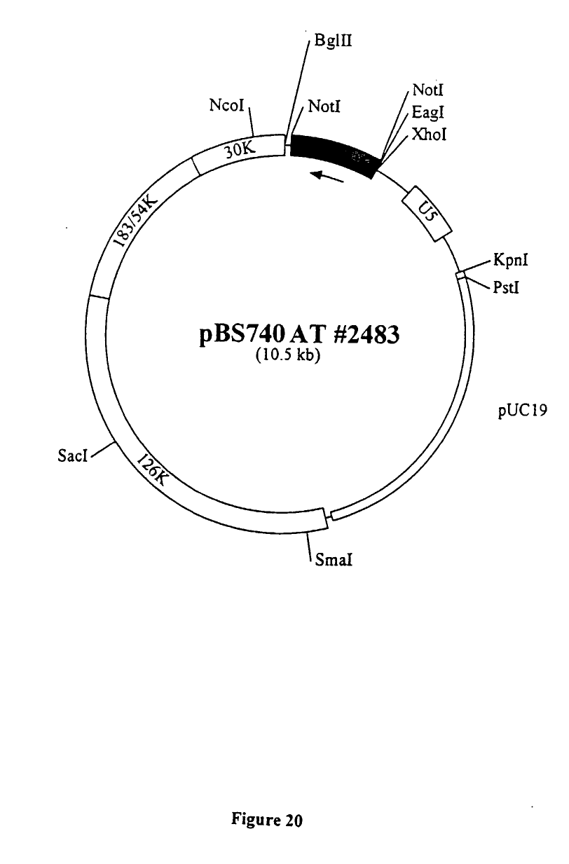

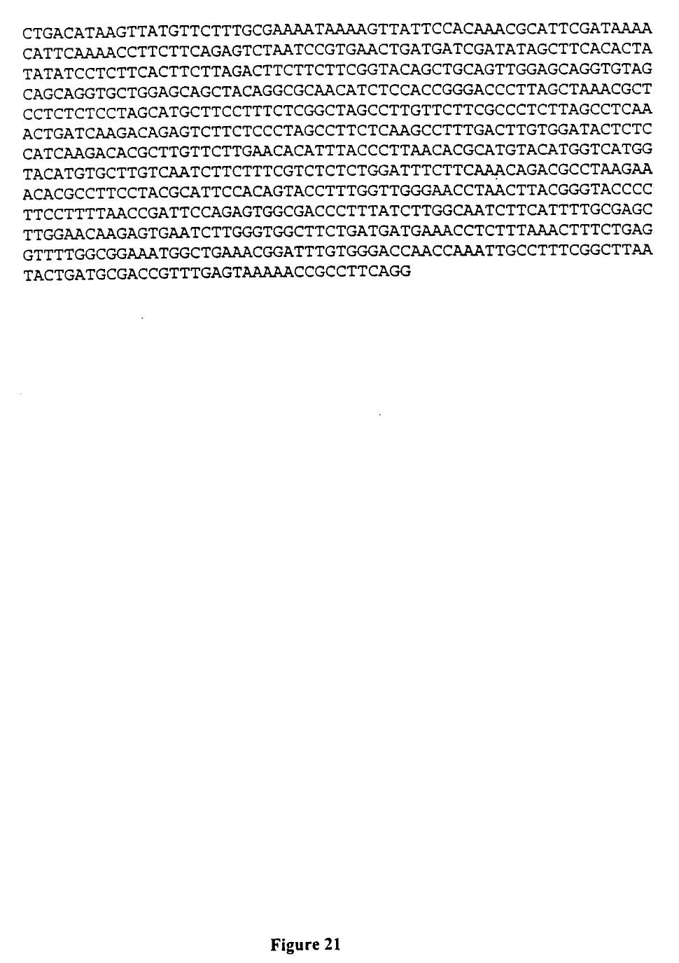

One to two weeks after inoculation, transfected Nicotiana benthamiana plants were visually monitored for changes in growth rates, morphology, and color. One set of plants transfected with 740 AT #2483 (FIG. 20) were severely stunted. DNA sequence analysis (FIG. 21) revealed that this clone contained an Arabidopsis L19 ribosomal protein open reading frame (ORF) in the antisense orientation. The 740 AT #2483 nucleotide sequence exhibited a high degree of homology (71-79% identities and positives) to plant, yeast, insect and human L19 ribosomal proteins genes (Table 5). The 740 AT #2483 amino acid sequence comparison with human, insect and yeast ribosomal protein L19 shows 38-88% identities and 61-88% positives (Table 6). The high homology of DNAs encoding ribosomal L19 protein from human, plant, yeast and insect indicates that genes from one organism can inhibit the gene expression of an organism from another kingdom.

TABLE 4
AT #88 Amino acid sequence comparison

| Clone | | | |
|---|---|---|---|
| Positives | Score | pValue | Identities |
| A.. thaliana AC004625 | 430 (151.4 bits) | 4.40E−52 | 70/70 (100%) |
| 70/70 (100%) | | | |
| D. discoideum | 246 (86.6 bits) | 2.60E−20 | 58/98 (59%) |
| ANNEXIN VII P24639 | | | |
| 62/98 (63%) | | | |
| D. discoideum | 245 (86.2 bits) | 3.00E−20 | 57/91 (62%) |
| ANNEXIN VII X60270 | | | |
| 60/91 (65%) | | | |
| Octopus rhodopsin | 235 (82.7 bits) | 4.00E−19 | 50/85 (58%) |
| X07797 | | | |
| 54/85 (63%) | | | |

TABLE 5
AT #2483 Nucleotide sequence comparison

| Clone | Score | pValue | Identities | Positives |
|---|---|---|---|---|
| A.. thaliana AF075597 | 389 (107.5 bits) | 2.60E−38 | 101/130 (77%) | 101/130 (77%) |
| Rice mRNA for ribosomal protein L19 D21304 | 198 (54.7 bits) | 2.20E−10 | 50/64 (78%) | 50/64 (78%) |
| D. melanogaster rib, protein L19 mRNA L32181 | 185 (51.1 bits) | 3.40E−09 | 49/64 (76%) | 49/64 (76%) |
| N. tabacum L19 mRNA Z31720 | 194 (53.6 bits) | 3.50E−05 | 50.64 (78%) | 50/64 (78%) |
| Mus musculus ribosomal protein L19 M62952 | 166 (45.9 bits) | 4.40E−04 | 42/53 (79%) | 42/53 (79%) |
| Human ribosomal protein L19 S56985 | 153 (42.3 bits) | 8.30E−02 | 45/63 (71%) | 45/63 (71%) |

TABLE 6
AT #2483 Amino acid sequence comparison

| Clone | Score | pValue | Identities | Positives |
|---|---|---|---|---|
| S. pombe ribosomal protein L19 042699 | 56 (25.8 bits) | 5.50E−09 | 12/31 (38%) | 12/31 (38%) |
| Human ribosmal protein L19 P14118 | 77 (35.4 bits) | 8.20E−09 | 15/18 (83%) | 15/18 (83%) |
| M. musculus ribosomal protein L19 P22908 | 77 (35.4 bits) | 8.20E−09 | 15/18 (83%) | 15/18 (83%) |
| D. melanogaster ribosomal protein L19 | 70 (36.3 bits) | 1.50E−08 | 16/18 (88%) | 16/18 (88%) |

### Example 20

Novel requirements for production of infectious viral vector in vitro derived RNA transcripts.

This example demonstrates the production of highly infectious viral vector transcripts containing 5′ nucleotides with reference to the virus vector.

Construction of a library of subgenomic cDNA clones of TMV and BMV has been described in Dawson et al., Proc. Natl. Acad. Sci. USA 83:1832-1836 (1986) and Ahlquist et al., Proc. Natl. Acad. Sci. USA 81:7066-7070 (1984). Nucleotides were added between the transcriptional start site of the promoter for in vitro transcription, in this case T7, and the start of the cDNA of TMV in order to maximize transcription product yield and possibly obviate the need to cap virus transcripts to insure infectivity. The relevant sequence is the T7 promoter . . . TATAGˆ TATTTT (SEQ ID NO: 46) . . . where the ˆ indicates the base preceding, is the start site for transcription and the bold letter is the first base of the TMV cDNA. Three approaches were taken: 1) addition of G, GG or GGG between the start site of transcription and the TMV cDNA (. . . TATAGGTATTT, SEQ ID NO: 47, . . . and associated sequences); 2) addition of G and a random base (GN or N2) or a G and two random bases (GNN or N3) between the start site of transcription and the TMV cDNA (. . . TATAGNTATTT, SEQ ID NO: 48, . . . and associated sequences), and the addition of a GT and a single random base between the start site of transcription and the TMV cDNA (. . . TATAGTNGTATTT, SEQ ID NO: 49 . . . and associated sequences). The use of random bases was based on the hypothesis that a particular base may be best suited for an additional nucleotide attached to the cDNA, since it will be complementary to the normal nontemplated base incorporated at the 3′-end of the TMV (−) strand RNA. This allows for more ready mis-initiation and restoration of wild type sequence. The GTN would allow the mimicking of two potential sites for initiation, the added and the native sequence, and facilitate more ready mis-initiation of transcription in vivo to restore the native TMV cDNA sequence. Approaches included cloning GFP expressing TMV vector sequences into vectors containing extra G, GG or GGG bases using standard molecular biology techniques. Likewise, full length PCR of TMV expression clone 1056 was done to add N2, N3 and GTN bases between the T7 promoter and the TMV cDNA. Subsequently, these PCR products were cloned into pUC based vectors. Capped and uncapped transcripts were made in vitro and inoculated to tobacco protoplasts or Nicotiana benthamiana plants, wild type and 30 k expressing transgenics. The results are that an extra G . . . TATAGGTATTTT, SEQ ID NO: 50, . . . , or a GTC, . . . TATAGTCGTATTTT, SEQ ID NO: 51, . . . , were found to be well tolerated as additional 5′ nucleotides on the 5′ of TMV vector RNA transcripts and were quite infectious on both plant types and protoplasts as capped or non-capped transcripts. Other sequences may be screened to find other options. Clearly, infectious transcripts may be derived with extra 5′ nucleotides.

Other derivatives based on the putative mechanistic function of the GTN strategy that yielded the GTC functional vector are to use multiple GTN motifs preceding the 5′ most nt of the virus cDNA or the duplication of larger regions of the 5′-end of the TMV genome. For example: TATAˆ GTNGTNGTATT, SEQ ID NO: 52, or TATAˆ GTNGTNGTNGTNGTATT, SEQ ID NO: 53, . . . or TATAˆ GTATTTGTATTT, SEQ ID NO: 54, . . . In this manner the replication mediated repair mechanism may be potentiated by the use of multiple recognition sequences at the 5′-end of transcribed RNA. The replicated progeny may exhibit the results of reversion events that would yield the wild type virus 5′ virus sequence, but may include portions or entire sets of introduced additional base sequences. This strategy can be applied to a range of RNA viruses or RNA viral vectors of various genetic arrangements derived from wild type virus genome. This would require the use of sequences particular to that of the virus used as a vector.

### Example 21

Infectivity of uncapped transcripts.

Two TMV-based virus expression vectors were initially used in these studies pBTI 1056 which contains the T7 promoter followed directly by the virus cDNA sequence ( . . . TATAGTATT . . . ), and pBTI SBS60-29 which contains the T7 promoter (underlined) followed by an extra guanine residue then the virus cDNA sequence ( . . . TATAGGTATTT . . . ). Both expression vectors express the cycle 3 shuffled green fluorescent protein (GFPc3) in localized infection sites and systemically infected tissue of infected plants. Transcriptions of each plasmid were carried out in the absence of cap analogue (uncapped) or in the presence of 8-fold greater concentration of RNA cap analogue than rGTP (capped). Transcriptions were mixed with abrasive and inoculated on expanded older leaves of a wild type Nicotiana benthamiana (Nb) plant and a Nb plant expressing a TMV U1 30 k movement protein transgene (Nb 30K). Four days post inoculation (dpi), long wave UV light was used to judge the number of infection sites on the inoculated leaves of the plants. Systemic, noninoculated tissues, were monitored from 4 dpi on for appearance of systemic infection indicating vascular movement of the inoculated virus. Table 7 shows data from one representative experiment.

TABLE 7

| | Local infection sites | | Systemic Infection | |
|---|---|---|---|---|
| Construct | Nb | Nb 30 K | Nb | Nb 30 K |
| pBTI1056 | | | | |
| Capped | 5 | 6 | yes | yes |
| Uncapped | 0 | 5 | no | yes |
| PBTI SBS60-29 | | | | |
| Capped | 6 | 6 | yes | yes |
| Uncapped | 1 | 5 | yes | yes |

Nicotiana tabacum protoplasts were infected with either capped or uncapped transcriptions (as described above) of pBTI SBS60 which contains the T7 promoter followed directly by the virus cDNA sequence (TATAGTATT . . . ). This expression vector also expresses the GFPc3 gene in infected cells and tissues. Nicotiana tabacum protoplasts were transfected with 1 mcl of each transcriptions. Approximately 36 hours post infection transfected protoplasts were viewed under UV illumination and cells showing GFPc3 expression. Approximately 80% cells transfected with the capped PBTI SBS60 transcripts showed GFP expression while 5% of cells transfected with uncapped transcripts showed GFP expression. These experiments were repeated with higher amounts of uncapped inoculum. In this case a higher proportion of cells, >30% were found to be infected at this time with uncapped transcripts, where >90% of cells infected with greater amounts of capped transcripts were scored infected.

These results indicate that, contrary to the practiced art in scientific literature and in issued patents (Ahlquist et al., U.S. Pat. No. 5,466,788), uncapped transcripts for virus expression vectors are infective on both plants and in plant cells, however with much lower specific infectivity. Therefore, capping is not a prerequisite for establishing an infection of a virus expression vector in plants; capping just increases the efficiency of infection. This reduced efficiency can be overcome, to some extent, by providing excess in vitro transcription product in an infection reaction for plants or plant cells.

The expression of the 30K movement protein of TMV in transgenic plants also has the unexpected effect of equalizing the relative specific infectivity of uncapped verses capped transcripts. The mechanism behind this effect is not fully understood, but could arise from the RNA binding activity of the movement protein stabilizing the uncapped transcript in infected cells from prereplication cytosolic degradation.

Extra guanine residues located between the T7 promoter and the first base of a virus cDNA lead to increased amount of RNA transcript as predicted by previous work with phage polymerases. These polymerases tend to initiate more efficiently at . . . TATAGG or . . . TATAGGG than . . . TATAG. This has an indirect effect on the relative infectivity of uncapped transcripts in that greater amounts are synthesized per reaction resulting in enhanced infectivity.

Data concerning cap dependent transcription of pBTI1056 GTN#28.

TMV-based virus expression vector pBTI 1056 GTN#28 which contains the T7 promoter (underlined) followed GTC bases (bold) then the virus cDNA sequence (. . . TATAGTCGTATT, SEQ ID NO: 55, . . . ). This expression vector expresses the cycle 3 shuffled green fluorescent protein (GFPc3) in localized infection sites and systemically infected tissue of infected plants. This vector was transcribed in vitro in the presence (capped) and absence (uncapped) of cap analogue. Transcriptions were mixed with abrasive and inoculated on expanded older leaves of a wild type Nicotiana benthamiana (Nb) plant and a Nb plant expressing a TMV Ul 30k movement protein transgene (Nb 30K). Four days post inoculation (dpi) long wave UV light was used to judge the number of infection sites on the inoculated leaves of the plants. Systemic, non-inoculated tissues, were monitored from 4 dpi on for appearance of systemic infection indicating vascular movement of the inoculated virus. Table 8 shows data from two representative experiments at 11 dpi.

TABLE 8

| | Local infection sites | | Systemic Infection | |
|---|---|---|---|---|
| Construct | Nb | Nb 30 K | Nb | Nb 30 K |
| Experiment 1 | | | | |
| pBTI1056 GTN#28 | | | | |
| Capped | 18 | 25 | yes | yes |
| Uncapped | 2 | 4 | yes | yes |
| Experiment 2 | | | | |
| pBTI1056 GTN#28 | | | | |
| Capped | 8 | 12 | yes | yes |
| Uncapped | 3 | 7 | yes | yes |

These data further support the claims concerning the utility of uncapped transcripts to initiate infections by plant virus expression vectors and further demonstrates that the introduction of extra, non-viral nucleotides at the 5′-end of in vitro transcripts does not preclude infectivity of uncapped transcripts.

What is claimed is:

1. A method of isolating human cDNAs, said method comprising the steps of:
(a) obtaining a cDNA library from a human organism, (b) constructing recombinant viral nucleic acids comprising a nucleic acid insert derived from said library,
(c) infecting a host plant with said recombinant viral nucleic acids, and expressing transiently said nucleic acid in an antisense orientation in said host plant,
(d) growing said infected host plant,
(e) detecting one or more changes in said host plant,
(f) identifying said recombinant viral nucleic acid that results in changes in said host plant,
(g) sequencing and labeling said nucleic acid insert in said recombinant viral nucleic acid of (f),
(h) probing filters or slides containing full-length human cDNAs with said labeled nucleic acid insert, and
(i) isolating said full-length human cDNA that hybridizes to said labeled nucleic acid insert.

2. The method according to claim 1, wherein said nucleic acid insert encodes a protein that regulates growth of cells or organisms in human.

3. The method according to claim 2, wherein said protein is a L19 ribosomal protein, a GTP binding protein, or a S18 ribosomal protein.

4. The method according to claim 1, wherein said nucleic acid insert encodes a protein that regulates a development fate in human.

5. The method according to claim 4, wherein said protein belongs to a rhodopsin family.

6. The method according to claim 1, wherein said recombinant viral nucleic acids are derived from a tobamovirus.

7. The method according to claim 6, wherein said tobamovirus is a tobacco mosaic virus.
